(19) United States
(12) Patent Application Publication
(10) Pub. No.: US 2007/0275984 A1
(43) Pub. Date: Nov. 29, 2007

(54) Novel Thieno-Pyridine and Thieno-Pyrimidine Derivatives and Their Use as Positive Allosteric Modulators of Mglur2-Receptors

(75) Inventors: Hassan Julien Imogai, Geneva (CH); Vincent Mutel, Geneva (CH); Guillaume Albert Jacques Duvey, Geneva (CH); Jose Maria Cid-Nunez, Toledo (ES); Emmanuel Christian Le Poul, Geneva (CH); Robert Johannes Lutjens, Geneva (CH)

Correspondence Address: GARDNER GROFF GREENWALD & VILLANUEVA. PC, 2018 POWERS FERRY ROAD, SUITE 800, ATLANTA, GA, 30339, US

(73) Assignees: JANSSEN PHARMACEUTICA N.V., Beerse (BE); ADDEX PHARMACEUTICALS S.A., Plan-les-Ouates (Geneva) (CH)

(21) Appl. No.: 11/575,432

(22) PCT Filed: Sep. 16, 2005
(86) PCT No.: PCT/EP05/54635

§ 371 Date: Mar. 16, 2007

(30) Foreign Application Priority Data
Sep. 17, 2004 (GB) 0420719.7

(51) Int. Cl. A61K 31/519; A61K 31/4365; A61P 25/00; A61P 25/06; A61P 25/08; A61P 25/18; A61P 25/30; C07D 491/02; C07D 513/04

(52) U.S. Cl. 514/260.1; 514/301; 544/278; 546/114

(57) ABSTRACT

The present invention relates to novel compounds, in particular novel thieno-pyridine and thieno-pyrimidine derivatives according to Formula (I), wherein all radicals are defined in the application. The compounds according to the invention are positive allosteric modulators of metabotropic receptors—subtype 2 (“mGluR2”) which are useful for the treatment or prevention of neurological and psychiatric disorders associated with glutamate dysfunction and diseases in which the mGluR2 subtype of metabotropic receptors is involved. In particular, such diseases are central nervous system disorders selected from the group of anxiety, schizophrenia, migraine, depression, and epilepsy. The invention is also directed to pharmaceutical compositions and processes to prepare such compounds and compositions, as well as to the use of such compounds for the prevention and treatment of such diseases in which mGluR2 is involved.

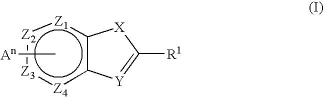

### SUMMARY OF THE INVENTION

The present invention relates to novel compounds, in particular novel thieno-pyridine and thieno-pyrimidine derivatives that are positive allosteric modulators of metabotropic receptors—subtype 2 (“mGluR2”) which are useful for the treatment or prevention of neurological and psychiatric disorders associated with glutamate dysfunction and diseases in which the mGluR2 subtype of metabotropic receptors is involved. The invention is also directed to the pharmaceutical compositions, the processes to prepare such compounds and compositions and the use of such compounds for the prevention and treatment of such diseases in which mGluR2 is involved.

### BACKGROUND OF THE INVENTION

Glutamate is the major amino-acid transmitter in the mammalian central nervous system (CNS). Glutamate plays a major role in numerous physiological functions, such as learning and memory but also sensory perception, development of synaptic plasticity, motor control, respiration, and regulation of cardiovascular function. Furthermore, glutamate is at the centre of several different neurological and psychiatric diseases, where there is an imbalance in glutamatergic neurotransmission.

Glutamate mediates synaptic neurotransmission through the activation of ionotropic glutamate receptors channels (iGluRs), the NMDA, AMPA and kainate receptors which are responsible for fast excitatory transmission (Nakanishi et al., (1998) Brain Res Brain Res Rev., 26:230-235).

In addition, glutamate activates metabotropic glutamate receptors (mGluRs) which have a more modulatory role that contributes to the fine-tuning of synaptic efficacy.

The mGluRs are seven-transmembrane G protein-coupled receptors (GPCRs) belonging to family 3 of GPCRs along with the calcium-sensing, GABAb, and pheromone receptors.

Glutamate activates the mGluRs through binding to the large extracellular amino-terminal domain of the receptor, herein called the orthosteric binding site. This binding induces a conformational change in the receptor which results in the activation of the G-protein and intracellular signalling pathways.

The mGluR family is composed of eight members. They are classified into three groups (group I comprising mGluR1 and mGluR5; group II comprising mGluR2 and mGluR3; group III comprising mGluR4, mGluR6, mGluR7, and mGluR8) according to sequence homology, pharmacological profile, and nature of intracellular signalling cascades activated (Schoepp et al. (1999) Neuropharmacology, 38:1431-76).

Among mGluR members, the mGluR2 subtype is negatively coupled to adenylate cyclase via activation of Gαi-protein, and its activation leads to inhibition of glutamate release in the synapse (Cartmell & Schoepp (2000) J Neurochem 75:889-907). In the CNS, mGluR2 receptors are abundant mainly throughout cortex, thalamic regions, accessory olfactory bulb, hippocampus, amygdala, caudate-putamen and nucleus accumbens (Ohishi et al. (1998) Neurosci Res 30:65-82).

Activating mGluR2 was shown in clinical trials to be efficacious to treat anxiety disorders (Levine et al. (2002) Neuropharmacology 43:294; Holden (2003) Science 300:1866-68; Grillon et al. (2003) Psychopharmacology 168:446-54; Kellner et al. (2005) Psychopharmacology 179: 310-15). In addition, activating mGluR2 in various animal models was shown to be efficacious, thus representing a potential novel therapeutic approach for the treatment of schizophrenia (reviewed in Schoepp & Marek (2002) Curr Drug Targets. 1:215-25), epilepsy (reviewed in Moldrich et al. (2003) Eur J Pharmacol. 476:3-16), migraine (Johnson et al. (2002) Neuropharmacology 43:291), addiction/drug dependence (Helton et al. (1997) J Pharmacol Exp Ther 284: 651-660), Parkinson's disease (Bradley et al (2000) J Neurosci. 20(9):3085-94), pain (Simmons et al. (2002) Pharmacol Biochem Behav 73:419-27), sleep disorders (Feinberg et al. (2002) Pharmacol Biochem Behav 73:467-74) and Huntington's disease (Schiefer et al. (2004) Brain Res 1019:246-54).

To date, most of the available pharmacological tools targeting mGluRs are orthosteric ligands which activate several members of the family as they are structural analogs of glutamate (Schoepp et al. (1999) Neuropharmacology, 38:1431-76).

A new avenue for developing selective compounds acting at mGluRs is to identify molecules that act through allosteric mechanisms, modulating the receptor by binding to a site different from the highly conserved orthosteric binding site.

Positive allosteric modulators of mGluRs have emerged recently as novel pharmacological entities offering this attractive alternative. This type of molecule has been discovered for several mGluRs (reviewed in Mutel (2002) Expert Opin. Ther. Patents 12:1-8). In particular molecules have been described as mGluR2 positive allosteric modulators (Johnson M P et al. (2003) J Med Chem. 46:3189-92; Pinkerton et al. (2004) J Med Chem. 47:4595-9).

WO2004092135 (NPS & Astra Zeneca), WO04018386 (Merck) and WO0156990 (Eli Lilly) describe respectively phenyl sulfonamide, acetophenone and pyridylmethyl sulfonamide derivatives as mGluR2 positive allosteric modulators. However, none of the specifically disclosed compounds are structurally related to the compounds of the invention.

It was demonstrated that such molecules do not activate the receptor by themselves (Johnson M P et al. (2003) J Med. Chem. 46:3189-92; Schaffhauser et al. (2003) Mol Pharmacol. 64:798-810). Rather, they enable the receptor to produce a maximal response to a concentration of glutamate which by itself induces a minimal response. Mutational analysis have demonstrated unequivocally that the binding of mGluR2 positive allosteric modulators does not occur at the orthosteric site, but instead at an allosteric site situated within the seven transmembrane region of the receptor (Schaffhauser et al. (2003) Mol Pharmacol. 64:798-810).

Animal data are suggesting that positive allosteric modulators of mGluR2 have the same effects in anxiety and psychosis models as those obtained with orthosteric agonists. Allosteric modulators of mGluR2 were shown to be active in fear-potentiated startle (Johnson et al. (2003) J Med Chem. 46:3189-92; Johnson et al. (2005) Psychopharmacology 179:271-83), and in stress-induced hyperthermia (Johnson et al. (2005) Psychopharmacology 179:271-83) models of anxiety. Furthermore, such compounds were shown to be active in reversal of ketamine- (Govek et al. (2005) Bioorg Med Chem Lett 15(18):4068-72) or amphetamine- (Galici et al. (2005) J Pharm Exp Ther Fast Forward, 2005 Aug. 25, Epub ahead of print) induced hyperlocomotion, and in reversal of amphetamine-induced disruption of prepulse inhibition of the acoustic startle effect (Galici et al. J Pharm Exp Ther Fast Forward, 2005 Aug. 25, Epub ahead of print) models of schizophrenia.

Positive allosteric modulators enable potentiation of the glutamate response, but they have also been shown to potentiate the response to orthosteric mGluR2 agonists such as LY379268 (Johnson et al. (2004) Biochem Soc Trans 32:881-87) or DCG-IV (Poisik et al. (2005) Neuropharmacology 49:57-69). These data provide evidence for yet another novel therapeutic approach to treat above mentioned neurological diseases involving mGluR2, which would use a combination of a positive allosteric modulator of mGluR2 together with an orthosteric agonist of mGluR2.

### DETAILED DESCRIPTION OF THE INVENTION

The invention relates to compounds having metabotropic glutamate receptor 2 modulator activity. In its most general compound aspect the present invention provides a compound according to Formula (I), 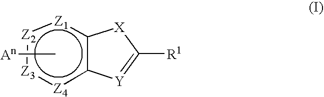
a pharmaceutically acceptable acid or base addition salt thereof, a stereochemically isomeric form thereof and an N-oxide form thereof, wherein:
Y is selected from —N— and —C(R²)—;
X is selected from —S—, —S(O)—, —S(O)₂—, —O— and —N(R³)—;
R¹, R² and R³ are each independently selected from the group of hydrogen, halo, —CN, —OH, —NO2, —CF₃, —NH₂, —SH, —C(═NR⁴)NR⁵R⁶, —C(═O)R⁴, —C(═NR⁴)R⁵, —C(═O)OR⁴, —C(═O)NR⁴R⁵, —SR⁴, —S(O)R⁴, —S(O)₂R⁴, —NR⁴R⁵, —NR⁴C(═O)R⁵, —NR⁴C(═NR⁵)R⁶, —NR⁴C(═NR⁵)NR⁶R⁷, —NR⁴C(═O)OR⁵, —NR⁴C(═O)NR⁵R⁶, —NR⁴S(O)₂R⁵, —S(O)₂NR⁴R⁵, —C(═S)NR⁴R⁵, —OC(═O)R⁴, —OC(═O)NR⁴R⁵, —OR⁴, an optionally substituted radical selected from the group of —(C₁-C₆)alkyl, —(C₁-C₆)alkylhalo, —(C₂-C₆)alkynyl, —(C₂-C₆)alkenyl, —(C₃-C₇)cycloalkyl, —(C₃-C₈)cycloalkenyl, —(C₁-C₆)alkylcyano, —(C₁-C₆)alkylaryl, —(C₁-C₆)alkylheteroaryl, aryl and heteroaryl, and a radical described as —V₁-T₁-M₁;
Z₁, Z₂, Z₃ and Z₄ are each independently selected from a covalent bond, C, S, N and O, with the provision that a 5 or 6 membered heteroaryl or aryl ring is formed, which may optionally be substituted by 1 to 4 radicals Aⁿ;
Aⁿ radicals are each independently selected from the group of hydrogen, halo, —CN, —OH, —NO₂, —CF₃, —SH, —NH₂, an optionally substituted radical selected from the group of —(C₁-C₆)alkyl, —(C₁-C₆)alkylhalo, —(C₂-C₆)alkynyl, —(C₂-C₆)alkenyl, —(C₃-C₇)cycloalkyl, —(C₁-C₆)alkylcyano, —O—(C₁-C₆)alkyl, —O—(C₁-C₆)alkylhalo, —O—(C₁-C₆)alkylcyano, —O—(C₃-C₆)alkynyl, —O—(C₃-C₇)cycloalkyl, —O—(C₂-C₆)alkenyl, —O—(C₂-C₆)alkyl-OR⁸, —O—(C₁-C₆)alkyl-heteroaryl, —O—(C₀-C₆)alkylaryl, —(C₀-C₆)alkyl-OR⁸, —(C₃-C₇)cycloalkyl-(C₁-C₆)alkyl, —O—(C₃-C₇)cycloalkyl-(C₁-C₆)alkyl, —O-heteroaryl, heteroaryl, —(C₁-C₆)alkyl-heteroaryl, aryl, —O-aryl, —(C₁-C₆)alkylaryl, —(C₁-C₆)alkylhalo-OR⁸, —(C₃-C₆)alkynyl-OR⁸, —(C₃-C₆)alkenyl-OR⁸, —(C₀-C₆)alkyl-SR⁸, —O—(C₂-C₆)alkyl-SR⁸, —(C₁-C₆)alkyl-S(═O)—R⁸, —O—(C₁-C₆)alkyl-S(═O)—R⁸, —(C₀-C₆)alkyl-S(═O)₂—R⁸, —O—(C₁-C₆)alkyl-S(═O)₂—R⁸, —(C₀-C₆)alkyl-NR⁸R⁹, —O—(C₂-C₆)alkyl-NR⁸R⁹, —(C₀-C₆)alkyl-S(═O)₂NR⁸R⁹, —(C₀-C₆)alkyl-NR⁸—S(═O)₂R⁹, —O—(C₁-C₆)alkyl-S(═O)₂NR⁸R⁹, —O—(C₁-C₆)alkyl-NR⁸—S(═O)₂R⁹, —(C₀-C₆)alkyl-C(═O)—NR⁸R⁹, —(C₀-C₆)alkyl-NR⁸C(═O)—R⁹, —O—(C₁-C₆)alkyl-C(═O)—NR⁸R⁹, —O—(C₁-C₆)alkyl-NR⁸C(═O)—R⁹, —(C₀-C₆)alkyl-OC(═O)—R⁸, —(C₀-C₆)alkyl-C(═O)—OR⁸, —O—(C₁-C₆)alkyl-OC(═O)—R⁸—O—(C₁-C₆)alkyl-C(═O)—OR⁸, —(C₀-C₆)alkyl-C(═O)—R⁸, —O—(C₁-C₆)alkyl-C(═O)—R⁸, —(C₀-C₆)alkyl-NR⁸—C(═O)—OR⁹, —(C₀-C₆)alkyl-O—C(═O)—NR⁸R⁹, —(C₀-C₆)alkyl-NR⁸—C(═NR⁹)—NR¹⁰R¹¹, —(C₀-C₆)alkyl-NR⁸—C(═O)—NR⁹R¹⁰, —(C₀-C₆)alkyl-NR⁸—C(═S)—NR⁹R¹⁰, and a —V₂-T₂-M₂ radical;
n is an integer ranging from 1 to 4;
T₁, V₁, T₂ and V₂ are each independently selected from the group of a covalent bond, —O—, —C(═O)—, —C(═O)O—, —C(═O)NR¹²—, —S—, —S(O)—, —S(O)₂—, —S(O)₂NR¹²—, —NR¹²—, —NR¹²C(═O)—, —NR¹²C(═O)NR¹³—, —NR¹²S(O)₂—, —NR¹²C(═S)NR¹³—, —OC(═O), —OC(═O)NR¹², —NR¹²C(═O)O—, and an optionally substituted radical selected from the group of —(C₁-C₆)alkyl-, —(C₂-C₆)alkynyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₁-C₆)alkyl-, —(C₀-C₆)alkyl-O—(C₂-C₆)alkynyl-, —(C₀-C₆)alkyl-O—(C₂-C₆)alkenyl-, —(C₀-C₆)alkyl-O—(C₃-C₇)cycloalkyl-, —(C₀-C₆)alkyl-O—(C₄-C₁₀)alkylcycloalkyl-, —(C₀-C₆)alkyl-C(═O)—(C₁-C₆)alkyl-, —(C₀-C₆)alkyl-C(═O)—(C₂-C₆)alkynyl-, —(C₀-C₆)alkyl-C(═O)—(C₂-C₆)alkenyl-, —(C₀-C₆)alkyl-C(═O)—(C₄-C₁₀)alkylcycloalkyl-, —(C₀-C₆)alkyl-C(═O)—(C₃-C₇)cycloalkyl-, —(C₀-C₆)alkyl-C(═O)O—(C₁-C₆)alkyl-, —(C₀-C₆)alkyl-C(═O)O—(C₂-C₆)alkynyl-, —(C₀-C₆)alkyl-C(═O)O—(C₂-C₆)alkenyl-, —(C₀-C₆)alkyl-C(═O)O—(C₃-C₇)cycloalkyl-, —(C₀-C₆)alkyl-C(═O)O—(C₄-C₁₀)alkylcycloalkyl-, —(C₀-C₆)alkyl-C(═O)NR¹²—(C₁-C₆)alkyl-, —(C₀-C₆)alkyl-C(═O)NR¹²—(C₂-C₆)alkynyl-, —(C₀-C₆)alkyl-C(═O)NR¹²—(C₂-C₆)alkenyl-, —(C₀-C₆)alkyl-C(═O)NR¹²—(C₃-C₇)cycloalkyl-, —(C₀-C₆)alkyl-C(═O)NR¹²—(C₄-C₁₀)alkylcycloalkyl-, —(C₀-C₆)alkyl-S—(C₁-C₆)alkyl-, —(C₀-C₆)alkyl-S—(C₂-C₆)alkynyl-, —(C₀-C₆)alkyl-S—(C₂-C₆)alkenyl-, —(C₀-C₆)alkyl-S—(C₃-C₇)cycloalkyl-, —(C₀-C₆)alkyl-S—(C₄-C₁₀)alkylcycloalkyl-, —(C₀-C₆)alkyl-S(O)-(C₁-C₆)alkyl-, —(C₀-C₆)alkyl-O—(C₂-C₆)alkynyl-, —(C₀-C₆)alkyl-S(O)—(C₂-C₆)alkenyl-, —(C₀-C₆)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₁-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-S(O)₂NR¹²—(C₁-C₆)alkyl-, —(C₀-C₆)alkyl-S(O)₂NR¹²—(C₂-C₆)alkynyl-, —(C₀-C₆)alkyl-S(O)₂NR¹²—(C₂-C₆)alkenyl-, —(C₀-C₆)alkyl-S(O)₂NR¹²—(C₃-C₇)cycloalkyl-, —(C₀-C₆)alkyl-S(O)₂NR¹²—(C₄-C₁₀)alkylcycloalkyl-, —(C₀-C₆)alkyl-NR¹²—(C₁-C₆)alkyl-, —(C₀-C₆)alkyl-NR¹²—(C₂-C₆)alkynyl-, —(C₀-C₆)alkyl-NR¹²—(C₂-C₆)alkenyl-, —(C₀-C₆)alkyl-NR¹²—(C₃-C₇)cycloalkyl-, —(C₀-C₆)alkyl-NR¹²—(C₄-C₁₀)alkylcycloalkyl-, —(C₀-C₆)alkyl-NR¹²C(═O)—(C₁-C₆)alkyl-, —(C₀-C₆)alkyl-NR¹²C(═O)—(C₂-C₆)alkynyl-, —(C₀-C₆)alkyl-NR¹²C(═O)—(C₂-C₆)alkenyl-, —(C₀-C₆)alkyl-NR¹²C(═O)—(C₃-C₇)cycloalkyl-, —(C₀-C₆)alkyl-NR¹²C(═O)—(C₄-C₁₀)alkylcycloalkyl-, —(C₀-C₆)alkyl-NR¹²C(═O)NR¹³—(C₁-C₆)alkyl-, —(C₀-C₆)alkyl-NR¹²C(═O)NR¹³—(C₂-C₆)alkynyl-, —(C₀-C₆)alkyl-NR¹²C(═O)NR¹³—(C₂-C₆)alkenyl-, —(C₀-C₆)alkyl-NR¹²C(═O)NR¹³—(C₃-C₇)cycloalkyl-, —(C₀-C₆)alkyl-NR¹²C(═O)NR¹³—(C₄-C₁₀)alkylcycloalkyl-, —(C₀-C₆)alkyl-NR¹²S(O)₂—(C₁-C₆)alkyl-, —(C₀-C₆)alkyl-NR¹²S(O)₂—(C₂-C₆)alkynyl-, —(C₀-C₆)alkyl-NR¹²S(O)₂—(C₂-C₆)alkenyl-, —(C₀-C₆)alkyl-NR¹²S(O)₂—(C₃-C₇)cycloalkyl-, —(C₀-C₆)alkyl-NR¹²S(O)₂—(C₄-C₁₀)alkylcycloalkyl-, —(C₀-C₆)alkyl-NR¹²C(═S)NR¹³—(C₁-C₆)alkyl-, —(C₀-C₆)alkyl-NR¹²C(═S)NR¹³—(C₂-C₆)alkynyl-, —(C₀-C₆)alkyl-NR¹²C(═S)NR¹³—(C₂-C₆)alkenyl-, —(C₀-C₆)alkyl-NR¹²C(═S)NR¹³—(C₃-C₇)cycloalkyl-, —(C₀-C₆)alkyl-NR¹²C(═S)NR¹³—(C₄-C₁₀)alkylcycloalkyl-, —(C₀-C₆)alkyl-OC(═O)—(C₁-C₆)alkyl-, —(C₀-C₆)alkyl-OC(═O)—(C₂-C₆)alkynyl-, —(C₀-C₆)alkyl-OC(═O)—(C₂-C₆)alkenyl-, —(C₀-C₆)alkyl-OC(═O)—(C₄-C₁₀)alkylcycloalkyl-, —(C₀-C₆)alkyl-OC(═O)—(C₃-C₇)cycloalkyl-, —(C₀-C₆)alkyl-OC(═O)NR¹²—(C₁-C₆)alkyl-, —(C₀-C₆)alkyl-OC(═O)NR¹²—(C₂-C₆)alkynyl-, —(C₀-C₆)alkyl-OC(═O)NR¹²—(C₂-C₆)alkenyl-, —(C₀-C₆)alkyl-OC(═O)NR¹²—(C₄-C₁₀)alkylcycloalkyl-, —(C₀-C₆)alkyl-OC(═O)NR¹²—(C₃-C₇)cycloalkyl-, —(C₀-C₆)alkyl-NR¹²C(═O)O—(C₁-C₆)alkyl-, —(C₀-C₆)alkyl-NR¹²C(═O)O—(C₂-C₆)alkynyl-, —(C₀-C₆)alkyl-NR¹²C(═O)O—(C₂-C₆)alkenyl-, —(C₀-C₆)alkyl-NR¹²C(═O)O—(C₃-C₇)cycloalkyl-, —(C₀-C₆)alkyl-NR¹²C(═O)O—(C₄-C₁₀)alkylcycloalkyl-, —(C₀-C₆)alkyl-NR¹²C(═NR¹³)NR¹⁴—(C₁-C₆)alkyl-, —(C₀-C₆)alkyl-NR¹²C(═NR¹³)NR¹⁴—(C₂-C₆)alkynyl-, —(C₀-C₆)alkyl-NR¹²C(═NR¹³)NR¹⁴—(C₂-C₆)alkenyl-, —(C₀-C₆)alkyl-NR¹²C(═NR¹³)NR¹⁴—(C₃-C₇)cycloalkyl-, —(C₀-C₆)alkyl-NR¹²C(═NR¹³)NR¹⁴—(C₄-C₁₀)alkylcycloalkyl-, —(C₀-C₆)alkyl-NR¹²C(═NR¹³)—(C₁-C₆)alkyl-, —(C₀-C₆)alkyl-NR¹²C(═NR¹³)—(C₂-C₆)alkynyl-, —(C₀-C₆)alkyl-NR¹²C(═NR¹³)—(C₂-C₆)alkenyl-, —(C₀-C₆)alkyl-NR¹²C(═NR¹³)—(C₃-C₇)cycloalkyl-, —(C₀-C₆)alkyl-NR¹²C(═NR¹³)—(C₄-C₁₀)alkylcycloalkyl-, —(C₀-C₆)alkyl-C(═NR¹²)NR¹³—(C₀-C₆)alkyl-, —(C₀-C₆)alkyl-C(═NR¹²)NR¹³—(C₂-C₆)alkynyl-, —(C₀-C₆)alkyl-C(═NR¹²)NR¹³—(C₂-C₆)alkenyl-, —(C₀-C₆)alkyl-C(═NR¹²)NR¹³—(C₃-C₇)cycloalkyl- and —(C₀-C₆)alkyl-C(═NR¹²)NR¹³—(C₄-C₁₀)alkylcycloalkyl-;
M₁ and M₂ are each independently selected from the group of hydrogen, —CN, —OH, —NO₂, —CF₃, —NH₂, —SH, —C(═O)R¹⁵, —C(═NR¹⁵)R⁶, —C(═O)OR¹⁵, —C(═O)NR¹⁵R¹⁶, —SR¹⁵, —S(O)R¹⁵, —S(O)₂R¹⁵, —NR¹⁵R¹⁶, —NR¹⁵C(═O)R⁶, NR¹⁵C(═NR¹⁶)R¹⁷—NR¹⁵C(═NR¹⁶)NR¹⁷R¹⁸, —NR¹⁵C(═O)OR¹⁶, —NR¹⁵C(═O)NR¹⁶R¹⁷, —NR¹⁵S(O)₂R¹⁶, —C(═S)NR¹⁵R¹⁶, —OC(═O)R¹⁵, —OC(═O)NR¹⁵R¹⁶, —OR¹⁵, —S(O)₂NR¹⁵R¹⁶, an optionally substituted radical selected from the group of —(C₁-C₆)alkyl, —(C₂-C₆)alkynyl, —(C₂-C₆)alkenyl, —(C₃-C₇)cycloalkyl and —(C₃-C₈)cycloalkenyl, and an optionally substituted 3 to 10 membered ring selected from the group of aryl, heteroaryl, heterocyclic and cycloalkyl rings;
R⁴, R⁵, R⁶, R⁷, R⁸, R⁹, R¹⁰, R¹¹, R¹², R¹³, R¹⁴, R¹⁵, R¹⁶, R¹⁷ and R¹⁸ are each independently hydrogen or an optionally substituted radical selected from the group of —(C₁-C₆)alkylhalo, —(C₁-C₆)alkyl, —(C₁-C₆)alkylcyano, —(C₂-C₆)alkynyl, —(C₂-C₆)alkenyl, —(C₃-C₇)cycloalkyl, —(C₄-C₁₀)alkylcycloalkyl, heteroaryl, —(C₁-C₆)alkylheteroaryl, aryl, —(C₁-C₆)alkylaryl, —(C₂-C₆)alkynyl-(C₃-C₇)cycloalkyl, —(C₂-C₆)alkynyl-heteroaryl, —(C₂-C₆)alkynyl-aryl, —(C₂-C₆)alkenyl-(C₃-C₇)cycloalkyl, —(C₂-C₆)alkenyl-heteroaryl and —(C₂-C₆)alkenyl-aryl;
R⁴, R⁵, R⁶ and R⁷ may be taken together to form an optionally substituted 3 to 10 membered non-aromatic heterocyclic ring or an optionally substituted 5 to 10 membered aromatic heterocyclic ring;
R⁸, R⁹, R¹⁰ and R¹¹ may be taken together to form an optionally substituted 3 to 10 membered non-aromatic heterocyclic ring or an optionally substituted 5 to 10 membered aromatic heterocyclic ring;
R¹², R¹³ and R¹⁴ may be taken together to form an optionally substituted 3 to 10 membered non-aromatic heterocyclic ring or an optionally substituted 5 to 10 membered aromatic heterocyclic ring; and
R¹⁵, R¹⁶, R¹⁷ and R¹⁸ may be taken together to form an optionally substituted 3 to 10 membered non-aromatic heterocyclic ring or an optionally substituted 5 to 10 membered aromatic heterocyclic ring.

In one preferred aspect of Formula (I), the invention provides a compound according to Formula (II), 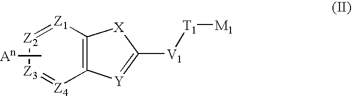
a pharmaceutically acceptable acid or base addition salt thereof, a stereochemically isomeric form thereof and an N-oxide form thereof, wherein:
Z¹, Z², Z³ and Z⁴ are each independently selected from C and N, with the provision that a 5 or 6 membered heteroaryl or aryl ring is formed, which may optionally be substituted by 1 to 4 radicals Aⁿ; and the radical 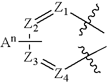
is selected from the group of radicals (a-1), (a-2), (a-3), (a-4), (a-5), (a-6) and (a-7); (a-1) (a-2) (a-3) (a-4) (a-5) (a-6) (a-7) 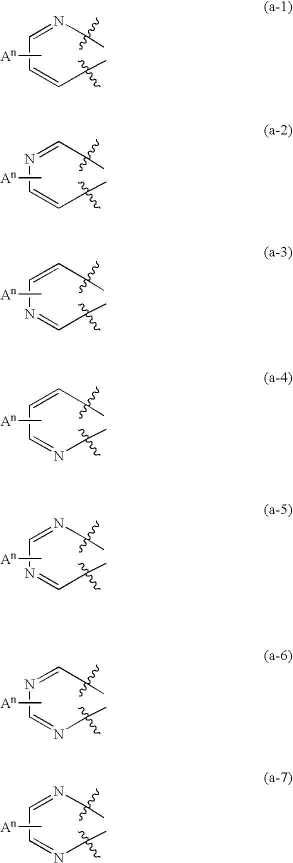
the radical 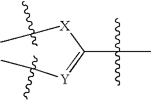
is selected from the group of radicals (b-1), (b-2), (b-3), (b-4), (b-5) and (b-6.

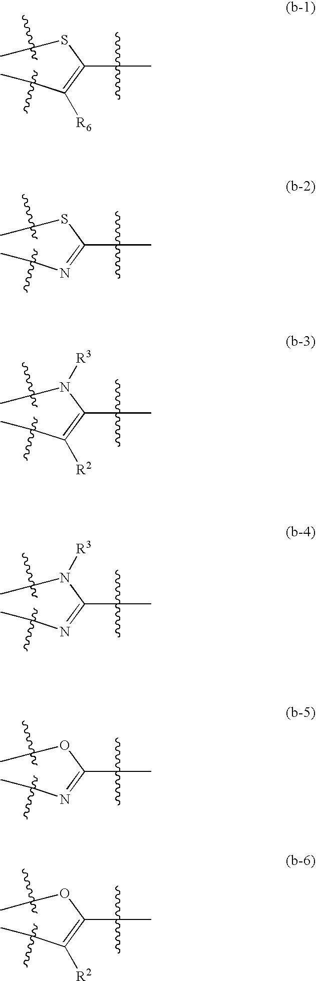

All other radicals are defined as in Formula (I).

Preferred structures according to Formula (II) are indicated in Figure A below.

[image]

In a first preferred aspect of Formula (II), the invention provides a compound according to Formula (II-a), 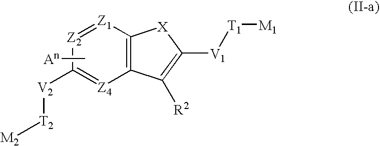
a pharmaceutically acceptable acid or base addition salt thereof, a stereochemically isomeric form thereof and an N-oxide form thereof, wherein:
R² is selected from the group of hydrogen, halo, —CN, —OH, —NO2, —CF₃, —NH₂, —SH, —C(═NR⁴)NR⁵R⁶, —C(═O)R⁴, —C(═NR⁴)R⁵, —C(═O)OR⁴, —C(═O)NR⁴R⁵, —SR4, —S(O)R⁴, S(O)₂R⁴, —NR⁴R⁵, —NR⁴C(═O)R⁵, —NR⁴C(═NR⁵)R⁶, —NR⁴C(═NR⁵)NR⁶R⁷—NR⁴C(═O)OR⁵, —NR⁴C(═O)NR⁵R⁶, —NR⁴S(O)₂R⁵, —S(O)₂NR⁴R⁵, —C(═S)NR⁴R⁵, —OC(═O)R⁴, —OC(═O)NR⁴R⁵, —OR⁴, and an optionally substituted radical selected from the group of —(C₁-C₆)alkyl, —(C₁-C₆)alkylhalo, —(C₂-C₆)alkynyl, —(C₂-C₆)alkenyl, —(C₃-C₇)cycloalkyl, —(C₃-C₈)cycloalkenyl, —(C₁-C₆)alkylcyano, —(C₁-C₆)alkylaryl, —(C₁-C₆)alkylheteroaryl, aryl and heteroaryl;
Aⁿ radicals are each independently selected from the group of hydrogen, halo, —CN, —OH, —NO₂, —CF₃, —SH, —NH₂ and an optionally substituted radical selected from the group of —(C₁-C₆)alkyl, —(C₁-C₆)alkylhalo, —(C₂-C₆)alkynyl, —(C₂-C₆)alkenyl, —(C₃-C₇)cycloalkyl, —(C₁-C₆)alkylcyano, —O—(C₁-C₆)alkyl, —O—(C₁-C₆)alkylhalo, —O—(C₁-C₆)alkylcyano, —O—(C₃-C₆)alkynyl, —O—(C₃-C₇)cycloalkyl, —O—(C₂-C₆)alkenyl, —O—(C₂-C₆)alkyl-OR⁸, —O—(C₁-C₆)alkyl-heteroaryl, —O—(C₀-C₆)alkylaryl, —(C₀-C₆)alkyl-OR⁸, —(C₃-C₇)cycloalkyl-(C₁-C₆)alkyl, —O—(C₃-C₇)cycloalkyl-(C₁-C₆)alkyl, —O-heteroaryl, heteroaryl, —(C₁-C₆)alkyl-heteroaryl, aryl, —O-aryl, —(C₁-C₆)alkylaryl, —(C₁-C₆)alkylhalo-OR⁸, —(C₃-C₆)alkynyl-OR⁸, —(C₃-C₆)alkenyl-OR⁸, —(C₀-C₆)alkyl-SR⁸, —O—(C₂-C₆)alkyl-SR⁸, —(C₁-C₆)alkyl-S(═O)—R⁸, —O—(C₁-C₆)alkyl-S(═O)—R⁸, —(C₀-C₆)alkyl-S(═O)₂—R⁸, —O—(C₁-C₆)alkyl-S(═O)₂—R⁸, —(C₀-C₆)alkyl-NR⁸R⁹, —O—(C₂-C₆)alkyl-NR⁸R⁹, —(C₀-C₆)alkyl-S(═O)₂NR⁸R⁹, —(C₀-C₆)alkyl-NR⁸—S(═O)₂R⁹, —O—(C₁-C₆)alkyl-S(═O)₂NR⁸R⁹, —O—(C₁-C₆)alkyl-NR⁸—S(═O)₂R⁹, —(C₀-C₆)alkyl-C(═O)—NR⁸R⁹, —(C₀-C₆)alkyl-NR⁸C(═O)—R⁹, —O—(C₁-C₆)alkyl-C(═O)—NR⁸R⁹, —O—(C₁-C₆)alkyl-NR⁸C(═O)—R⁹, —(C₀-C₆)alkyl-OC(═O)—R⁸, —(C₀-C₆)alkyl-C(═O)—OR⁸, —O—(C₁-C₆)alkyl-OC(═O)—R⁸—O—(C₁-C₆)alkyl-C(═O)—OR⁸, —(C₀-C₆)alkyl-C(═O)—R⁸—O—(C₁-C₆)alkyl-C(═O)—R⁸, —(C₀-C₆)alkyl-NR⁸—C(═O)—OR⁹, —(C₀-C₆)alkyl-O—C(═O)—NR⁸R⁹, —(C₀-C₆)alkyl-NR⁸—C(═NR⁹)—NR¹⁰R¹¹, —(C₀-C₆)alkyl-NR⁸—C(═O)—NR⁹R¹⁰ and —(C₀-C₆)alkyl-NR⁸—C(═S)—NR⁹R¹⁰; and
n is an integer ranging from 1 to 3.

All other radicals are defined as in Formula (II).

In a more preferred aspect of Formula (II-a), the invention provides a compound according to Formula (II-a1), 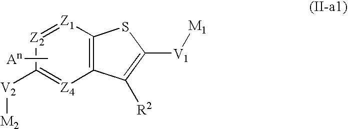
a pharmaceutically acceptable acid or base addition salt thereof, a stereochemically isomeric form thereof and an N-oxide form thereof, wherein:
V₁ and V₂ are each independently selected from the group of a covalent bond, —O—, —C(═O)—, —C(═O)O—, —C(═O)NR¹²—, —S—, —S(O)—, —S(O)₂—, —S(O)₂NR¹², —NR¹²—, —NR¹²C(═O)—, —NR¹²C(═O)NR¹³—, —NR¹²S(O)₂—, —NR¹²C(═S)NR¹³—, —OC(═O)—, —OC(═O)NR¹², —NR¹²C(═O)O—, and an optionally substituted radical selected from the group of —(C₁-C₆)alkyl-, —(C₂-C₆)alkynyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₁-C₆)alkyl-, —(C₀-C₆)alkyl-O—(C₂-C₆)alkynyl-, —(C₀-C₆)alkyl-O—(C₂-C₆)alkenyl-, —(C₀-C₆)alkyl-O—(C₃-C₇)cycloalkyl-, —(C₀-C₆)alkyl-C(═O)—(C₁-C₆)alkyl-, —(C₀-C₆)alkyl-C(═O)—(C₂-C₆)alkynyl-, —(C₀-C₆)alkyl-C(═O)—(C₂-C₆)alkenyl-, —(C₀-C₆)alkyl-C(═O)—(C₄-C₁₀)alkylcycloalkyl-, —(C₀-C₆)alkyl-C(═O)—(C₃-C₇)cycloalkyl-, —(C₀-C₆)alkyl-C(═O)O—(C₁-C₆)alkyl-, —(C₀-C₆)alkyl-C(═O)O—(C₂-C₆)alkynyl-, —(C₀-C₆)alkyl-C(═O)O—(C₂-C₆)alkenyl-, —(C₀-C₆)alkyl-C(═O)O—(C₃-C₇)cycloalkyl-, —(C₀-C₆)alkyl-C(═O)O—(C₄-C₁₀)alkylcycloalkyl-, —(C₀-C₆)alkyl-C(═O)NR¹²—(C₁-C₆)alkyl-, —(C₀-C₆)alkyl-C(═O)NR¹²—(C₂-C₆)alkynyl-, —(C₀-C₆)alkyl-C(═O)NR¹²—(C₂-C₆)alkenyl-, —(C₀-C₆)alkyl-C(═O)NR¹²—(C₃-C₇)cycloalkyl-, —(C₀-C₆)alkyl-C(═O)NR¹²—(C₄-C₁₀)alkylcycloalkyl-, —(C₀-C₆)alkyl-S—(C₁-C₆)alkyl-, —(C₀-C₆)alkyl-S—(C₂-C₆)alkynyl-, —(C₀-C₆)alkyl-S—(C₂-C₆)alkenyl-, —(C₀-C₆)alkyl-S—(C₃-C₇)cycloalkyl-, —(C₀-C₆)alkyl-S—(C₄-C₁₀)alkylcycloalkyl-, —(C₀-C₆)alkyl-S(O)—(C₁-C₆)alkyl-, —(C₀-C₆)alkyl-O—(C₂-C₆)alkynyl-, —(C₀-C₆)alkyl-S(O)—(C₂-C₆)alkenyl-, —(C₀-C₆)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₁-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-S(O)₂NR¹²—(C₁-C₆)alkyl-, —(C₀-C₆)alkyl-S(O)₂NR¹²—(C₂-C₆)alkynyl-, —(C₀-C₆)alkyl-S(O)₂NR¹²—(C₂-C₆)alkenyl-, —(C₀-C₆)alkyl-S(O)₂NR¹²—(C₃-C₇)cycloalkyl-, —(C₀-C₆)alkyl-S(O)₂NR¹²—(C₄-C₁₀)alkylcycloalkyl-, —(C₀-C₆)alkyl-NR¹²—(C₁-C₆)alkyl-, —(C₀-C₆)alkyl-NR²—(C₂-C₆)alkynyl-, —(C₀-C₆)alkyl-N_{—2}-(C₂-C₆)alkenyl-, —(C₀-C₆)alkyl-NR¹²—(C₃-C₇)cycloalkyl-, —(C₀-C₆)alkyl-NR¹²—(C₄-C₁₀)alkylcycloalkyl-, —(C₀-C₆)alkyl-NR¹²C(═O)—(C₁-C₆)alkyl-, —(C₀-C₆)alkyl-NR¹²C(═O)—(C₂-C₆)alkynyl-, —(C₀-C₆)alkyl-NR¹²C(═O)—(C₂-C₆)alkenyl-, —(C₀-C₆)alkyl-NR¹²C(═O)—(C₃-C₇)cycloalkyl-, —(C₀-C₆)alkyl-NR¹²C(═O)—(C₄-C₁₀)alkylcycloalkyl-, —(C₀-C₆)alkyl-NR¹²C(═O)NR¹³—(C₁-C₆)alkyl-, —(C₀-C₆)alkyl-NR¹²C(═O)NR¹³—(C₂-C₆)alkynyl-, —(C₀-C₆)alkyl-NR¹²C(═O)NR¹³—(C₂-C₆)alkenyl-, —(C₀-C₆)alkyl-NR¹²C(═O)NR¹³—(C₃-C₇)cycloalkyl-, —(C₀-C₆)alkyl-NR¹²C(═O)NR¹³—(C₄-C₁₀)alkylcycloalkyl-, —(C₀-C₆)alkyl-NR¹²S(O)₂—(C₁-C₆)alkyl-, —(C₀-C₆)alkyl-NR¹²S(O)₂—(C₂-C₆)alkynyl-, —(C₀-C₆)alkyl-NR¹²S(O)₂—(C₂-C₆)alkenyl-, —(C₀-C₆)alkyl-NR¹²S(O)₂—(C₃-C₇)cycloalkyl- and —(C₀-C₆)alkyl-NR¹²S(O)₂—(C₄-C₁₀)alkylcycloalkyl-.

All other radicals are defined as in Formula (II-a).

In a further preferred aspect of Formula (II-a1), V₁ is a radical selected from the group of —O—, —C(═O)—, —C(═O)O—, —C(═O)NR¹²—, —S—, —S(O)—, —S(O)₂—, —S(O)₂NR¹²—, —NR¹²—, —NR¹²C(═O)—, —NR¹²C(═O)NR¹³—, —NR¹²S(O)₂—, —NR¹²C(═S)NR¹³—, —OC(═O)—, —OC(═O)NR¹², —NR¹²C(═O)O—, and an optionally substituted radical selected from the group of —(C₁-C₆)alkyl-, —(C₂-C₆)alkynyl-, —(C₂-C₆)alkenyl-, —(C₃-C₇)cycloalkyl-, —(C₁-C₆)alkylhalo-, —(C₁-C₆)alkylcyano-, —(C₀-C₆)alkyl-O—(C₁-C₆)alkyl-, —(C₀-C₆)alkyl-O—(C₃-C₇)cycloalkyl-, —(C₀-C₆)alkyl-C(═O)—(C₁-C₆)alkyl-, —(C₀-C₆)alkyl-C(═O)—(C₄-C₁₀)cycloalkyl-, —(C₀-C₆)alkyl-C(═O)O—(C₁-C₆)alkyl-, —(C₀-C₆)alkyl-C(═O)O—(C₃-C₇)cycloalkyl-, —(C₀-C₆)alkyl-C(═O)NR¹²—, —(C₁-C₆)alkyl-, —(C₀-C₆)alkyl-C(═O)NR¹²—(C₃-C₇)cycloalkyl-, —(C₀-C₆)alkyl-S—(C₁-C₆)alkyl-, —(C₀-C₆)alkyl-S—(C₃-C₇)cycloalkyl-, —(C₀-C₆)alkyl-S(O)—(C₁-C₆)alkyl-, —(C₀-C₆)alkyl-S(O)—(C₃-C₇)cycloalkyl-, —(C₀-C₆)alkyl-S(O)₂—(C₁-C₆)alkyl-, —(C₀-C₆)alkyl-S(O)₂—(C₃-C₇)cycloalkyl-, —(C₀-C₆)alkyl-S(O)₂NR¹²—(C₁-C₆)alkyl-, —(C₀-C₆)alkyl-S(O)₂NR¹²—(C₃-C₇)cycloalkyl-, —(C₀-C₆)alkyl-NR¹²—(C₁-C₆)alkyl-, —(C₀-C₆)alkyl-NR¹²—(C₃-C₇)cycloalkyl-, —(C₀-C₆)alkyl-NR¹²C(═O)—(C₁-C₆)alkyl-, —(C₀-C₆)alkyl-NR¹²C(═O)—(C₃-C₇)cycloalkyl-, —(C₀-C₆)alkyl-NR¹²C(═O)NR¹³—(C₁-C₆)alkyl-, —(C₀-C₆)alkyl-NR¹²C(═O)NR¹³—(C₃-C₇)cycloalkyl-, —(C₀-C₆)alkyl-NR¹²S(O)₂—(C₁-C₆)alkyl- and —(C₀-C₆)alkyl-NR¹²S(O)₂—(C₃-C₇)cycloalkyl-.

All other radicals are defined as in Formula (II-a1).

In a second preferred aspect of Formula (II), the invention provides a compound according to Formula (II-b), 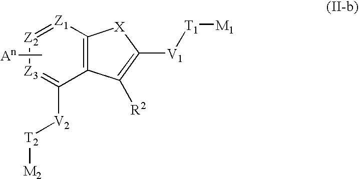
a pharmaceutically acceptable acid or base addition salt thereof, a stereochemically isomeric form thereof and an N-oxide form thereof, wherein:
R² is selected from the group of hydrogen, halo, —CN, —OH, —NO2, —CF₃, —NH₂, —SH, —C(═NR⁴)NR⁵R⁶—C(═O)R⁴, —C(═NR⁴)R⁵, —C(═O)OR⁴, —C(═O)NR⁴R⁵, —SR⁴, —S(O)R⁴, —S(O)₂R⁴, —NR⁴R⁵, —NR⁴C(═O)R⁵, —NR⁴C(═NR⁵)R⁶, —NR⁴C(═NR⁵)NR⁶R⁷, —NR⁴C(═O)OR⁵, —NR⁴C(═O)NR⁵R⁶, —NR⁴S(O)₂R⁵, —S(O)₂NR⁴R⁵, —C(═S)NR⁴R⁵, —OC(═O)R⁴, —OC(═O)NR⁴R⁵, —OR⁴, and an optionally substituted radical selected from the group of —(C₁-C₆)alkyl, —(C₁-C₆)alkylhalo, —(C₂-C₆)alkynyl, —(C₂-C₆)alkenyl, —(C₃-C₇)cycloalkyl, —(C₃-C₈)cycloalkenyl, —(C₁-C₆)alkylcyano, —(C₁-C₆)alkylaryl, —(C₁-C₆)alkylheteroaryl, aryl and heteroaryl;
Aⁿ radicals are each independently selected from the group of hydrogen, halo, —CN, —OH, —NO₂, —CF₃, —SH, —NH₂ and an optionally substituted radical selected from the group of —(C₁-C₆)alkyl, —(C₁-C₆)alkylhalo, —(C₂-C₆)alkynyl, —(C₂-C₆)alkenyl, —(C₃-C₇)cycloalkyl, —(C₁-C₆)alkylcyano, —O—(C₁-C₆)alkyl, —O—(C₁-C₆)alkylhalo, —O—(C₁-C₆)alkylcyano, —O—(C₃-C₆)alkynyl, —O—(C₃-C₇)cycloalkyl, —O—(C₂-C₆)alkenyl, —O—(C₂-C₆)alkyl-OR⁸, —O—(C₁-C₆)alkyl-heteroaryl, —O—(C₀-C₆)alkylaryl, —(C₀-C₆)alkyl-OR⁸, —(C₃-C₇)cycloalkyl-(C₁-C₆)alkyl, —O—(C₃-C₇)cycloalkyl-(C₁-C₆)alkyl, —O-heteroaryl, heteroaryl, —(C₁-C₆)alkyl-heteroaryl, aryl, —O-aryl, —(C₁-C₆)alkylaryl, —(C₁-C₆)alkylhalo-OR⁸, —(C₃-C₆)alkynyl-OR⁸, —(C₃-C₆)alkenyl-OR⁸, —(C₀-C₆)alkyl-SR⁸, —O—(C₂-C₆)alkyl-SR⁸, —(C₁-C₆)alkyl-S(═O)—R⁸, —O—(C₁-C₆)alkyl-S(═O)—R⁸, —(C₀-C₆)alkyl-S(═O)₂—R⁸, —O—(C₁-C₆)alkyl-S(═O)₂—R⁸, —(C₀-C₆)alkyl-NR⁸R⁹, —O—(C₂-C₆)alkyl-NR⁸R⁹, —(C₀-C₆)alkyl-S(═O)₂NR⁸R⁹, —(C₀-C₆)alkyl-NR⁸—S(═O)₂R⁹, —O—(C₁-C₆)alkyl-S(═O)₂NR⁸R⁹, —O—(C₁-C₆)alkyl-NR⁸—S(═O)₂R⁹, —(C₀-C₆)alkyl-C(═O)—NR⁸R⁹, —(C₀-C₆)alkyl-NR⁸C(═O)—R⁹, —O—(C₁-C₆)alkyl-C(═O)—NR⁸R⁹, —O—(C₁-C₆)alkyl-NR⁸C(═O)—R⁹, —(C₀-C₆)alkyl-OC(═O)—R⁸, —(C₀-C₆)alkyl-C(═O)—OR⁸, —O—(C₁-C₆)alkyl-OC(═O)—R⁸, —O—(C₁-C₆)alkyl-C(═O)—OR⁸, —(C₀-C₆)alkyl-C(═O)—R⁸—O—(C₁-C₆)alkyl-C(═O)—R⁸, —(C₀-C₆)alkyl-NR⁸—C(═O)—OR⁹, —(C₀-C₆)alkyl-O—C(═O)—NR⁸R⁹, —(C₀-C₆)alkyl-NR⁸—C(═NR⁹)—NR¹⁰R¹¹, —(C₀-C₆)alkyl-NR⁸—C(═O)—NR⁹R¹⁰ and —(C₀-C₆)alkyl-NR⁸—C(═S)—NR⁹R¹⁰; and
n is an integer ranging from 1 to 3.

All other radicals are defined as in Formula (II).

In a preferred aspect of Formula (II-b), the invention provides a compound according to
Formula (II-b1) 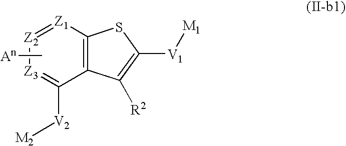
a pharmaceutically acceptable acid or base addition salt thereof, a stereochemically isomeric form thereof and an N-oxide form thereof, wherein:
V₁ and V₂ are each independently selected from the group of a covalent bond, —O—, —C(═O)—, —C(═O)O—, —C(═O)NR¹²—, —S—, —S(O)—, —S(O)₂—, —S(O)₂NR¹²—, —NR¹²—, —NR¹²C(═O)—, —NR¹²C(═O)NR¹³—, —NR¹²S(O)₂—, —NR¹²C(═S)NR¹³—, —OC(═O)—, —OC(═O)NR¹², —NR¹²C(═O)O, and an optionally substituted radical selected from the group of —(C₁-C₆)alkyl-, —(C₂-C₆)alkynyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₁-C₆)alkyl-, —(C₀-C₆)alkyl-O—(C₂-C₆)alkynyl-, —(C₀-C₆)alkyl-O—(C₂-C₆)alkenyl-, —(C₀-C₆)alkyl-O—(C₃-C₇)cycloalkyl-, —(C₀-C₆)alkyl-C(═O)—(C₁-C₆)alkyl-, —(C₀-C₆)alkyl-C(═O)—(C₂-C₆)alkynyl-, —(C₀-C₆)alkyl-C(═O)—(C₂-C₆)alkenyl-, —(C₀-C₆)alkyl-C(═O)—(C₄-C₁₀)alkylcycloalkyl-, —(C₀-C₆)alkyl-C(═O)—(C₃-C₇)cycloalkyl-, —(C₀-C₆)alkyl-C(═O)O—(C₁-C₆)alkyl-, —(C₀-C₆)alkyl-C(═O)O—(C₂-C₆)alkynyl-, —(C₀-C₆)alkyl-C(═O)O—(C₂-C₆)alkenyl-, —(C₀-C₆)alkyl-C(═O)O—(C₃-C₇)cycloalkyl-, —(C₀-C₆)alkyl-C(═O)O—(C₄-C₁₀)alkylcycloalkyl-, —(C₀-C₆)alkyl-C(═O)NR¹²—(C₁-C₆)alkyl-, —(C₀-C₆)alkyl-C(═O)NR¹²—(C₂-C₆)alkynyl-, —(C₀-C₆)alkyl-C(═O)NR¹²—(C₂-C₆)alkenyl-, —(C₀-C₆)alkyl-C(═O)NR¹²—(C₃-C₇)cycloalkyl-, —(C₀-C₆)alkyl-C(═O)NR¹²—(C₄-C₁₀)alkylcycloalkyl-, —(C₀-C₆)alkyl-S—(C₁-C₆)alkyl-, —(C₀-C₆)alkyl-S—(C₂-C₆)alkynyl-, —(C₀-C₆)alkyl-S—(C₂-C₆)alkenyl-, —(C₀-C₆)alkyl-S—(C₃-C₇)cycloalkyl-, —(C₀-C₆)alkyl-S—(C₄-C₁₀)alkylcycloalkyl-, —(C₀-C₆)alkyl-S(O)—(C₁-C₆)alkyl-, —(C₀-C₆)alkyl-O—(C₂-C₆)alkynyl-, —(C₀-C₆)alkyl-S(O)—(C₂-C₆)alkenyl-, —(C₀-C₆)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₁-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-S(O)₂NR¹²—(C₁-C₆)alkyl-, —(C₀-C₆)alkyl-S(O)₂NR¹²—(C₂-C₆)alkynyl-, —(C₀-C₆)alkyl-S(O)₂NR¹²—(C₂-C₆)alkenyl-, —(C₀-C₆)alkyl-S(O)₂NR¹²—(C₃-C₇)cycloalkyl-, —(C₀-C₆)alkyl-S(O)₂NR¹²—(C₄-C₁₀)alkylcycloalkyl-, —(C₀-C₆)alkyl-NR¹²—(C₁-C₆)alkyl-, —(C₀-C₆)alkyl-NR²—(C₂-C₆)alkynyl-, —(C₀-C₆)alkyl-N_{—2}-(C₂-C₆)alkenyl-, —(C₀-C₆)alkyl-NR¹²—(C₃-C₇)cycloalkyl-, —(C₀-C₆)alkyl-NR¹²—(C₄-C₁₀)alkylcycloalkyl-, —(C₀-C₆)alkyl-NR¹²C(═O)—(C₁-C₆)alkyl-, —(C₀-C₆)alkyl-NR¹²C(═O)—(C₂-C₆)alkynyl-, —(C₀-C₆)alkyl-NR¹²C(═O)—(C₂-C₆)alkenyl-, —(C₀-C₆)alkyl-NR¹²C(═O)—(C₃-C₇)cycloalkyl-, —(C₀-C₆)alkyl-NR¹²C(═O)—(C₄-C₁₀)alkylcycloalkyl-, —(C₀-C₆)alkyl-NR¹²C(═O)NR¹³—(C₁-C₆)alkyl-, —(C₀-C₆)alkyl-NR¹²C(═O)NR¹³—(C₂-C₆)alkynyl-, —(C₀-C₆)alkyl-NR¹²C(═O)NR¹³—(C₂-C₆)alkenyl-, —(C₀-C₆)alkyl-NR¹²C(═O)NR¹³—(C₃-C₇)cycloalkyl-, —(C₀-C₆)alkyl-NR¹²C(═O)NR¹³—(C₄-C₁₀)alkylcycloalkyl-, —(C₀-C₆)alkyl-NR¹²S(O)₂—(C₁-C₆)alkyl-, —(C₀-C₆)alkyl-NR¹²S(O)₂—(C₂-C₆)alkynyl-, —(C₀-C₆)alkyl-NR¹²S(O)₂—(C₂-C₆)alkenyl-, —(C₀-C₆)alkyl-NR¹²S(O)₂—(C₃-C₇)cycloalkyl- and —(C₀-C₆)alkyl-NR¹²S(O)₂—(C₄-C₁₀)alkylcycloalkyl-.

All other radicals are defined as in Formula (II-b).

In a further preferred aspect of Formula (II-b1), the invention provides a compound according to Formula (II-b1) wherein:

V₁ is selected from the group of a covalent bond, —O—, —C(═O)—, —C(═O)O—, —C(═O)NR¹²—, —S—, —S(O)—, —S(O)₂—, —S(O)₂NR¹²—, —NR¹²—, —NR¹²C(═O)—, —NR¹²C(═O)NR¹³—, —NR¹²S(O)₂—, —NR¹²C(═S)NR¹³—, —OC(═O)—, —OC(═O)NR¹², —NR¹²C(═O)O—, and an optionally substituted radical selected from the group of —(C₁-C₆)alkyl-, —(C₂-C₆)alkynyl-, —(C₂-C₆)alkenyl-, —(C₃-C₇)cycloalkyl-, —(C₁-C₆)alkylhalo-, —(C₁-C₆)alkylcyano-, —(C₀-C₆)alkyl-O—(C₁-C₆)alkyl-, —(C₀-C₆)alkyl-O—(C₃-C₇)cycloalkyl-, —(C₀-C₆)alkyl-C(═O)—(C₁-C₆)alkyl-, —(C₀-C₆)alkyl-C(═O)—(C₄-C₁₀)cycloalkyl-, —(C₀-C₆)alkyl-C(═O)O—(C₁-C₆)alkyl-, —(C₀-C₆)alkyl-C(═O)O—(C₃-C₇)cycloalkyl-, —(C₀-C₆)alkyl-C(═O)NR¹²—, —(C₁-C₆)alkyl-, —(C₀-C₆)alkyl-C(═O)NR¹²—(C₃-C₇)cycloalkyl-, —(C₀-C₆)alkyl-S—(C₁-C₆)alkyl-, —(C₀-C₆)alkyl-S—(C₃-C₇)cycloalkyl-, —(C₀-C₆)alkyl-S(O)—(C₁-C₆)alkyl-, —(C₀-C₆)alkyl-S(O)—(C₃-C₇)cycloalkyl-, —(C₀-C₆)alkyl-S(O)₂—(C₁-C₆)alkyl-, —(C₀-C₆)alkyl-S(O)₂—(C₃-C₇)cycloalkyl-, —(C₀-C₆)alkyl-S(O)₂NR¹²—(C₁-C₆)alkyl-, —(C₀-C₆)alkyl-S(O)₂NR¹²—(C₃-C₇)cycloalkyl-, —(C₀-C₆)alkyl-NR¹²—(C₁-C₆)alkyl-, —(C₀-C₆)alkyl-NR¹²—(C₃-C₇)cycloalkyl-, —(C₀-C₆)alkyl-NR¹²C(═O)—(C₁-C₆)alkyl-, —(C₀-C₆)alkyl-NR¹²C(═O)—(C₃-C₇)cycloalkyl-, —(C₀-C₆)alkyl-NR¹²C(═O)NR¹³—(C₁-C₆)alkyl-, —(C₀-C₆)alkyl-NR¹²C(═O)NR¹³—(C₃-C₇)cycloalkyl-, —(C₀-C₆)alkyl-NR¹²S(O)₂—(C₁-C₆)alkyl- and —(C₀-C₆)alkyl-NR¹²S(O)₂—(C₃-C₇)cycloalkyl-; and

M₂ is an optionally substituted 3 to 10 membered ring selected from the group of aryl, heteroaryl, heterocyclic and cycloalkyl rings.

All other radicals are defined as in Formula (II-b1).

In a further preferred aspect of Formula (II-b1), the invention provides a compound of Formula (II-b2) 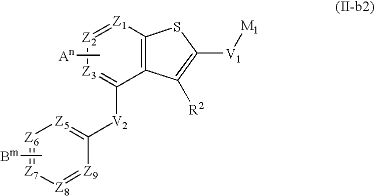
a pharmaceutically acceptable acid or base addition salt thereof, a stereochemically isomeric form thereof and an N-oxide form thereof, wherein:
Z₅, Z₆, Z₇, Z₈ and Z₉ are each independently selected from a covalent bond, C, S, N and O, with the provision that a 5 or 6 membered heteroaryl or aryl ring is formed, which may further be substituted by 1 to 5 radicals B^{m};
B^{m} radicals are each independently selected from the group of hydrogen, halo, —CN, —OH, —NO2, —CF₃, —SH, —NH₂, and an optionally substituted radical selected from the group of —(C₁-C₆)alkyl, —(C₁-C₆)alkylhalo, —(C₂-C₆)alkynyl, —(C₂-C₆)alkenyl, —(C₃-C₇)cycloalkyl, —(C₁-C₆)alkylcyano, —O—(C₁-C₆)alkyl, —O—(C₁-C₆)alkylhalo, —O—(C₁-C₆)alkylcyano, —O—(C₃-C₆)alkynyl, —O—(C₃-C₇)cycloalkyl, —O—(C₂-C₆)alkenyl, —O—(C₂-C₆)alkyl-OR²², —(C₀-C₆)alkyl-OR²², —O-heteroaryl, heteroaryl, —(C₃-C₆)alkynyl-OR²², —(C₃-C₆)alkenyl-OR²², —(C₀-C₆)alkyl-S—R²², —(C₀-C₆)alkyl-NR²²R²³, —O—(C₂-C₆)alkyl-NR²²R²³, —(C₀-C₆)alkyl-S(═O)₂NR²²R²³, —(C₀-C₆)alkyl-NR²²—S(═O)₂R²³, —O—(C₁-C₆)alkyl-S(═O)₂NR²²R²³, —O—(C₁-C₆)alkyl-NR²²—S(═O)₂R²³, —(C₀-C₆)alkyl-C(═O)—NR²²R²³, —(C₀-C₆)alkyl-NR²²C(═O)—R²³, —O—(C₁-C₆)alkyl-C(═O)—NR²²R²³, —O—(C₁-C₆)alkyl-NR²²C(═O)—R²³, —(C₀-C₆)alkyl-OC(═O)—R²², —(C₀-C₆)alkyl-C(═O)—OR²², —O—(C₁-C₆)alkyl-OC(═O)—R²², —O—(C₁-C₆)alkyl-C(═O)—OR²², —(C₀-C₆)alkyl-C(═O)—R²² and —O—(C₁-C₆)alkyl-C(═O)—R²²;
m is an integer ranging from 1 to 5;
R²² and R²³ are each independently hydrogen or an optionally substituted radical selected from the group of —(C₁-C₆)alkylhalo, —(C₁-C₆)alkyl, —(C₁-C₆)alkylcyano, —(C₂-C₆)alkynyl, —(C₂-C₆)alkenyl, —(C₃-C₇)cycloalkyl, —(C₄-C₁₀)alkylcycloalkyl, heteroaryl, —(C₁-C₆)alkylheteroaryl, aryl, —(C₁-C₆)alkylaryl, —(C₂-C₆)alkynyl-(C₃-C₇)cycloalkyl, —(C₂-C₆)alkynyl-heteroaryl, —(C₂-C₆)alkynyl-aryl, —(C₂-C₆)alkenyl-(C₃-C₇)cycloalkyl, —(C₂-C₆)alkenyl-heteroaryl and —(C₂-C₆)alkenyl-aryl;
Z₁, Z₂ and Z₃ are each independently selected from C and N, provided that at least 1 nitrogen is present;
V₁ and V₂ are each independently selected from the group of a covalent bond, —C(═O)—, and an optionally substituted radical selected from the group of —(C₁-C₆)alkyl, —(C₂-C₆)alkynyl, —(C₂-C₆)alkenyl, —(C₃-C₇)cycloalkyl, —(C₁-C₆)alkylhalo, —(C₀-C₆)alkyl-C(═O)—(C₀-C₆)alkyl, —(C₀-C₆)alkyl-C(═O)NR⁷—(C₀-C₆)alkyl, —(C₀-C₆)alkyl-O—(C₀-C₆)alkyl, —CO—C₆)alkyl-S—(C₀-C₆)alkyl, —(C₀-C₆)alkyl-S(O)₂—(C₀-C₆)alkyl, —(C₀-C₆)alkyl-S(O)₂NR⁷—(C₀-C₆)alkyl, —(C₀-C₆)alkyl-NR⁷—(C₀-C₆)alkyl, —(C₀-C₆)alkyl-NR⁷C(═O)-(C₁-C₆)alkyl and —(C₀-C₆)alkyl-NR⁷S(O)₂—(C₀-C₆)alkyl;
R⁷ is hydrogen or an optionally substituted radical selected from the group of —(C₁-C₆)alkyl, —(C₁-C₆)alkylhalo, —(C₂-C₆)alkynyl, —(C₂-C₆)alkenyl, —(C₃-C₇)cycloalkyl and —(C₁-C₆)alkylcyano; and
Aⁿ is selected from the group of hydrogen, halo, —CN, —OH, —NO2, —CF₃, —NH₂, and an optionally substituted radical selected from the group of —(C₁-C₆)alkyl, —(C₁-C₆)alkylhalo, —(C₂-C₆)alkynyl, —(C₂-C₆)alkenyl, —(C₃-C₇)cycloalkyl, —(C₁-C₆)alkylcyano, —O—(C₁-C₆)alkyl, —O—(C₁-C₆)alkylhalo, —O—(C₁-C₆)alkylcyano, —O—(C₃-C₆)alkynyl, —O—(C₃-C₇)cycloalkyl, —O—(C₂-C₆)alkenyl, —O—(C₂-C₆)alkyl-OR⁸, —(C₀-C₆)alkyl-OR⁸, —O-heteroaryl, —(C₀-C₆)alkyl-SR⁸, —(C₀-C₆)alkyl-S(═O)₂R⁸, —O—(C₁-C₆)alkyl-S(═O)₂R⁸, —(C₀-C₆)alkyl-NR⁸R⁹, —(C₀-C₃)alkyl-O—(C₂-C₆)alkyl-NR⁸R⁹, —(C₀-C₆)alkyl-C(═O)—NR⁸R⁹, —(C₀-C₆)alkyl-NR⁸C(═O)—R⁹, —(C₀-C₆)alkyl-C(═O)—R⁸ and —O—(C₁-C₆)alkyl-C(═O)—R⁸.

All other radicals are defined as in Formula (II-b1).

In a further preferred aspect of Formula (II-b2), the invention provides a compound according to Formula (II-b2), wherein:

Z₁, Z₂, and Z₃ are each independently selected from C and N, provided that at least two nitrogens are present;

V₁ may be selected from the group of a covalent bond, —C(═O)—, and an optionally substituted radical selected from the group of —(C₁-C₆)alkyl-, —(C₀-C₆)alkyl-O—(C₁-C₆)alkyl-, —(C₀-C₆)alkyl-S—(C₁-C₆)alkyl- and —(C₀-C₆)alkyl-NR¹²—(C₁-C₆)alkyl-optionally substituted by one or more radicals from the group of —OCH₃, —OCF₃, —CF₃, —F and —CN;

V₂ is an optionally substituted radical selected from the group of —(C₁-C₆)alkyl, —(C₂-C₆)alkynyl, —(C₂-C₆)alkenyl, —(C₃-C₇)cycloalkyl, —(C₁-C₆)alkylhalo, —(C₀-C₆)alkyl-C(═O)—(C₀-C₆)alkyl, —(C₀-C₆)alkyl-C(═O)NR⁷—(C₀-C₆)alkyl, —(C₀-C₆)alkyl-O—(C₀-C₆)alkyl, —(C₀-C₆)alkyl-S—(C₀-C₆)alkyl, —(C₀-C₆)alkyl-S(O)₂—(C₀-C₆)alkyl, —(C₀-C₆)alkyl-S(O)₂NR⁷—(C₀-C₆)alkyl, —(C₀-C₆)alkyl-NR⁷—(C₀-C₆)alkyl, —(C₀-C₆)alkyl-NR⁷C(═O)—(C₀-C₆)alkyl and —(C₀-C₆)alkyl-NR⁷S(O)₂—(C₀-C₆)alkyl;

R² is selected from the group of hydrogen, halo, —OCH₃, —OCF₃, —CF₃, and a linear (C₁-C₆)alkyl radical, optionally substituted by —CN, —OCH₃, —OCF₃, —CF₃ or halo;

Aⁿ is selected from the group of hydrogen, halo, —CN, —OH, —CF₃, —NH₂, and an optionally substituted radical selected from the group of —(C₁-C₆)alkyl, —(C₁-C₆)alkylhalo, —(C₂-C₆)alkynyl, —(C₂-C₆)alkenyl, —(C₃-C₇)cycloalkyl, —(C₁-C₆)alkylcyano, —O—(C₁-C₆)alkyl, —O—(C₁-C₆)alkylhalo, —O—(C₁-C₆)alkylcyano, —O—(C₃-C₆)alkynyl, —O—(C₃-C₇)cycloalkyl, —O—(C₂-C₆)alkenyl, —O—(C₂-C₆)alkyl-OR¹⁸, —(C₀-C₆)alkyl-OR¹⁸, —(C₀-C₆)alkyl-NR¹⁸R¹⁹ and —(C₀-C₃)alkyl-O—(C₂-C₆)alkyl-NR¹⁸R¹⁹; and the radical 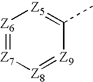
is selected from the group of aryl, thienyl, pyridyl, thiazolyl, isothiazolyl, oxazolyl, isoxazolyl and pyrimidinyl, each radical optionally substituted by m B^{m} radicals.

All other radicals are defined as in Formula (II-b2).

In a further preferred aspect of the invention, the invention provides a compound according to Formula (I), wherein:

X is —S—;

R¹ is —(C₁-C₆)alkyl or a radical V₁-T₁-M₁;

Z₁, Z₂, Z₃ and Z₄ are each independently selected from C and N; with the provision that a 6-membered heteroaryl ring is formed, which is substituted with n radicals Aⁿ;

Aⁿ radicals are each independently selected from the group of hydrogen, halo, —(C₁-C₆)-alkyl, —O—(C₁-C₆)alkyl, —(C₀-C₆)alkyl-NR⁸R⁹, and a radical V2-T2-M2;

n is an integer ranging from 1 to 2;

T₁ and T₂ are each a covalent bond;

V₁ and V₂ are each independently selected from the group of a covalent bond, —C(═O)—, and an optionally substituted radical selected from the group of —(C₁-C₆)alkyl-, —(C₀-C₆)alkyl-S—(C₁-C₆)alkyl- and —(C₀-C₆)alkyl-NR¹²—(C₁-C₆)alkyl-, wherein R¹² is hydrogen or —(C₁-C₆)alkyl optionally substituted with hydroxy;

M₁ and M₂ are each independently selected from the group of hydrogen, —CN, —OH, —NR¹⁵R¹⁶, —OR¹⁵, and an optionally substituted 6 membered ring selected from the group of aryl and heteroaryl;

R⁸, R⁹, R¹², R¹⁵ and R¹⁶ are each independently hydrogen or an optionally substituted radical selected from the group of —(C₁-C₆)alkyl and aryl; aryl is phenyl; and

wherein the optional substitution refers to one or more substituents selected from the group of hydroxy; (C₁-C₆)alkyloxy, aryl, heterocycle, halo, trifluoromethyl, amino, mono- and di-((C₁-C₆)alkylcarbonyl)amino, (C₁-C₆)alkylsulfonyl and aminosulfonyl.

In a further preferred aspect of the invention, the invention provides a compound according to Formula (I), wherein:

X is —S—;

Z₁ is N, Z₂ is C, Z₃ is N or C, and Z₄ is C;

A is selected from the group of hydrogen; halo; —(C₁-C₆)alkyl; —O—(C₁-C₆)alkyl and —(C₀-C₆)alkyl-NR⁸R⁹ wherein R⁸ and R⁹ are each independently hydrogen or —(C₁-C₆)-alkyl;

n is an integer, equal to 0, 1 or 2;

R¹ is —(C₁-C₆)alkyl or a radical V₁-T₁-M₁;

T₁ is a covalent bond;

V₁ is selected from the group of a covalent bond; —C(═O)— and —(C₁-C₆)alkyl-, more in particular —CH₂—, —CH₂CH₂—, —CH₂CH₂CH₂—, —CH(CH₃)CH₂—, —CH₂CH₂CH₂CH₂— and —CH₂CH(CH₃)CH₂—, each of the alkyl radicals optionally substituted with hydroxy;

M₁ is selected from the group of hydrogen; —OH; —NR¹⁵R¹⁶ wherein R¹⁵ and R¹⁶ are each independently hydrogen or —(C₁-C₆)alkyl; —OR¹⁵, wherein R¹⁵ is —(C₁-C₆)alkyl; and phenyl

V₂ is selected from the group of a covalent bond; —(C₀-C₆)alkyl-NR¹²—(C₁-C₆)alkyl-, wherein R¹² is hydrogen or —(C₁-C₆)alkyl optionally substituted with hydroxy; and —(C₀-C₆)alkyl-S—(C₁-C₆)alkyl-; and

M₂ is selected from the group of phenyl; —CN; benzopiperidinyl; pyridinyl; thienyl; piperidinyl; furyl; OR¹⁵ wherein R¹⁵ is phenyl or —(C₁-C₆)alkyl; —NR¹⁵R¹⁶ wherein R¹⁵ and R¹⁶ are each independently hydrogen or phenyl; —C(═O)R¹⁵ wherein R¹⁵ is phenyl and wherein each alkyl- and phenyl-moiety is optionally substituted with one or two radicals selected from the group of methoxy, ethoxy, chloro, fluoro, phenyl, methyl, ethyl, trifluoromethyl, hydroxy, amino, methylcarbonylamino, methylsulfonyl, aminosulfonyl, tetrazolyl, tetrazolyl(C₁-C₆)alkyl and tetrazolyl(C₁-C₆)alkyloxy.

Particular preferred compounds of the invention are compounds as mentioned in the following list (List of Particular Preferred Compounds), as well as a pharmaceutically acceptable acid or base addition salt thereof, a stereochemically isomeric form thereof and an N-oxide form thereof:
N-benzyl-6-ethylthieno[2,3-d]pyrimidin-4-amine
N-(3,4-dimethoxyphenethyl)-2,6-dimethylthieno[2,3-d]pyrimidin-4-amine
N-(3,4-dimethoxyphenethyl)-6-methylthieno[2,3-d]pyrimidin-4-amine
N-(3,4-dimethoxyphenethyl)-6-ethylthieno[2,3-d]pyrimidin-4-amine
N-(4-methoxyphenethyl)-6-methylthieno[2,3-d]pyrimidin-4-amine
N-(4-methoxyphenethyl)-2,6-dimethylthieno[2,3-d]pyrimidin-4-amine
N-(4-methoxyphenethyl)-2-ethyl-6-methylthieno[2,3-d]pyrimidin-4-amine
N-(3,4-dimethoxyphenethyl)-2-ethyl-6-methylthieno[2,3-d]pyrimidin-4-amine
N-(4-methoxyphenethyl)thieno[2,3-d]pyrimidin-4-amine
6-ethyl-N-(1-phenylethyl)thieno[2,3-d]pyrimidin-4-amine
N-(3-methoxybenzyl)-6-ethylthieno[2,3-d]pyrimidin-4-amine
N-(4-fluorobenzyl)-6-ethylthieno[2,3-d]pyrimidin-4-amine
N-(3-methoxyphenethyl)-2-ethyl-6-methylthieno[2,3-d]pyrimidin-4-amine
N-(3-methoxybenzyl)-2-ethyl-6-methylthieno[2,3-d]pyrimidin-4-amine
N-(4-methoxyphenethyl)-6-benzyl-2-methylthieno[2,3-d]pyrimidin-4-amine
N-(3-methoxybenzyl)-6-benzyl-2-methylthieno[2,3-d]pyrimidin-4-amine
N-(3-chlorobenzyl)-2,6-dimethylthieno[2,3-d]pyrimidin-4-amine
N-(3,4-dimethoxybenzyl)-2,6-dimethylthieno[2,3-d]pyrimidin-4-amine
6-ethyl-N-phenethylthieno[2,3-d]pyrimidin-4-amine
6-ethyl-N-(3-phenylpropyl)thieno[2,3-d]pyrimidin-4-amine
N-(4-methoxyphenethyl)-6-ethyl-2-methylthieno[2,3-d]pyrimidin-4-amine
N-(3,4-dimethoxyphenethyl)-6-ethyl-2-methylthieno[2,3-d]pyrimidin-4-amine
N-(4-chlorophenethyl)-2,6-dimethylthieno[2,3-d]pyrimidin-4-amine
2,6-dimethyl-N-(2-(pyridin-2-yl)ethyl)thieno[2,3-d]pyrimidin-4-amine
N-(4-fluorophenethyl)-2,6-dimethylthieno[2,3-d]pyrimidin-4-amine
N-(4-methylphenethyl)-2,6-dimethylthieno[2,3-d]pyrimidin-4-amine
N-benzyl-2,6-dimethylthieno[2,3-d]pyrimidin-4-amine
N-(3-methoxybenzyl)-2,6-dimethylthieno[2,3-d]pyrimidin-4-amine
N-(3-(trifluoromethyl)phenethyl)-2,6-dimethylthieno[2,3-d]pyrimidin-4-amine
N-(4-methoxyphenethyl)-N,2,6-trimethylthieno[2,3-d]pyrimidin-4-amine
N-(4-methoxyphenethyl)-6-propylthieno[2,3-d]pyrimidin-4-amine
N-phenethyl-6-propylthieno[2,3-d]pyrimidin-4-amine
N-(3-methoxybenzyl)-6-propylthieno[2,3-d]pyrimidin-4-amine
N-(4-methoxyphenethyl)-6-isopropylthieno[2,3-d]pyrimidin-4-amine
6-isopropyl-N-phenethylthieno[2,3-d]pyrimidin-4-amine
N-(3-methoxypropyl)-2,6-dimethylthieno[2,3-d]pyrimidin-4-amine
N-(3-methoxybenzyl)-6-isopropylthieno[2,3-d]pyrimidin-4-amine
6-ethyl-N-(furan-2-ylmethyl)thieno[2,3-d]pyrimidin-4-amine
6-ethyl-4-(3-methylpiperidin-1-yl)thieno[2,3-d]pyrimidine
N-(4-methoxyphenethyl)-6-ethylthieno[2,3-d]pyrimidin-4-amine
N-(3-methoxyphenethyl)-6-ethylthieno[2,3-d]pyrimidin-4-amine
N-(2-methoxyphenethyl)-2,6-dimethylthieno[2,3-d]pyrimidin-4-amine
N-(3-methoxyphenethyl)-2,6-dimethylthieno[2,3-d]pyrimidin-4-amine
2,6-dimethyl-N-phenethylthieno[2,3-d]pyrimidin-4-amine
2,6-dimethyl-N-(2-phenylpropyl)thieno[2,3-d]pyrimidin-4-amine
2-(6-ethylthieno[2,3-d]pyrimidin-4-ylthio)acetonitrile
4-(2-(2,6-dimethylthieno[2,3-d]pyrimidin-4-ylamino)ethyl)phenol
2-(2,6-dimethylthieno[2,3-d]pyrimidin-4-ylamino)-1-phenylethanol
N-(3-(4-methoxyphenyl)propyl)-2,6-dimethylthieno[2,3-d]pyrimidin-4-amine
N-(3-(3-methoxyphenyl)propyl)-2,6-dimethylthieno[2,3-d]pyrimidin-4-amine
2-(2,6-dimethylthieno[2,3-d]pyrimidin-4-yl)-1,2,3,4-tetrahydroisoquinoline
6-butyl-N-phenethylthieno[2,3-d]pyrimidin-4-amine
2-ethyl-N-phenethylthieno[2,3-d]pyrimidin-4-amine
N-(4-aminophenethyl)-2,6-dimethylthieno[2,3-d]pyrimidin-4-amine
4-(2-(6-propylthieno[2,3-d]pyrimidin-4-ylamino)ethyl)phenol
N-(3,4-dimethoxyphenethyl)-6-propylthieno[2,3-d]pyrimidin-4-amine
2-methyl-N-phenethylthieno[2,3-d]pyrimidin-4-amine
2-ethyl-N-phenethylthieno[2,3-b]pyridin-4-amine
2-chloro-6-methyl-N-phenethylthieno[2,3-d]pyrimidin-4-amine
2,6-dimethyl-N-(4-phenylbutyl)thieno[2,3-d]pyrimidin-4-amine
2,6-dimethyl-N-(2-phenoxyethyl)thieno[2,3-d]pyrimidin-4-amine
2-methoxy-6-methyl-N-phenethylthieno[2,3-d]pyrimidin-4-amine
N2,N2,6-trimethyl-N4-phenethylthieno[2,3-d]pyrimidine-2,4-diamine
N-(4-methoxybenzyl)-2,6-dimethylthieno[2,3-d]pyrimidin-4-amine
N-(2-chlorophenethyl)-2,6-dimethylthieno[2,3-d]pyrimidin-4-amine
N-(3-fluorophenethyl)-2,6-dimethylthieno[2,3-d]pyrimidin-4-amine
N-(3-methylphenethyl)-2,6-dimethylthieno[2,3-d]pyrimidin-4-amine
N-(2-methylphenethyl)-2,6-dimethylthieno[2,3-d]pyrimidin-4-amine
N-(4-ethylphenethyl)-2,6-dimethylthieno[2,3-d]pyrimidin-4-amine
N-(3-chlorophenethyl)-2,6-dimethylthieno[2,3-d]pyrimidin-4-amine
N-(3-fluorophenethyl)-2,6-dimethylthieno[2,3-d]pyrimidin-4-amine
N-(3,5-dimethoxyphenethyl)-2,6-dimethylthieno[2,3-d]pyrimidin-4-amine
N-(4-ethoxyphenethyl)-2,6-dimethylthieno[2,3-d]pyrimidin-4-amine
2,6-dimethyl-N-(2-(thiophen-2-yl)ethyl)thieno[2,3-d]pyrimidin-4-amine
N-(4-(methylsulfonyl)phenethyl)-2,6-dimethylthieno[2,3-d]pyrimidin-4-amine
2,6-dimethyl-N-(2-(pyridin-3-yl)ethyl)thieno[2,3-d]pyrimidin-4-amine
N-(3-hydroxyphenethyl)-2,6-dimethylthieno[2,3-d]pyrimidin-4-amine
4-(2-(2,6-dimethylthieno[2,3-d]pyrimidin-4-ylamino)ethyl)benzenesulfonamide
N-(4-phenyl)benzyl-2,6-dimethylthieno[2,3-d]pyrimidin-4-amine
N-(4-(2-(2,6-dimethylthieno[2,3-d]pyrimidin-4-ylamino)ethyl)phenyl)acetamide
(4-(phenethylamino)thieno[2,3-d]pyrimidin-6-yl)methanol
N,N-dimethyl-4-(phenethylamino)thieno[2,3-d]pyrimidine-6-carboxamide
1-(4-(phenethylamino)thieno[2,3-d]pyrimidin-6-yl)propan-1-ol
N-(4-((2H-tetrazol-5-yl)methoxy)phenethyl)-2,6-dimethylthieno[2,3-d]pyrimidin-4-amine
2-(2,6-dimethylthieno[2,3-d]pyrimidin-4-ylamino)-1-phenylethanone
N-(2-(phenylamino)ethyl)-6-propylthieno[2,3-d]pyrimidin-4-amine
4-(2-(2,6-dimethylthieno[2,3-d]pyrimidin-4-ylamino)ethyl)-2-methoxyphenol
4-(2-(2-chloro-6-propylthieno[2,3-d]pyrimidin-4-ylamino)ethyl)phenol
6-isobutyl-N-phenethylthieno[2,3-d]pyrimidin-4-amine
2-ethoxy-N-phenethyl-6-propylthieno[2,3-d]pyrimidin-4-amine
2-ethoxy-N-phenethyl-6-propylthieno[2,3-d]pyrimidin-4-amine
N-(4-methoxyphenethyl)-2-methoxy-6-propylthieno[2,3-d]pyrimidin-4-amine
N-(3-methoxybenzyl)-2-ethoxy-6-propylthieno[2,3-d]pyrimidin-4-amine
4-(2-(2-methoxy-6-propylthieno[2,3-d]pyrimidin-4-ylamino)ethyl)phenol
N2-methyl-N4-phenethyl-6-propylthieno[2,3-d]pyrimidine-2,4-diamine, and
N-(4-((1H-tetrazol-5-yl)methoxy)phenethyl)-2-methoxy-6-propylthieno[2,3-d]pyrimidin-4-amine
   Definition of Terms

Listed below are definitions of various terms used in the specification and claims to describe the present invention.

For the avoidance of doubt it is to be understood that in this specification “(C₁-C₆)” means a carbon radical having 1, 2, 3, 4, 5 or 6 carbon atoms. “(C₀-C₆)” means a carbon radical having 0, 1, 2, 3, 4, 5 or 6 carbon atoms. In this specification “C” means a carbon atom, “N” means a nitrogen atom and “S” means a sulphur atom.

In the case where a subscript is the integer 0 (zero) the radical to which the subscript refers, indicates that the radical is absent, i.e. there is a direct bond between the radicals.

When two or more bonds are adjacent to one another, they are assumed to be equal to one bond. For example, a radical -A-B-, wherein both A and B may be a bond, the radical is depicting a single bond.

In this specification, unless stated otherwise, the term “bond” refers to a saturated covalent bond.

In this specification, unless stated otherwise, the term “alkyl” includes both straight and branched chain alkyl radicals and may be methyl, ethyl, n-propyl, i-propyl, n-butyl, i-butyl, s-butyl, t-butyl, n-pentyl, i-pentyl, t-pentyl, neo-pentyl, n-hexyl or i-hexyl, t-hexyl. The term “(C₀-C₃)alkyl” refers to an alkyl radical having 0, 1, 2 or 3 carbon atoms, and may be methyl, ethyl, n-propyl and i-propyl.

In this specification, unless stated otherwise, the term “cycloalkyl” refers to an optionally substituted carbocycle containing no heteroatoms, including mono-, bi-, and tricyclic saturated carbocycles, as well as fused ring systems. Such fused ring systems can include one ring that is partially or fully unsaturated such as a benzene ring to form fused ring systems such as benzo-fused carbocycles. Cycloalkyl includes such fused ring systems as spirofused ring systems. Examples of cycloalkyl include cyclopropyl, cyclobutyl, cyclopentyl, cyclohexyl, decahydronaphthalene, adamantane, indanyl, fluorenyl, 1,2,3,4-tetrahydronaphthalene and the like. The term “(C₃-C₇)cycloalkyl” may be cyclopropyl, cyclobutyl, cyclopentyl, cyclohexyl, cycloheptyl and the like.

In this specification, unless stated otherwise, the term “alkenyl” includes both straight and branched chain alkenyl radicals. The term “(C₂-C₆)alkenyl” refers to an alkenyl radical having 2 to 6 carbon atoms and one or two double bonds, and may be, but is not limited to vinyl, allyl, propenyl, i-propenyl, butenyl, i-butenyl, crotyl, pentenyl, i-pentenyl and hexenyl.

In this specification, unless stated otherwise, the term “alkynyl” includes both straight and branched chain alkynyl radicals. The term (C₂-C₆)alkynyl having 2 to 6 carbon atoms and one or two triple bonds, and may be, but is not limited to ethynyl, propargyl, butynyl, ibutynyl, pentynyl, i-pentynyl and hexynyl.

The term “aryl” refers to an optionally substituted monocyclic or bicyclic hydrocarbon ring system containing at least one unsaturated aromatic ring. Examples and suitable values of the term “aryl” are phenyl, naphtyl, 1,2,3,4-tetrahydronaphthyl, indyl indenyl and the like.

In this specification, unless stated otherwise, the term “heteroaryl” refers to an optionally substituted monocyclic or bicyclic unsaturated, aromatic ring system containing at least one heteroatom selected independently from N, O or S. Examples of “heteroaryl” may be, but are not limited to thiophene, thienyl, pyridyl, thiazolyl, isothiazolyl, furyl, pyrrolyl, triazolyl, imidazolyl, oxadiazolyl, oxazolyl, isoxazolyl, pyrazolyl, imidazolonyl, oxazolonyl, thiazolonyl, tetrazolyl and thiadiazolyl, benzoimidazolyl, benzooxazolyl, benzothiazolyl, tetrahydrotriazolopyridyl, tetrahydrotriazolopyrimidinyl, benzofuryl, thionaphtyl, indolyl, isoindolyl, pyridonyl, pyridazinyl, pyrazinyl, pyrimidinyl, quinolyl, phthalazinyl, naphthyridinyl, quinoxalinyl, quinazolyl, imidazopyridyl, oxazolopyridyl, thiazolopyridyl, pyridyl, imidazopyridazinyl, oxazolopyridazinyl, thiazolopyridazinyl, cynnolyl, pteridinyl, furazanyl, benzotriazolyl, pyrazolopyridinyl, purinyl and the like.

In this specification, unless stated otherwise, the term “alkylaryl”, “alkylheteroaryl” and “alkylcycloalkyl” refers respectively to a substituent that is attached via the alkyl radical to an aryl, heteroaryl or cycloalkyl radical, respectively. The term “(C₁-C₆)alkylaryl” includes aryl-C₁-C₆-alkyl radicals such as benzyl, 1-phenylethyl, 2-phenylethyl, 1-phenylpropyl, 2-phenylpropyl, 3-phenylpropyl, 1-naphtylmethy, 2-naphthylmethyl, or the like. The term “(C₁-C₆)alkyheteroaryl” includes heteroaryl-C₁-C₃-alkyl radicals, wherein examples of heteroaryl are the same as those illustrated in the above definition, such as 2-furylmethyl, 3-furylmethyl, 2-thienylmethyl, 3-thienylmethyl, 1-imidazolylmethyl, 2-imidazolylmethyl, 2-thiazolylmethyl, 2-pyridylmethyl, 3-pyridylmethyl, 1-quinolylmethyl, or the like.

In this specification, unless stated otherwise, the term “heterocycle” refers to an optionally substituted, monocyclic or bicyclic saturated, partially saturated or unsaturated ring system containing at least one heteroatom selected independently from N, O and S.

In this specification, unless stated otherwise, a 5- or 6-membered ring containing one or more atoms independently selected from C, N, O and S, includes aromatic and heteroaromatic rings as well as carbocyclic and heterocyclic rings which may be saturated or unsaturated. Examples of such rings may be, but are not limited to, furyl, isoxazolyl, isothiazolyl, oxazolyl, pyrazinyl, pyrazolyl, pyridazinyl, pyridyl, pyrimidyl, pyrrolyl, thiazolyl, thienyl, imidazolyl, imidazolidinyl, imidazolinyl, triazolyl, morpholinyl, piperazinyl, piperidyl, piperidonyl, pyrazolidinyl, pyrazolinyl, pyrrolidinyl, pyrrolinyl, tetrahydropyranyl, thiomorpholinyl, phenyl, cyclohexyl, cyclopentyl, cyclohexenyl, and the like.

In this specification, unless stated otherwise, a 3- to 10-membered ring containing one or more atoms independently selected from C, N, O and S, includes aromatic and heteroaromatic rings as well as carbocyclic and heterocyclic rings which may be saturated or unsaturated. Examples of such rings may be, but are not limited to imidazolidinyl, imidazolinyl, morpholinyl, piperazinyl, piperidyl, piperidonyl, pyrazolidinyl, pyrazolinyl, pyrrolidinyl, pyrrolinyl, tetrahydropyranyl, thiomorpholinyl, tetrahydrothiopyranyl, furyl, pyrrolyl, isoxazolyl, isothiazolyl, oxazolyl, oxazolidinonyl, pyrazinyl, pyrazolyl, pyridazinyl, pyridyl, pyrimidyl, pyrrolyl, thiazolyl, thienyl, imidazolyl, triazolyl, phenyl, cyclopropyl, aziridinyl, cyclobutyl, azetidinyl, cyclopentyl, cyclopentenyl, cyclohexyl, cyclohexenyl, cycloheptyl, cycloheptenyl, cyclooctyl, cyclooctenyl, and the like.

In this specification, unless stated otherwise, the term “halo” may be fluoro, chloro, bromo or iodo.

In this specification, unless stated otherwise, the term “alkylhalo” means an alkyl radical as defined above, substituted with one or more halo radicals. The term “(C₁-C₆)alkylhalo” may include, but is not limited to, fluoromethyl, difluoromethyl, trifluoromethyl, fluoroethyl and difluoroethyl. The term “O—C₁-C₆-alkylhalo” may include, but is not limited to, fluoromethoxy, difluoromethoxy, trifluoromethoxy and fluoroethoxy.

In this specification, unless stated otherwise, the term “alkylcyano” means an alkyl radical as defined above, substituted with one or more cyano.

In this specification, unless stated otherwise, the term “optionally substituted” refers to radicals further bearing one or more substituents which may be, but are not limited to, hydroxy, (C₁-C₆)alkyloxy, mercapto, aryl, heterocycle, halo, trifluoromethyl, pentafluoroethyl, cyano, cyanomethyl, nitro, amino, amido, amidinyl, carboxyl, carboxamide, (C₁-C₆)alkyloxycarbonyl and sulfonyl. More in particular, the term “optionally substituted” refers to radicals further bearing one or more substituents selected from the group of hydroxy; (C₁-C₆)alkyloxy, in particular methoxy and ethoxy; aryl, in particular phenyl; heterocycle, in particular tetrazolyl; halo, in particular chloro and fluoro; trifluoromethyl; amino; amido, in particular mono- and di-((C₁-C₆)alkylcarbonyl)amino, more in particular methylcarbonylamino; and a sulfonyl, in particular (C₁-C₆)alkylsulfonyl, more in particular methylsulfonyl and aminosulfonyl.

In this specification, the term “solvate” refers to a complex of variable stoichiometry formed by a solute (e.g. a compound of Formula (I)) and a solvent. The solvent is a pharmaceutically acceptable solvent as preferably water; such solvent may not interfere with the biological activity of the solute.

In this specification, unless stated otherwise, the term “positive allosteric modulator of mGluR2” or “allosteric modulator of mGluR2” refers also to a pharmaceutically acceptable acid or base addition salt thereof, a stereochemically isomeric form thereof and an N-oxide form thereof.

Pharmaceutical Compositions

Positive allosteric modulators of mGluR2 described herein, and the pharmaceutically acceptable salts, solvates and hydrates thereof can be used in pharmaceutical preparations in combination with a pharmaceutically acceptable carrier or diluent. Suitable pharmaceutically acceptable carriers include inert solid fillers or diluents and sterile aqueous or organic solutions. The positive allosteric modulators of mGluR2 will be present in such pharmaceutical compositions in amounts sufficient to provide the desired dosage amount in the range described herein. Techniques for Formulation and administration of the compounds of the instant invention can be found in Remington: the Science and Practice of Pharmacy, 19^{th} edition, Mack Publishing Co., Easton, Pa. (1995).

The amount of positive allosteric modulators of mGluR2, administered to the subject will depend on the type and severity of the disease or condition and on the characteristics of the subject, such as general health, age, sex, body weight and tolerance to drugs. The skilled artisan will be able to determine appropriate dosages depending on these and other factors. Effective dosages for commonly used CNS drugs are well known to the skilled person. The total daily dose usually ranges from about 0.05-2000 mg.

The present invention relates to pharmaceutical compositions which provide from about 0.01 to 1000 mg of the active ingredient per unit dose. The compositions may be administered by any suitable route. For example orally in the form of capsules, etc. . . . parenterally in the form of solutions for injection, topically in the form of onguents or lotions, ocularly in the form of eye-drops, rectally in the form of suppositories, intranasally or transcutaneously in the form of delivery system like patches.

For oral administration, the positive allosteric modulators of mGluR2 thereof can be combined with a suitable solid or liquid carrier or diluent to form capsules, tablets, pills, powders, syrups, solutions, suspensions and the like.

The tablets, pills, capsules, and the like contain from about 0.01 to about 99 weight percent of the active ingredient and a binder such as gum tragacanth, acacias, corn starch or gelatin; excipients such as dicalcium phosphate; a disintegrating agent such as corn starch, potato starch, alginic acid, a lubricant such as magnesium stearate; and a sweetening agent such as sucrose lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

For parenteral administration the disclosed positive allosteric modulators of mGluR2 can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable salts of the compounds. Dispersions can also be prepared in glycerol, liquid polyethylene glycols and mixtures thereof in oils. Under ordinary conditions of storage and use, these preparations contain a preservative to prevent the growth of microorganisms.

In addition, to the formulations described previously, the compounds may also be formulated as a depot preparation. Such long acting formulations may be administered by implantation, for example, subcutaneously or intramuscularly or by intramuscular injection. Thus, for example, as an emulsion in an acceptable oil, or ion exchange resins, or as sparingly soluble derivatives, for example, as sparingly soluble salts.

Preferably disclosed positive allosteric modulators of mGluR2 or pharmaceutical formulations containing these compounds are in unit dosage form for administration to a mammal. The unit dosage form can be any unit dosage form known in the art including, for example, a capsule, an IV bag, a tablet, or a vial. The quantity of active ingredient in a unit dose of composition is an effective amount and may be varied according to the particular treatment involved. It may be appreciated that it may be necessary to make routine variations to the dosage depending on the age and condition of the patient. The dosage will also depend on the route of administration which may be by a variety of routes including oral, aerosol, rectal, transdermal, subcutaneous, intravenous, intramuscular, intraperitoneal and intranasal.

Pharmacology

The compounds provided in this invention are positive allosteric modulators of metabotropic receptors, in particular they are positive allosteric modulators of mGluR2. The compounds of the present invention do not appear to bind to the glutamate recognition site, the orthosteric ligand site, but instead to an allosteric site within the seven transmembrane region of the receptor. In the presence of glutamate or an agonist of mGluR2, the compounds of this invention increase the mGluR2 response. The compounds provided in this invention are expected to have their effect at mGluR2 by virtue of their ability to increase the response of such receptors to glutamate or mGluR2 agonists, enhancing the response of the receptor. Hence, the present invention relates to a compound for use as a medicine, as well as to the use of a compound according to the invention or a pharmaceutical composition according to the invention for the manufacture of a medicament for treating or preventing a condition in a mammal, including a human, the treatment or prevention of which is affected or facilitated by the neuromodulatory effect of mGluR2 allosteric modulators, in particular positive mGluR2 allosteric modulators.

Also, the present invention relates to the use of a compound according to the invention or a pharmaceutical composition according to the invention for the manufacture of a medicament for treating, or preventing, ameliorating, controlling or reducing the risk of various neurological and psychiatric disorders associated with glutamate dysfunction in a mammal, including a human, the treatment or prevention of which is affected or facilitated by the neuromodulatory effect of mGluR2 positive allosteric modulators.

Where the invention is said to relate to the use of a compound or composition according to the invention for the manufacture of a medicament for e.g. the treatment of a mammal, it is understood that such use is to be interpreted in certain jurisdictions as a method of e.g. treatment of a mammal, comprising administering to a mammal in need of such e.g. a treatment, an effective amount of a compound or composition according to the invention.

In particular, the neurological and psychiatric disorders associated with glutamate dysfunction, include one or more of the following conditions or diseases: acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine (including migraine headache), urinary incontinence, substance tolerance, substance withdrawal (including substances such as opiates, nicotine, tobacco products, alcohol, benzodiazepines, cocaine, sedatives, hypnotics, etc.), psychosis, schizophrenia, anxiety (including generalized anxiety disorder, panic disorder, and obsessive compulsive disorder), mood disorders (including depression, mania, bipolar disorders), trigeminal neuralgia, hearing loss, tinnitus, macular degeneration of the eye, emesis, brain edema, pain (including acute and chronic states, severe pain, intractable pain, neuropathic pain, and post-traumatic pain), tardive dyskinesia, sleep disorders (including narcolepsy), attention deficit/hyperactivity disorder, and conduct disorder.

In particular, the condition or disease is a central nervous system disorder selected from the group of anxiety disorders, psychotic disorders, personality disorders, substance-related disorders, eating disorders, mood disorders, migraine, epilepsy or convulsive disorders, childhood disorders, cognitive disorders, neurodegeneration, neurotoxicity and ischemia.

Preferably, the central nervous system disorder is an anxiety disorder, selected from the group of agoraphobia, generalized anxiety disorder (GAD), obsessive-compulsive disorder (OCD), panic disorder, posttraumatic stress disorder (PTSD), social phobia and other phobias.

Preferably, the central nervous system disorder is a psychotic disorder selected from the group of schizophrenia, delusional disorder, schizoaffective disorder, schizophreniform disorder and substance-induced psychotic disorder.

Preferably, the central nervous system disorder is a personality disorder selected from the group of obsessive-compulsive personality disorder and schizoid, schizotypal disorder.

Preferably, the central nervous system disorder is a substance-related disorder selected from the group of alcohol abuse, alcohol dependence, alcohol withdrawal, alcohol withdrawal delirium, alcohol-induced psychotic disorder, amphetamine dependence, amphetamine withdrawal, cocaine dependence, cocaine withdrawal, nicotine dependence, nicotine withdrawal, opioid dependence and opioid withdrawal.

Preferably, the central nervous system disorder is an eating disorder selected from the group of anorexia nervosa and bulimia nervosa.

Preferably, the central nervous system disorder is a mood disorder selected from the group of bipolar disorders (I & II), cyclothymic disorder, depression, dysthymic disorder, major depressive disorder and substance-induced mood disorder.

Preferably, the central nervous system disorder is migraine.

Preferably, the central nervous system disorder is epilepsy or a convulsive disorder selected from the group of generalized nonconvulsive epilepsy, generalized convulsive epilepsy, petit mal status epilepticus, grand mal status epilepticus, partial epilepsy with or without impairment of consciousness, infantile spasms, epilepsy partialis continua, and other forms of epilepsy.

Preferably, the central nervous system disorder is attention-deficit/hyperactivity disorder.

Preferably, the central nervous system disorder is a cognitive disorder selected from the group of delirium, substance-induced persisting delirium, dementia, dementia due to HIV disease, dementia due to Huntington's disease, dementia due to Parkinson's disease, dementia of the Alzheimer's type, substance-induced persisting dementia and mild cognitive impairment.

Of the disorders mentioned above, the treatment of anxiety, schizophrenia, migraine, depression, and epilepsy are of particular importance.

At present, the fourth edition of the Diagnostic & Statistical Manual of Mental Disorders (DSM-IV) of the American Psychiatric Association provides a diagnostic tool for the identification of the disorders described herein. The person skilled in the art will recognize that alternative nomenclatures, nosologies, and classification systems for neurological and psychiatric disorders described herein exist, and that these evolve with medical and scientific progresses.

Because such positive allosteric modulators of mGluR2, including compounds of Formula I, enhance the response of mGluR2 to glutamate, it is an advantage that the present methods utilize endogenous glutamate.

Because positive allosteric modulators of mGluR2, including compounds of Formula I, enhance the response of mGluR2 to agonists, it is understood that the present invention extends to the treatment of neurological and psychiatric disorders associated with glutamate dysfunction by administering an effective amount of a positive allosteric modulator of mGluR2, including compounds of Formula I, in combination with an mGluR2 agonist.

The compounds of the present invention may be utilized in combination with one or more other drugs in the treatment, prevention, control, amelioration, or reduction of risk of diseases or conditions for which compounds of Formula (I) or the other drugs may have utility, where the combination of the drugs together are safer or more effective than either drug alone.

Methods of Synthesis

The compounds according to the invention, in particular the compounds according to the Formula (I), (II), (II-a), (II-a1), (II-b), (II-b1) and (II-b2) may be prepared by methods known in the art of organic synthesis or by the following synthesis schemes. In all of the schemes described below it is understood that protecting groups for sensitive or reactive groups are employed where necessary in accordance with the general principles of organic chemistry. Protecting groups are manipulated according to standard methods (T. W. Green and P. G. M. Wuts, 1991, Protecting Groups in Organic Synthesis, John Wiley and Sons). These groups are then removed at a convenient stage of the synthesis using methods that are readily apparent to those skilled in the art.

The compounds according to the invention may be represented as a mixture of enantiomers which may be resolved into their individual R- or S-enantiomers. If for instance, a particular enantiomer is required it may be prepared by asymmetric synthesis or by derivation with a chiral auxiliary and the resulting diastereomeric mixture separated. The auxiliary group can then be cleaved to provide the desired pure enantiomers. Alternatively, where the molecule contains a basic functional group such as an amino or an acidic functional group such as a carboxyl functional group, resolution may be performed by fractional crystallization from various solvents as the salt of an optical active acid or by other methods known in the literature (e.g. chiral column chromatography).

Resolution of the final product, an intermediate or a starting material may be performed by any suitable method known in the art (E. L. Eliel, S. H. Wilen and L. N. Mander, 1984, Stereochemistry of Organic Compounds, Wiley-Interscience).

Many of the heterocyclic compounds of Formula (I) to (II-b2) where M₁ or M₂ is heteroaromatic may be prepared using synthetic routes well known in the literature (A. R. Katrizky and C. W. Rees, 1984, Comprehensive Heterocyclic Chemistry, Pergamon Press).

The preparation of mGluR2 positive allosteric modulators disclosed herein is shown in the following synthetic schemes. The synthetic schemes described below are exemplified approaches but should not be taken as the only possible synthetic route to compounds of the present invention. Specific conditions for carrying out these reactions are provided in following examples.

General Synthesis Schemes

In one embodiment of the present invention, compounds of Formula (II-a1), (1-bl) and (II-b2) are exemplified by compound g14 (wherein X is —S—) and may be prepared according to the synthetic sequence illustrated in Scheme 1.

Substituted aryl or heteroaryl compound g1 (wherein W is halide or O-LG, LG is a leaving group selected from tosylate, mesylate) may be converted into a fused ring thiophene 2-carboxylate g3, when treated with thioglycolate in the presence of a base such as Et₃N, K₂CO₃ or the like in a suitable solvent such as DMF or THF at an appropriate temperature (e.g J. Med. Chem, 2001, 44, 988). According to experimental conditions the intermediate compound g2 might be isolated and subsequently treated in alkaline conditions such as Na₂CO₃, t-BuOK, Cs₂CO₃ or the like to afford compound g3.

It is known by a person skilled in the art that substituted aryl or heteroaryl intermediate g1 may be prepared from commercially available aryl or heteroaryl compounds by convenient synthetic methods (e.g. halogenation or metallation) according to well-known procedures widely described in the literature (Tetrahedron, 2001, 57, 4489).

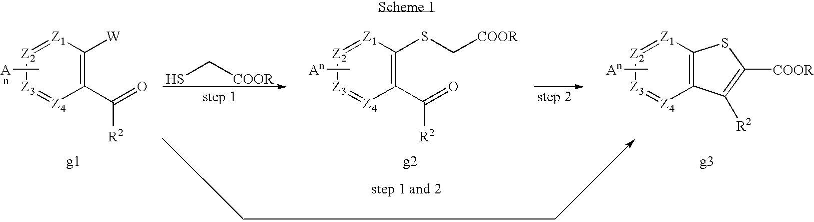

The carboxylate moiety in compound g3 represents an excellent point for introducing suitable —V₁-M₁ groups, wherein M₁ may be, but is not limited to, heterocycles such as benzothiazole, oxadiazole, benzoxazole or isoxazole. The composition of the invention is not limited only to the aforementioned heterocycles but extend to our preferred list of heterocycles which may be introduced through the following schemes (A. R. Katrizky and C. W. Rees, 1984, Comprehensive Heterocyclic Chemistry, Pergamon Press).

As an example, compound g6 may be prepared from compound g5, by converting the hydroxyl group in an convenient leaving group (LG) such as halogen, mesylate or tosylate. Thus formed intermediate may be treated with alcohol M₁-OH in the presence of a base such as K₂CO₃, sodium or NaH, in a appropriate solvent such as alcohols, THF or acetonitrile.

Compound g3 may be transformed into a secondary alcohol g5 using transformations known in the art (Scheme 2).

Alternatively, compound g6 may be directly prepared by reaction of compound g5 with an appropriate M₁-LG group, wherein LG is a leaving group such as halogen, mesylate or tosylate.

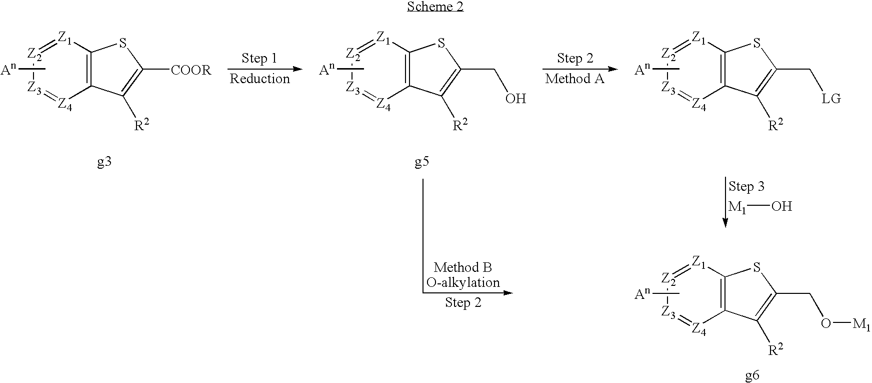

Compound g11 can be prepared according to the synthetic sequence illustrated in Scheme 3. The carboxylate moiety of compound g3 may be converted into a better leaving group (i.e. LG=—N(OMe)Me) then converted to the secondary alcohol g10 using a nucleophilic addition/elimination/reduction sequence. Nucleophilic addition may be performed by using organometallic reagents such as magnesium or lithium derivatives, at a convenient temperature ranging from −78° C. to room temperature in appropriate solvent such as THF. The reduction step may be performed in the presence of hydride reagents such as sodium borohydride in an appropriate solvent such as methanol.

Although these sequences give at first instance a hydroxy-derivative, the hydroxy-derivative may be converted into compound g11 by dehydroxylation of compound g10 using hydride reagents such as R₃SiH or LiAlH₄ promoted by acidic reagents (i.e. Lewis or Brönsted acid) in appropriate solvent such as dichloromethane, diethyl ether or THF.

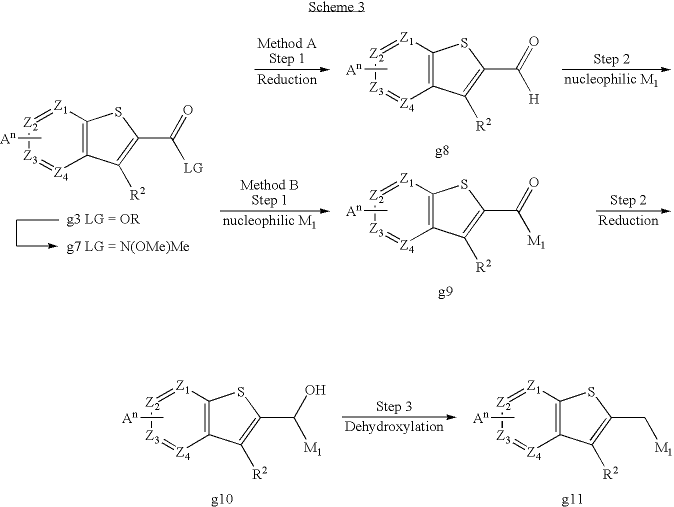

In another embodiment of the present invention, heterocyclic compounds of Formula (II-b1) and (II-b2) exemplified by compound g14 (wherein X is —S—) may be prepared according to the synthetic Scheme 4 from synthesized derivative compound g12.

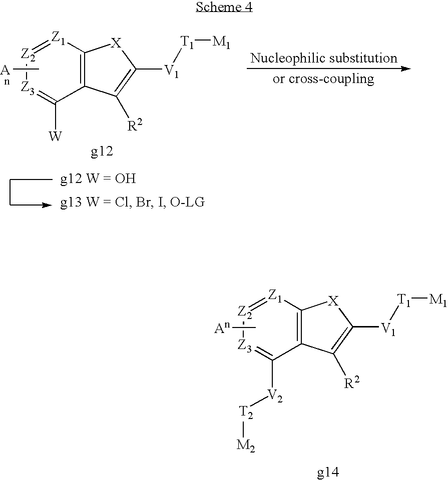

The hydroxyl group in compound g12 can be easily converted into better leaving group (e.g. halides or O-LG; LG is a leaving group selected from tosylate, mesylate) by standard methods known to a person skilled in the art, allowing the introduction of the V₂-T₂-M₂ group through nucleophilic substitution, wherein V₂ is —NR (Scheme 4).

Alternatively, the V₂-T₂-M₂ group may also be introduced by cross-coupling reactions catalyzed by transition metals (e.g. Suzuki, Sonogashira or Heck reactions) wherein V₂ is selected from —(C₁-C₆)alkyl-, —(C₂-C₆)alkenyl- or —(C₂-C₆)-alkynyl-.

In a more specific aspect, compound g14 can be exemplified by compounds of Formula g20 (wherein X═S, Z₁=Z₃═N and Z₂=C). Key compound g18 may be prepared from commercially available or from synthesized 2-aminothiophene 3-carbonitrile (Scheme 8) according to the procedures described in the literature (U.S. Pat. No. 4,196,207).

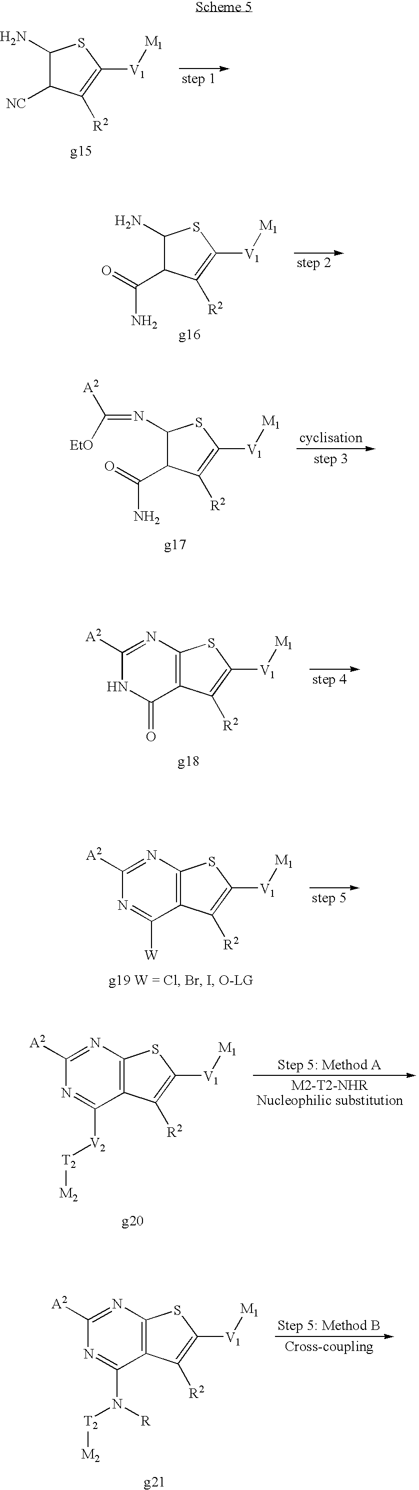

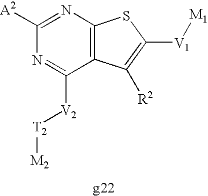

For a person skilled in the art of organic chemistry it is well understood that compounds of the invention, wherein V₂ is selected from —(C₂-C₆)alkenyl- or —(C₂-C₆)-alkynyl-, may be further hydrogenated under catalytic conditions such as Pd/C and H₂ or ammonium formate, to form compound g14 (i.e. g22) wherein V₂ is converted into —(C₂-C₆)alkyl- analogs which are also part of this invention.

In another embodiment of the present invention, the heterocyclic compounds of Formula (I-b) to (II-b2) wherein Z₁ and Z₃ are nitrogen and V₂ is —NH—, exemplified by compound g25 (Scheme 6) may also be prepared according to following synthetic sequence. Suitably substituted heteroaryl g23 may be converted into ethoxymethyleneamino derivative g24 by heating in appropriate orthoester and then treated with appropriate primary amine in a polar and protic solvent such as methanol or ethanol at an appropriate temperature to form compound g25 through a Dimroth's rearrangement (Heterocyclic Chem. 1991, 28, 1709 and Chem. Pharm. Bull. 1997, 45, 832.).

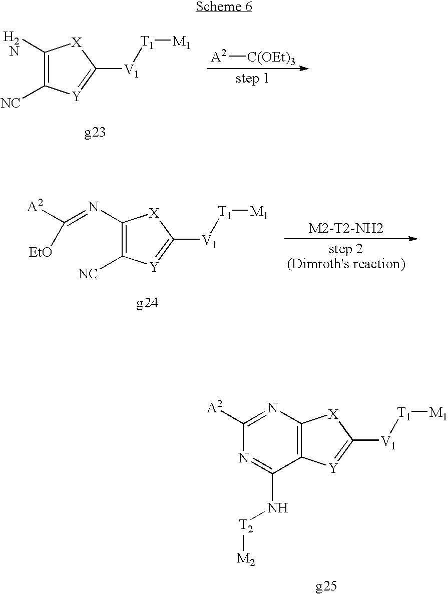

In some cases compound g25 may be prepared by subsequent treatment of the isolated Dimroth intermediate g26 (Scheme 7) with an excess of primary amine or a strong aqueous base such as NaOH, KOH and the like in a polar solvent such as methanol or water at an appropriate temperature.

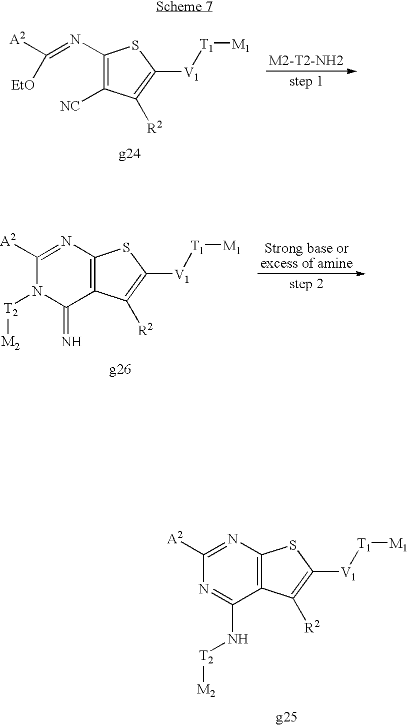

Compounds of Formula (II-b2) exemplified by compound g25 may be prepared from thiophenes g27 bearing an appropriate V₁-M₁ group. Such suitably substituted thiophenes g27 may be prepared from sulfur, malonitrile and appropriate aldehyde or ketone heated in a polar solvent such as DMF, THF and the like in the presence of a base such as triethylamine, at an appropriate temperature (Scheme 8, Journal of Pharmaceutical Sciences, 2001, 90(3), 371; Chem. Ber. 1965, 98, 3571 and Chem. Ber. 1966, 99, 94).

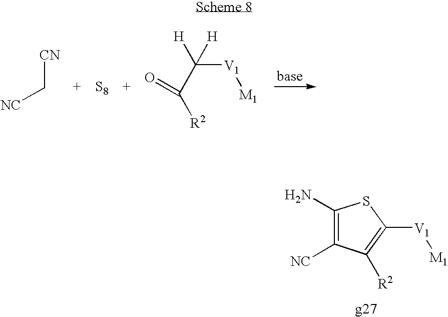

Alternatively, compounds g25 may be prepared by introducing the -T₂-M₂ group by N-alkylation of amino derivatives g28 (Scheme 9).

Compounds of Formula g28 may be prepared by treating appropriate derivative g24 with an alcoholic solution of ammonia.

Alkylation may be performed by displacement of a leaving group W-T₂-M₂ (wherein W is Cl, Br, I or O-LG; where LG is a leaving group selected from tosylate, mesylate) in the presence of a base such as NaH or K₂CO₃ in an appropriate solvent such as DMF, THF or CH₃CN at an appropriate temperature.

Reductive amination may be performed by using suitable aldehydes or ketones (wherein W is ═O) in a presence of a reductive agent such as NaBH₄, NaBH(OAc)₃ and the like. Optionally, an activating lewis acid such as. Ti(OiPr)₄ can be used in an appropriate solvent such as THF at an appropriate pressure and temperature.

Alkylation may also be performed by preparing amide derivatives g29 according to known procedures from carboxylic acid derivatives M2-T2-COOW (wherein W may be H, Cl or LG; LG is any other leaving group) in an appropriate solvent such as CH₂Cl₂, THF or CH₃CN at an appropriate temperature. Homologated derivative g30 can be obtained by a subsequent reduction of the amide function in the presence of reductive agent such as LiAlH₄ in an appropriate solvent such as THF at an appropriate pressure and temperature.

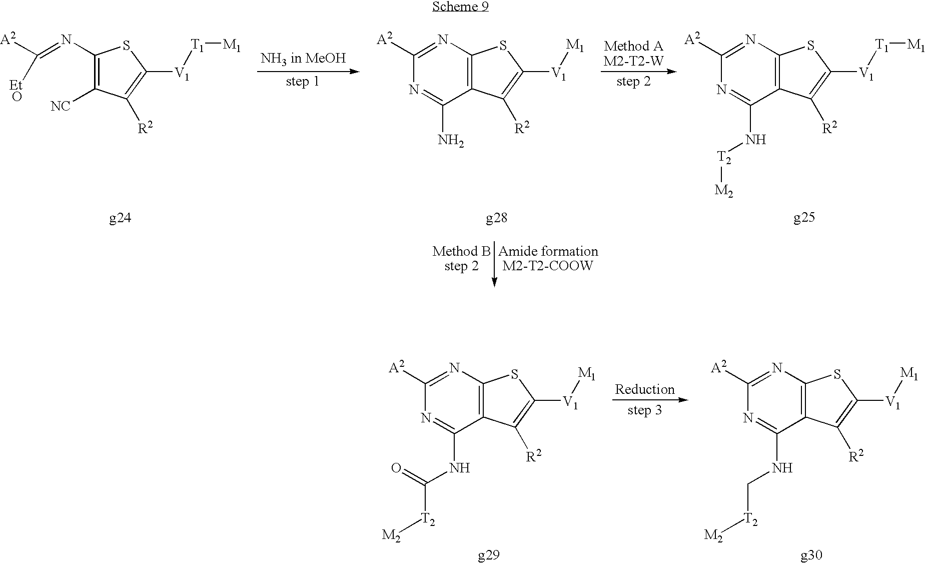

Compounds of Formula (II-b2) exemplified by compound g21 (wherein X═—S—) may be prepared via a similar route as described in Scheme 5 from intermediates g17 (wherein A₂ is an hydroxyl group). A cyclisation step may be performed in mild alkaline condition using a base such as Na₂CO₃ or the like in appropriate solvent and temperature.

The hydroxyl groups in compound g18 may be easily converted into a better leaving group (e.g. halides or O-LG; LG is a leaving group selected from tosylate, mesylate) by standard methods known to a person skilled in the art, allowing the introduction of the V₂-T₂-M₂ group through nucleophilic substitution, (wherein V₂ is —NR, Scheme 10).

Compound g21 may be obtained by introduction of the A₂ group via a nucleophilic substitution of the labile chlorine in a polar solvent such as MeOH, THF, DMF and the like at an appropriate temperature.

Alternatively, the A₂ group may also be introduced by cross-coupling reactions catalyzed by transition metal (e.g. Suzuki, Sonogashira and Heck reactions).

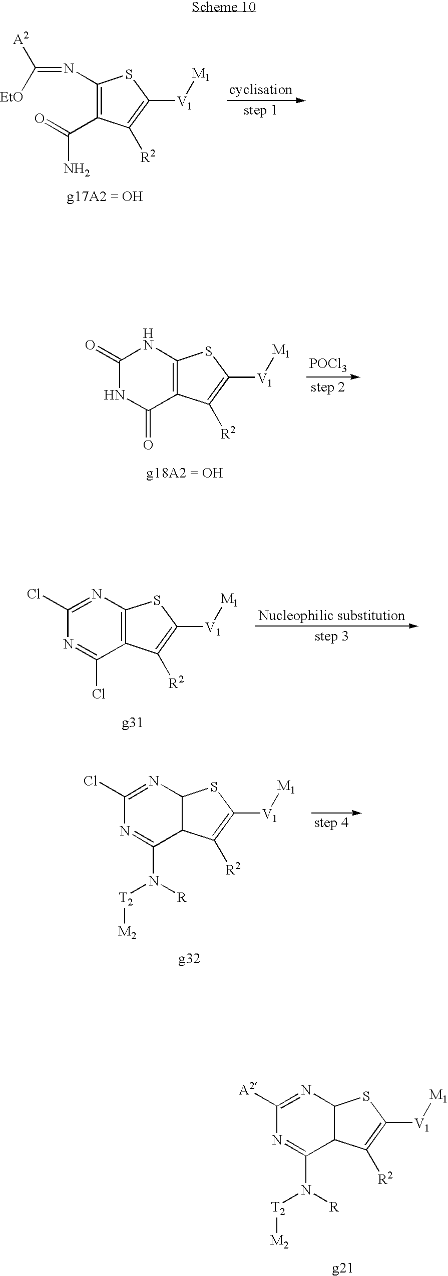

Suitably substituted means in the context of the invention, substituent as preferred in the list of preferred substituents or substituent which may be precursor of the aforementioned preferred substituents and are therefore protected in a manner that a person skilled in the art would recognize (T. W. Green and P. G. M. Wuts, 1991, Protecting Groups in Organic Synthesis, John Wiley et Sons).

In another embodiment of the present invention, the compounds of Formula (II-b2) exemplified by compound g23 (wherein V2=—(CH₂)ₙ—NR—), may be prepared via a similar route as described in previous schemes.

Compound g22 may be hydrolyzed by standard procedure followed by reaction with a primary or secondary amine to lead to compound g25.

One could understand that compounds g22 and g25 represent excellent anchoring point such as acid, nitrile or amide groups for heterocycle formation such as thiazole, oxadiazole, oxazole and isoxazole, affording compound of the invention g23. The composition of the invention is not limited only to the aforementioned heterocycles but extended to our preferred list of heterocycles which can be synthesized through a similar scheme (A. R. Katrizky and C. W. Rees, 1984, Comprehensive Heterocyclic Chemistry, Pergamon Press).

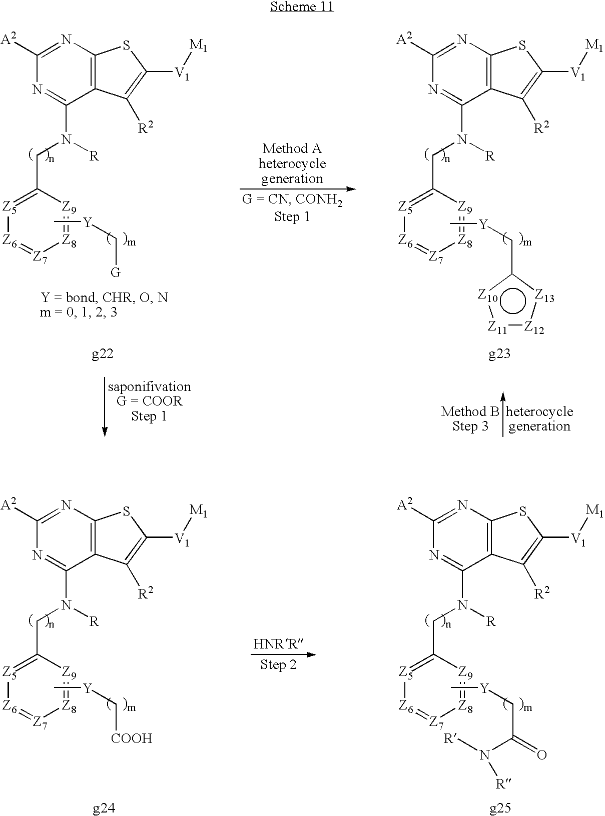

Compounds of Formula (II-b2) exemplified by compound g28 (wherein V2=—(CH₂)ₙ—NR—), may be prepared according to the synthetic Scheme 12. Compound g26 may be prepared according to the aforementioned schemes by introducing an aryl group conveniently substituted by an amino moiety. When necessary the protected amino group in compound g26 may be removed under classical condition well know in the art. The resulting primary amine can be either acylated by standard procedure or submitted to reductive amination as described in the following scheme.

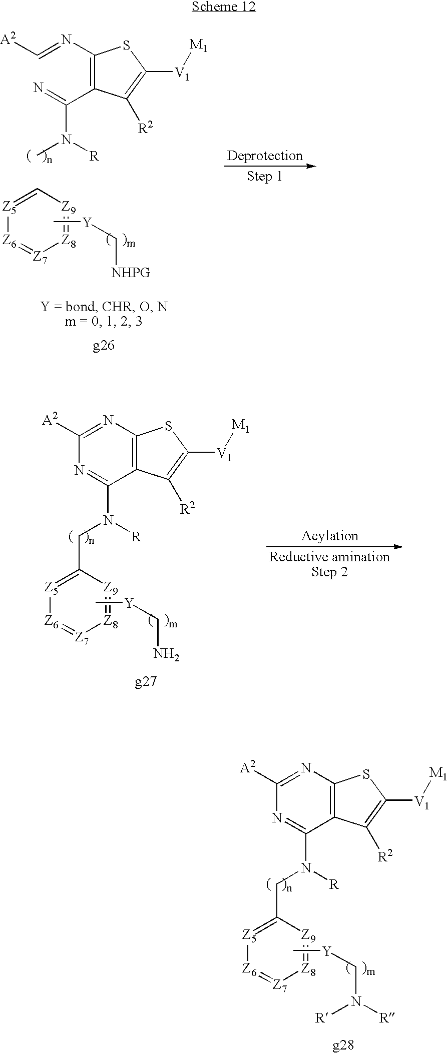

Similarly, compounds of Formula (II-b2) exemplified by compound g31 (wherein V2=10-(CH₂)ₙ—NR—), may be prepared according to the synthetic Scheme 13. Compound g29 may be prepared by introducing an aryl group conveniently substituted by an alkoxy moiety. When necessary the R′ group in compound g29 may be removed under classical condition known by a person skilled in the art. The resulting hydroxyl group can be either acylated or alkylated by standard procedure as described in the following scheme.

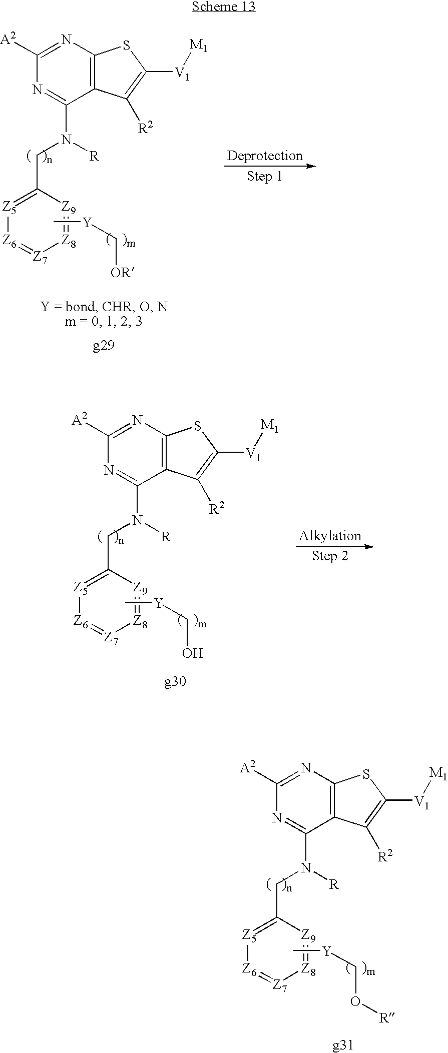

One could understand that compounds g21 can be easily prepared from compounds g25 under classical N-alkylation or N-acylation conditions known by a person skilled in the art (Scheme 14).

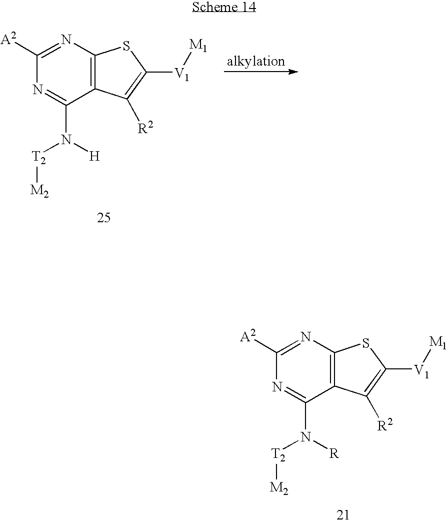

In another embodiment of the present invention, the compounds of Formula (II-b2) exemplified by compound g34 may be prepared from the corresponding amides g33, in the presence of hydride reagents such as LiAlH₄, NaBH₄ and the like, in an appropriate solvent such as THF, methanol and the like, at a convenient temperature.

One could understand that compounds g33 may be easily obtained from carboxylate derivatives g3 using classical saponification/amidation sequence, known by a person skilled in the art (Scheme 15).

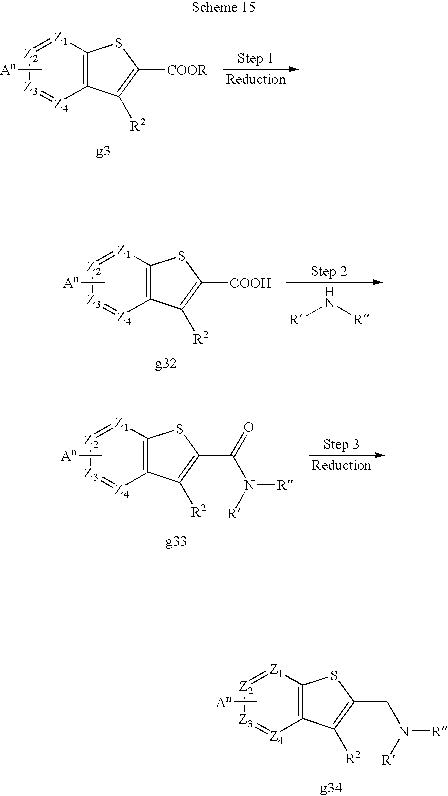

In another embodiment of the present invention, the compounds of Formula (II-b2) may be exemplified by compound g36 by oxidation of a hydroxyl group in classical conditions known by a person skilled in the art. Compound g35 may be prepared according to the aforementioned schemes by introducing M₂-V₂-T₂ group wherein V₂ is bearing a hydroxyl group (Scheme 16).

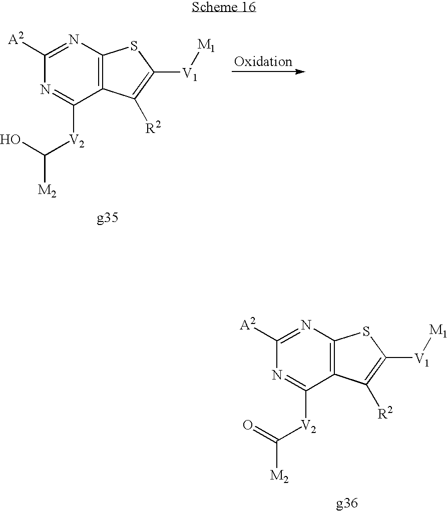

### EXPERIMENTAL

Several methods for preparing the compounds of this invention are illustrated in the following Examples.

Unless otherwise noted, all starting materials were obtained from commercial suppliers and used without further purification.

Specifically, the following abbreviations may be used in the examples and throughout the specification.

| | |
|---|---|
| % (percent) | M (molar) |
| AcOEt (ethyl acetate) | MeOH (methanol) |
| n-BuLi (n-butyllithium) | mg (milligrams) |
| ° C. (Celsius degrees) | MgSO₄ (magnesium sulphate) |
| CDCl₃ (deuterated chloroform) | MHz (megahertz) |
| CHCl₃ (chloroform) | min (minutes) |
| CuI (copper iodide) | μL(microliters) |
| DAST (diethylaminosulfur trifluoride) | mL (milliliters) |
| DCM (dichloromethane) | mmol (millimoles) |
| dec. (decomposition) | Mp or mp (melting point) |
| DIEA (diisopropyl ethyl amine) | N or M (normal or Molar) |
| DMAP (N,N-dimethylaminopyridine) | N₂ (nitrogen) |
| DMF (dimethylformamide) | NaCl (Sodium chloride) |
| DMSO (dimethyl sulfoxide) | NaHCO₃ (sodium hydrogenocarbonate) |
| EDCI.HCl (1-3(Dimethylaminopropyl)-3- | NaOH (sodium hydroxide) |
| ethylcarbodiimide, hydrochloride) | |
| Et₂O (diethyl ether) | Na₂SO₄ (sodium sulphate) |
| g (grams) | NH₄Cl (ammonium chloride) |
| h (hour) | NH₄OH (ammonium hydroxide) |
| ¹H (proton) | NMR (Nuclear Magnetic Reasonance) |
| HCl (hydrochloric acid) | PdCl₂(PPh₃)₂ (Bis(triphenylphosphine) |
| | palladium (II) dichloride |
| HOBT (1-hydroxybenzotriazole) | Pd(PPh₃)₄ |
| | (tetrakis(triphenylphosphine)palladium(0) |
| HPLC (High Pressure Liquid Chromatography) | P₂O₅ (phosphorus pentoxide) |
| H₂SO₄ (Sulfuric acid) | POCl₃ (phosphorus oxychloride) |
| Hz (Hertz) | R.T. or RT (Room Temperature) |
| K₂CO₃ (potassium carbonate) | Tf₂O triflic anhydride |
| KI (potassium iodide) | THF (tetrahydrofuran) |
| LCMS (Liquid Chromatography Mass Spectrum) | TLC (tin chromatography layer) |
| LiAlH₄ (lithium aluminium hydride) | Rt (retention time) |

All references to brine refer to a saturated aqueous solution of NaCl. Unless otherwise indicated, all temperatures are expressed in ° C. (degrees Centigrade). All reactions are conducted not under an inert atmosphere at room temperature unless otherwise noted.

The microwave oven used is an apparatus from Biotage (Optimizer™) equipped with an internal probe that monitors reaction temperature and pressure, and maintains the desired temperature by computer control.

### EXAMPLES

### Example 1

### 6-ethyl-N-(1-phenylethyl)thieno[2,3-d]pyrimidin-4-amine (Final Compound 74)

### a) 5-ethyl-2-ethoxymethyleneamino-3-cyanothiophene

According to Scheme 6 Step 1: Title compound was prepared according to procedure described in the literature (U.S. Pat. No. 04,196,207) from 2-amino-3-cyano-5-ethylthiophene (5.91 mmol) and triethylorthoformate (59.13 mmol). The crude material (1.151 g) was used directly in the next step.

### b) 6-ethylthieno[2,3-d]pyrimidin-4-amine

According to Scheme 9 Step 1: To 5-ethyl-2-ethoxymethyleneamino-3-cyanothiophene (4.08 mmol) was added a 7N solution of ammonia in methanol (10 ml). The mixture was stirred at r.t. for 15 hours. The solution was concentrated till dryness, yielding 0.700 g of crude material. The residue was taken up in acetonitrile and filtered off and dried, yielding title compound (0.513 g, 70%).

The mother layer was evaporated till dryness (m=0.187 g) and purified by flash chromatography over silica gel (Flashpack 5 g SiO2 (20-40 um); AcOEt/Methanol 95:5) yielding additional amount of title compound (0.080 g, 11%).

### c) 6-ethyl-N-(1-phenylethyl)thieno[2,3-d]pyrimidin-4-amine

According to Scheme 9 Method A Step 2: To a solution of 6-ethylthieno[2,3-d]pyrimidin-4-amine (0.56 mmol) in dimethylformamide (10 ml) was added portionwise sodium hydride (55% in mineral oil, 0.61 mmol). The reaction mixture was stirred for 15 minutes and alpha-methylbenzyl bromide (0.84 mmol) was then added. The mixture was stirred at r.t. for 2 hours then poured onto water and extracted with ethyl acetate. The organic layer was washed with water, dried over MgSO4, filtered and evaporated till dryness. The residue (0.192 g) was purified by flash chromatography over silica gel (Flashpack 10 g SiO2 (40-60 um); dichloromethane/AcOEt 90:10) yielding title compound (0.089 g, 56%) as a white solid;

mp: 141° C.; LC (XTerra RP₁₈, 3.5 μm, 3.0×50 mm Column): Rt=4.48 min; MS m/z (CI) [MH]⁺=284; ¹H NMR (500 MHz, CDCl₃) δ 1.36 (3H, t, 7.52 Hz), 1.66 (3H, d, 6.85 Hz), 2.91 (2H, qd, 7.52 Hz, 1.08 Hz), 5.26 (1H, m), 5.6 (1H, qd, 6.85 Hz, 7.09 Hz), 6.8 (1H, d, 1.08 Hz), 7.29 (1H, m), 7.37 (2H, m), 7.42 (2H, m), 8.44 (1H, s).

### Example 2

### N-phenethyl-6-propylthieno[2,3-d]pyrimidin-4-amine (Final Compound 79)

### a) 5-propyl-2-ethoxymethyleneamino-3-cyanothiophene

According to Scheme 6 Step 1: Title compound was prepared according to procedure described in the literature (U.S. Pat. No. 04,196,207) from 2-amino-3-cyano-5-propylthiophene (0.50 g, 3.00 mmol) and triethylorthoformate (30.00 mmol). The crude material (0.710 g) was used directly in the next step.

### b) N-phenethyl-6-propylthieno[2,3-d]pyrimidin-4-amine

According to Scheme 7: A mixture of 5-propyl-2-ethoxymethyleneamino-3-cyanothiophene (0.48 mmol) and phenethylamine (2.25 mmol) in methanol (1 ml) was heated at 150° C. under microwave for 1 hour. The solvent was removed in vacuo and the residue was taken up a 1N solution of sodium hydroxide (3 ml) and then heated at 150° C. under microwave for 30 minutes.

Water was added and the reaction mixture was extracted with ethyl acetate. The organic layer was dried over MgSO4, filtered, and evaporated till dryness. The residue (0.461 g) was purified by chromatography over silica gel (Flashmart Pack: 25 g/60-40 um, eluent cyclohexane/ethyl acetate/1:1) and crystallized in pentane, yielding title compound (0.091 g, 68%) as yellow crystals;

mp: 110.5° C.; LC (XTerra RP₁₈, 3.5 μm, 3.0×50 mm Column): Rt=4.71 min; MS m/z (CI) [MH]⁺=298; ¹H NMR (500 MHz, CDCl₃) δ 1.01 (3H, t, 7.34 Hz), 1.75 (2H, tq, 7.34 Hz, 8.01 Hz), 2.83 (2H, t, 8.01 Hz), 3.01 (2H, t, 6.87 Hz), 3.89 (2H, td, 6.87 Hz, 5.92 Hz), 5.04 (1H, s), 6.66 (1H, s), 7.26 (3H, m), 7.34 (2H, m), 8.48 (1H, s).

### Example 3

### N-(4-methoxyphenethyl)-N,2,6-trimethylthieno[2,3-d]pyrimidin-4-amine (Final Compound 51)

### a) 5-methyl-2-ethoxyethyleneamino-3-cyanothiophene

According to Scheme 6 Step 1: Title compound was prepared according to procedure described in the literature (U.S. Pat. No. 04,196,207) from 2-amino-3-cyano-5-methylthiophene (2.76 g, 20.0 mmol) and triethylorthoacetate (32.0 g, 0.20 mol). The crude material (3.87 g) was used directly in the next step.

### b) N-(4-methoxyphenethyl)-2,6-dimethylthieno[2,3-d]pyrimidin-4-amine

According to Scheme 7: Title compound was prepared according to Example 2—step b, from 5-methyl-2-ethoxyethyleneamino-3-cyanothiophene (1.00 g, 4.80 mmol) and 4-methoxy-phenethylamine (3.51 ml, 24.01 mmol), then purified by chromatography over silica gel (Flashmart Pack: 25 g/60-40 um, eluent dichloromethane/methanol/NH₄OH 95:5:0.1) and crystallized in diisopropylether, yielding title compound (0.241 g, 16%) as pale yellow crystals.

### c) N-(4-methoxyphenethyl)-N,2,6-trimethylthieno[2,3-d]pyrimidin-4-amine

According to Scheme 14: To a solution of N-(4-methoxyphenethyl)-2,6-dimethylthieno[2,3-d]pyrimidin-4-amine (0.050 g, 0.160 mmol) in THF (30 ml) at 0° C. under nitrogen atmosphere was added sodium hydride (0.012 g, 0.480 mmol) portionwise. The mixture was stirred for 45 minutes at 0° C., then iodomethane (0.07 g, 0.480 mmol) was added dropwise at 0° C. The mixture was allowed to warm at r.t. for 3 hours. To complete the reaction, a new excess of sodium hydride (0.024 g, 0.960 mmol) and iodomethane (0.14 g, 0.960 mmol) was added and the reaction mixture was stirred at r.t. overnight.

The reaction mixture was poured onto ice-water and extracted with ethyl acetate. The organic layer was dried over MgSO4, filtered and evaporated till dryness. The residue (1.0 g) was purified by chromatography over silica gel (Flashmart Pack: 25 g/60-40 um, eluent cyclohexane/ethyl acetate/2:1) then crystallized in pentane yielding title compound (0.028 g, 53%), as white crystals;

mp: 81° C.; LC (XTerra RP₁₈, 3.5 μm, 3.0×50 mm Column): Rt=3.63 min; MS m/z (CI) [MH]⁺=328; ¹H NMR (500 MHz, CDCl₃) δ 2.52 (3H, d, 1.04 Hz), 2.58 (3H, s), 2.94 (2H, t, 7.61 Hz), 3.28 (3H, s), 3.81 (3H, s), 3.92 (2H, t, 7.61 Hz), 6.86 (2H, d, 8.57 Hz), 7 (1H, d, 1.04 Hz), 7.17 (2H, d, 8.57 Hz).

### Example 4

### N,N-dimethyl-4-(phenethylamino)thieno[2,3-d]pyrimidine-6-carboxamide (Final Compound 62)

### a) ethyl 2-(6-chloro-5-formylpyrimidin-4-ylthio)acetate

According to Scheme 1 Step 1: To a mixture of 2,4-dichloropyrimidine-3-carboxaldehyde (3.14 g, 17.8 mmol) and diethylisopropylamine (2.30 g, 17.8 mmol) in dichloromethane (60 mL) at −10° C. under nitrogen atmosphere was added over 30 min a solution of methylthioglycolate (1.92 g, 16.0 mmol) in dichloromethane (30 mL). The reaction mixture was allowed to warm to room temperature for 2 hours, then poured onto water. The organic layer was washed with brine, dried over MgSO4, filtered and concentrated in vacuum, yielding title compound (5.0 g).

### b) ethyl 4-chlorothieno[2,3-d]pyrimidine-6-carboxylate

According to Scheme 1 Step 2: A mixture of ethyl 2-(6-chloro-5-formylpyrimidin-4-ylthio)acetate (4.63 g, 17.8 mmol) and diethylisopropylamine (2.30 g, 17.8 mmol) in cyclohexanol under inert atmosphere was heated at 120° C. for 90 min. The solvent was removed and the residue was purified by chromatography over silica gel (Flashmart Pack: 25 g/60-40 um, eluent dichloromethane/cyclohexane 1:1), yielding title compound (2.50 g, 58%), as a light yellow solid.

### c) ethyl 4-(phenethylamino)thieno[2,3-d]pyrimidine-6-carboxylate

According to Scheme 5 Method A Step 5: A mixture of ethyl 4-chlorothieno[2,3-d]pyrimidine-6-carboxylate (2.5 g, 10.3 mmol), potassium carbonate (2.14 g, 15.5 mmol) and phenethylamine (1.55 mL, 12.4 mmol) in acetonitrile (20 mL) was heated at 50° C. for 2 hours. The reaction mixture was filtered then the organic layer was washed with water and brine, dried over MgSO4, filtrated and evaporated till dryness, yielding the title compound (3.11 g, 92%) as a white solid used directly in the next step.

### d) 4-(phenethylamino)thieno[2,3-d]pyrimidine-6-carboxylic acid

According to Scheme 15 Step 1: A solution of ethyl 4-(phenethylamino)thieno[2,3-d]pyrimidine-6-carboxylate (1.50 g, 4.6 mmol) and lithium hydroxide (2.10 g, 27.0 mmol) in a 1:1 mixture of THF/water (1000 ml) was stirred at r.t. overnight. The mixture was made slight acidic (pH3-4) with a 1N solution of HCl and the precipitate was filtered, washed with water and dried over night at 40° C. under vacuum, yielding title compound (0.95 g, 70%) as a white powder.

### e) N,N-dimethyl-4-(phenethylamino)thieno[2,3-d]pyrimidine-6-carboxamide

According to Scheme 15 Step 2: To a solution of 4-(phenethylamino)thieno[2,3-d]pyrimidine-6-carboxylic acid (0.10 g, 0.33 mmol) in dichloromethane (3 mL) was added hydroxybenzotriazole hydrate (0.055 g, 0.44 mmol) and 1-(3-dimethylaminopropyl)-3-ethylcarbodiimide hydrochloride (0.10 g, 0.50 mmol). After 10 minutes dimethylamine (0.2 ml of a 2M solution, 0.44 mmol) was slowly added and the reaction mixture was stirred at r.t. overnight. Water was added and the solution was extracted twice with dichloromethane. The organic layer was washed with sodium carbonate and brine, dried over MgSO₄, filtered and evaporated till dryness. The residue was purified by chromatography over silica gel (Flashmart Pack: 25 g/60-40 um, eluent dichloromethane/methanol 99:1) yielding title compound (0.004 g, 4%), a yellow solid;

mp: 157° C.; LC (XTerra RP₁₈, 3.5 μm, 3.0×50 mm Column): Rt=3.63 min; MS m/z (CI) [MH]⁺=327; ¹H NMR (300 MHz, CDCl₃) δ 1.18 (6H, s), 2.95 (2H, t), 3.85 (2H, td), 5.71 (1H, m), 7.17-7.29 (5H, m), 7.37 (1H, s), 8.46 (1H, s).

### Example 5

### N-(3-(4-methoxyphenyl)propyl)-2,6-dimethylthieno[2,3-d]pyrimidin-4-amine (Final Compound 56)

### a) 2,6-dimethylthieno[2,3-d]pyrimidin-4-amine

According to Scheme 9 Step 1: Title compound was prepared according to EXAMPLE 1—step b, from 2-ethoxyethylene-5-methyl-3-cyanothiophene (1.00 g, 4.801 mmol) yielding title compound as brown crystals (0.550 g, 64%).

### b) 3-(4-methoxyphenyl)-N-(2,6-dimethylthieno[2,3-d]pyrimidin-4-yl)propanamide

According to Scheme 9 Method B Step 2: To a solution of 3-(4-methoxyphenyl)propionic acid (0.202 g, 1.12 mmol) and 1-(3-dimethylaminopropyl)-3-ethylcarbodiimide hydrochloride (0.32 g, 1.7 mmol) in dichloromethane (7 ml) were added hydroxybenzotriazole hydrate (0.19 g, 1.2 mmol) then triethylamine (0.32 ml, 2.23 mmol). 2,6-dimethylthieno[2,3-d]pyrimidin-4-amine (0.20 g, 1.12 mmol) was finally added and the reaction mixture was stirred at 50° C. for 17 hours. Water was then added and the reaction mixture was extracted with ethyl acetate. The organic layer was dried over MgSO4, filtered, and evaporated till dryness. The crude material (0.150 g) was purified by chromatography over silica gel (Flashmart Pack: 25 g/60-40 um, eluent: dichloromethane/ethyl acetate 80:20), washed with pentane and crystallized in acetonitrile, yielding title compound as white solid (0.039 g, 10%).

### d) N-(3-(4-methoxyphenyl)propyl)-2,6-dimethylthieno[2,3-d]pyrimidin-4-amine

According to Scheme 9 Method B Step 3: To a solution of 3-(4-methoxyphenyl)-N-(2,6-dimethylthieno[2,3-d]pyrimidin-4-yl) propanamide (0.29 mmol) in THF (5 ml) at 0° C. was added portionwise lithium aluminium hydride (0.44 mmol). The reaction mixture was stirred at 0° C. for 1 hour then at r.t. for 24 hours. When the reaction is not completed, a slight excess of lithium aluminium hydride can be added and the reaction mixture heated at 50° C. for a couple of hours.

The reaction mixture was carefully poured onto ice-water, then filtered over celite and washed with ethyl acetate. The organic layer was dried over MgSO₄, filtered and evaporated till dryness. The residue was purified by flash chromatography over silica gel (Flashsmart Pack: 5 g/60-40 um; eluent dichloromethane/ethyl acetate 4:1) then crystallized in pentane, yielding title compound (0.017 g, 17%) as a white solid;

mp: 120° C.; LC (XTerra RP₁₈, 3.5 μm, 3.0×50 mm Column): Rt=3.23 min; MS m/z (CI) [MH]⁺=328; ¹H NMR (300 MHz, CDCl₃) δ 1.99 (2H, tt, 7.17 Hz, 7.43 Hz), 2.51 (3H, d, 1.02 Hz), 2.57 (3H, s), 2.71 (2H, t, 7.43 Hz), 3.64 (2H, td, 5.63 Hz, 7.17 Hz), 3.8 (3H, s), 4.75 (1H, s), 6.5 (1H, d, 1.02 Hz), 6.84 (2H, d, 8.71 Hz), 7.14 (2H, d, 8.71 Hz)

### Example 6

### 2-ethyl-N-phenethylthieno[2,3-b]pyridin-4-amine (Final Compound 96)

### a) ethyl 4-iodothieno[2,3-b]pyridine-2-carboxylate

According to Scheme 1 Step 1 and 2: A mixture of 2-chloro-3-formyl-4-iodopyridine (1.00 g, 3.74 mmol) and potassium carbonate (0.568 g, 4.11 mmol) in DMF (8 ml) was heated at 80° C. Then ethyl-2-mercaptoacetate (0.396 ml, 3.59 mmol) was added dropwise at 80° C. for 2 hours. Then, the mixture was heated at that temperature for 19 hours, poured onto water (200 ml) and extracted with ethyl acetate. The organic layer was dried over MgSO4, filtered, and evaporated till dryness. The residue (1.25 g), was purified by chromatography (C18, Flashmart Pack: 65 g/60-40 um, eluent ACN/water 60:40) yielding title compound (0.415 g, 46%) as white solid.

### b) ethyl 4-(phenethylamino)thieno[2,3-b]pyridine-2-carboxylate

According to Scheme 5 Method A Step 5: A mixture of ethyl 4-iodothieno[2,3-b]pyridine-2-carboxylate (0.415 g, 1.72 mmol), phenethylamine (0.323 ml, 2.58 mmol) and triethylamine (0.478 ml, 3.43 mmol) in acetonitrile (3 ml) was heated at 180° C. under micro wave for 1 hour. Water was added and the reaction mixture was extracted with ethyl acetate. The organic layer was dried over MgSO4, filtered, and evaporated till dryness. The residue (0.81 g) was purified by chromatography over silica gel (Flashmart Pack: 50 g/60-40 um, eluent DCM/ethyl acetate 90:10), then crystallized in pentane yielding title compound (0.270 g, 48%) as a yellow solid.

### c) 1-(4-(phenethylamino)thieno[2,3-b]pyridin-2-yl)ethanone

According to Scheme 3 Method B Step 1: To a solution of ethyl 4-(phenethylamino)thieno[2,3-b]pyridine-2-carboxylate (0.320 g, 0.98 mmol) in THF (10 ml) at −78° C. and under nitrogen atmosphere was added dropwise a 1.6M solution of methyl lithium (1.8 ml, 2.9 mmol) over 20 min. The mixture was stirred at −78° C. for 3 hours then a little of water was slowly added and the mixture was allowed to warm at r.t.

The reaction mixture was extracted with ethyl acetate and the organic layer was dried over MgSO4, filtered and evaporated till dryness. The residue (0.473 g) was purified by chromatography over silica gel (Flashmart Pack: 25 g/60-40 um, eluent cyclohexane/ethyl acetate 1:1) yielding title compound (0.062 g, 21%) as a yellow solid.

### d) 1-(4-(phenethylamino)thieno[2,3-b]pyridin-2-yl)ethanol

According to Scheme 3 Method B Step 2: To a solution of 1-(4-(phenethylamino)thieno[2,3-b]pyridin-2-yl)ethanone (0.062 g, 0.21 mmol) in methanol (6 ml) at 0° C., sodium borohydride (0.026 g, 0.69 mmol) was added portionwise. The mixture was stirred at 0° C. for 1 h30 min, then water was slowly added and the reaction mixture was extracted with ethyl acetate. The organic layer was washed with water, dried over MgSO4, filtered, and evaporated till dryness, yielding title compound as a yellow solid (0.060 g, 96%).

### e) 2-ethyl-N-phenethylthieno[2,3-b]pyridin-4-amine

According to Scheme 3 Step 3: To a solution of 1-(4-(phenethylamino)thieno[2,3-b]pyridin-2-yl)ethanol (0.062 g, 0.21 mmol) in diethyl ether (6 ml) was added at r.t. aluminum chloride (0.14 g, 1.00 mmol) portionwise. The mixture was cooled at 0° C. and lithium aluminum hydride (0.039 g, 1.00 mmol) was carefully added and the reaction mixture was stirred at 0° C. for 2 hours. Ethyl acetate was slowly added to destroy the excess of hydride and water was slowly added. The reaction mixture was extracted with ethyl acetate. The organic layer was washed with water and then dried over MgSO4, filtered and evaporated till dryness. The residue (0.030 g) was purified by flash chromatography over silica gel (Flashsmart Pack: 10 g/60-40 um; eluent dichloromethane/ethyl acetate 9:1) then crystallized in pentane, yielding title compound (0.011 g, 19%) as a solid;

mp: 88° C.; LC (XTerra RP₁₈, 3.5 μm, 3.0×50 mm Column): Rt=3.19 min; MS m/z (CI) [MH]⁺=283; ¹H NMR (300 MHz, CDCl₃) δ 1.35 (3H, td, 7.43 Hz, 1.02 Hz), 2.89 (2H, q, 7.43 Hz), 3.01 (2H, t, 7.04 Hz), 3.56 (2H, td, 5.89 Hz, 7.04 Hz), 4.51 (1H, s), 6.42 (1H, d, 5.38 Hz), 6.68 (1H, d, 1.02 Hz), 7.22-7.37 (5H, m), 8.2 (1H, d, 5.38 Hz).

### Example 7

### 2-methoxy-6-methyl-N-phenethylthieno[2,3-d]pyrimidin-4-amine (Final Compound 15)

### a) 6-methylthieno[2,3-d]pyrimidine-2, 4(1H, 3H)-dione

According to Scheme 10 Step 1: A mixture of 2-ethoxyethyleneamino-5-methylthiophene-3-carboxamide (1.45 g, 6.35 mmol) and sodium carbonate (4.02 g, 38.2 mmol) in water (15 ml) was heated at 150° C. under microwave for 10 minutes. The mixture was poured onto water and neutralized at pH=7 with concentrated hydrochloric acid, filtered and dried, yielding title compound as a brown solid (0.890 g, 77%).

### b) 2,4-dichloro-6-methylthieno[2,3-d]pyrimidine

According to Scheme 10 Step 2: 6-methylthieno[2,3-d]pyrimidine-2,4(1H,3H)-dione (0.890 g, 0.488 mmol) was added by portion into phosphorous oxychloride (5.92 ml, 63.5 mmol) for 20 min. The mixture was stirred at r.t for 10 minutes, then pyridine (9.77 mmol) was added dropwise for 5 min. The mixture was then heated at 110° C. for 45 min. The excess of phosphorous oxychloride was removed in vacuo and the residue was taken up in dichloromethane and quickly washed with cold water. The organic phase was dried over MgSO4, filtered, and evaporated till dryness, yielding crude title compound as a brown solid, (0.790 g, 74%).

### c) 2-chloro-6-methyl-N-phenethylthieno[2,3-d]pyrimidin-4-amine

According to Scheme 10 Step 3: A suspension of 2,4-dichloro-6-methylthieno[2,3-d]pyrimidine (0.700 g, 3.20 mmol), phenethylamine (0.481 ml, 3.83 mmol) and potassium carbonate (0.662 g, 4.79 mmol) in acetonitrile (6 ml) was heated at 80° C. to 17 hours. Then, a little of water was added to the mixture was extracted with ethyl acetate. The organic layer was washed with water, dried over MgSO4, filtered, and evaporated till dryness. The residue (brown solid, 1.00 g) was purified by chromatography over silica gel (Flashmart Pack: 50 g/60-40 um, eluent DCM) yielding title compound (0.900 g, 92.7%) as a yellow solid.

### d) 2-methoxy-6-methyl-N-phenethylthieno[2,3-d]pyrimidin-4-amine

According to Scheme 10 Step 4: To solution of sodium methoxide (0.35 mmol from 0.008 g of sodium) in methanol at r.t. was added 2-chloro-6-methyl-N-phenethylthieno[2,3-d]pyrimidin-4-amine (0.070 g, 0.23 mmol). The mixture was heated at 135° C. under microwave for 1 hour. The cold reaction mixture was added water and extracted with ethyl acetate. The organic layer was dried over MgSO4, filtered, and evaporated till dryness. The residue was purified by flash chromatography over silica gel (Flashsmart Pack: 10 g/60-40 um; eluent dichloromethane), yielding title compound (0.051 g, 74%) as a white solid;

mp: 138.5° C.; LC (XTerra RP₁₈, 3.5 μm, 3.0×50 mm Column): Rt=4.45 min; MS m/z (CI) [MH]⁺=300; ¹H NMR (300 MHz, CDCl₃) δ 2.4 (3H, d, 1.02 Hz), 2.9 (2H, t, 6.66 Hz), 3.78 (2H, td, 5.89 Hz, 6.66 Hz), 3.93 (3H, s), 4.91 (1H, s), 6.45 (1H, d, 1.02 Hz), 7.17 (3H, m), 7.26 (2H, m).

### Example 8

### N-(3-hydroxyphenethyl)-2,6-dimethylthieno[2,3-d]pyrimidin-4-amine (Final Compound 22)

### a) 2-amino-5-methylthiophene-3-carboxamide

According to Scheme 5 Step 1: 2-amino-5-methylthiophene-3-carbonitrile (4.00 g, 28.9 mmol) in concentrated sulfuric acid (38.8 ml) was stirred at r.t. for 20 hours. The mixture was poured onto ice-water (250 g) and neutralized to pH=7 with a concentrated sodium hydroxide solution. The mixture was extracted with ethyl acetate and the organic layer was dried over MgSO4, filtered and evaporated till dryness. The residue was purified by chromatography over silica gel (Flashmart Pack: 85 g/60-40 um, eluent: ethyl acetate) yielding title compound (3.10 g, 69%) as a brown solid.

### b) 2,6-dimethylthieno[2,3-d]pyrimidin-4(3H)-one

According to Scheme 5 Step 2 and 3: A solution of 2-amino-5-methylthiophene-3-carboxamide (2.00 g, 12.8 mmol) and triethylorthoacetate (7 ml, 38.4 mmol) in toluene (10 ml) was heated 170° C. under micro wave for 1 hour, three times. The solvent was removed in vacuo and the residue was taken up in dichloromethane, filtered and dried, yielding title compound (1.56 g, 67%) as a brown solid.

### c) 4-chloro-2,6-dimethylthieno[2,3-d]pyrimidine

According to Scheme 5 Step 4: A mixture of 2,6-dimethylthieno[2,3-d]pyrimidin-4(3H)-one (1.55 g, 8.660 mmol) in phosphorous oxychloride (10 ml, 107.5 mmol) was heated at 100° C. for 2 hours. The mixture was evaporated till dryness and the residue (brown oil, 3.00 g) was purified by chromatography over silica gel (Flashmart Pack: 70 g/60-40 um, eluent: dichloromethane/ethyl acetate/50:50, then ethyl acetate) yielding title compound (1.70 g, 100%) as a yellow solid.

### d) N-(3-hydroxyphenethyl)-2,6-dimethylthieno[2,3-d]pyrimidin-4-amine

According to Scheme 5 Method A Step 5: Title compound was prepared according to EXAMPLE 7—step c, from 2,6-dimethyl-4-chlorothieno[2,3-d]pyrimidine (0.35 mmol) and 3-hydroxyphenethylamine hydrochloride (0.53 mmol), then purified by flash chromatography over silica gel (Flashmart Pack: 10 g/60-40 um, eluent cyclohexane/ethyl acetate 1:1), yielding title compound (0.040 g, 38%) as white solid;

mp: 162.5° C.; LC (XTerra RP₁₈, 3.5 μm, 3.0×50 mm Column): Rt=2.60 min; MS m/z (CI) [MH]⁺=300; ¹H NMR (300 MHz, CDCl₃) δ 2.51 (3H, s), 2.59 (3H, s), 2.93 (2H, t, 6.61 Hz), 3.85 (2H, td, 6.01 Hz, 6.61 Hz), 6.64 (1H, s), 6.73-6.81 (3H, m), 7.19 (1H, dd, 7.8 Hz, 8.4 Hz).

### Example 9

### 2-(2,6-dimethylthieno[2,3-d]pyrimidin-4-ylamino)-1-phenylethanone (Final Compound 12)

### a) 2-(2,6-dimethylthieno[2,3-d]pyrimidin-4-ylamino)-1-phenylethanol

According to Scheme 5 Method A Step 5: Title compound was prepared according to EXAMPLE 8—step d, from 2,6-dimethyl-4-chlorothieno[2,3-d]pyrimidine (0.100 g, 0.50 mmol) and 2-amino-1-phenylethanol (0.083 g, 060 mmol), then purified by flash chromatography over silica gel (Flashmart Pack: 10 g/60-40 um, eluent cyclohexane/ethyl acetate 3:2), yielding title compound (0.047 g, 31%) as an orange solid.

### b) 2-(2,6-dimethylthieno[2,3-d]pyrimidin-4-ylamino)-1-phenylethanone

According to Scheme 16: To a solution of 2-(2,6-dimethylthieno[2,3-d]pyrimidin-4-ylamino)-1-phenylethanol (0.047 g, 0.16 mmol) in dichloromethane (1 mL) was added pyridinium chlorochromate (0.060 g, 0.28 mmol). The mixture was stirred at r.t. for 5 hours then filtered over celite, then washed several times with dichloromethane. The organic phase was evaporated till dryness. The residue was purified by flash chromatography over silica gel (Flashmart Pack: 5 g/60-40 um, eluent cyclohexane/ethyl acetate 4:1), yielding title compound (0.040 g, 38%) as a yellow solid;

mp: 159° C.; LC (XTerra RP₁₈, 3.5 μm, 3.0×50 mm Column): Rt=3.48 min; MS m/z (CI) [MH]⁺=298; ¹H NMR (300 MHz, CDCl₃) δ 2.50 (3H, d, 1.19 Hz), 2.55 (3H, s), 5.01 (2H, d, 4.11 Hz), 6.12 (1H, t, 4.11 Hz), 6.85 (1H, d, 1.19 Hz), 7.48 (2H, t, 7.42 Hz), 7.59 (1H, t, 7.42 Hz), 8.03 (2H, d, 8.45 Hz).

### Example 10

### 6-(methoxymethyl)-N-phenethylthieno[2,3-d]pyrimidin-4-amine (Final Compound 59)

### a) (4-(phenethylamino)thieno[2,3-d]pyrimidin-6-yl)methanol

According to Scheme 2 Step 1: To a solution of ethyl 4-(phenethylamino)thieno[2,3-d]pyrimidine-6-carboxylate (EXAMPLE 4—step c; 0.25 g, 0.76 mmol) in dry THF (10 mL) at 0° C. and under nitrogen atmosphere, was slowly added lithium aluminium hydride (0.087 g, 2.29 mmol). The mixture was stirred 6 h at that temperature and then allowed to warm to r.t. The mixture was hydrolyzed at 0° C. with water (80 μL), a 1M solution of sodium hydroxide (80 μL) and finally 240 mL of water were added. The mixture was then filtered through celite and washed with DCM. The organic layer was dried over MgSO4, filtered and evaporated till dryness. The residue was purified by flash chromatography over silica gel (Flashmart Pack: 50 g/60-40 um, eluent dichloromethane/methanol 98.5:1.5), yielding title compound (0.100 g, 46%) as a yellow oil.

### b) 6-(bromomethyl)-N-phenethylthieno[2,3-d]pyrimidin-4-amine

According to Scheme 2 Method A Step 2: To a solution of (4-(phenethylamino)thieno[2,3-d]pyrimidin-6-yl)methanol (0.33 g, 1.20 mmol) in THF (3 mL) at −10° C. and under vigorous stirring, was added triphenylphosphine (0.36 g, 1.39 mmol) and N-bromosuccinimide (0.25 mg, 1.39 mmol). The reaction mixture was stirred at that temperature 3 hours and then at r.t. overnight. The solvent was evaporated and the residue was purified by flash chromatography over silica gel (Flashmart Pack: 20 g/60-40 um, eluent dichloromethane/methanol 98:2), yielding title compound (0.02 g, 5%) as an oily yellow material.

### c) 6-(methoxymethyl)-N-phenethylthieno[2,3-d]pyrimidin-4-amine

According to Scheme 2 Method A Step 3: To a solution of 6-(bromomethyl)-N-phenethylthieno[2,3-d]pyrimidin-4-amine (0.02 g, 0.06 mmol) in methanol (0.5 mL) at 0° C. was slowly added a solution of sodium methoxide (from 0.3 g of sodium in 2.5 mL of dry methanol). The reaction mixture was stirred at 0° C. for 2 hours then allowed to warm to r.t. Water was then added and the mixture was extracted with dichloromethane. The organic layer was washed with brine, dried over MgSO4, filtered and evaporated till dryness. The residue was purified by flash chromatography over silica gel (Flashmart Pack: 5 g/60-40 um, eluent dichloromethane/methanol 99.5:0.5), yielding title compound (0.004 g, 23%) as a yellow oil; LC (XTerra RP₁₈, 3.5 μm, 3.0×50 mm Column): Rt=3.89 min; MS m/z (CI) [MH]⁺=300; ¹H NMR (300 MHz, CDCl₃) δ 2.94 (2H, t, 6.92 Hz), 3.35 (3H, s), 3.83 (2H, td, 6.57 Hz, 6.92 Hz), 4.56 (2H, s), 6.88 (1H, s), 7.14-7.32 (5H, m), 8.42 (1H, s).

### Example 11

### N-(4-(2-(2,6-dimethylthieno[2,3-d]pyrimidin-4-ylamino)ethyl)-phenyl)acetamide (Final compound 43)

### a) N-(4-aminophenethyl)-2,6-dimethylthieno[2,3-d]pyrimidin-4-amine

According to Scheme 7: Title compound was prepared according to EXAMPLE 2-step b, from 5-methyl-2-ethoxyethyleneamino-3-cyanothiophene (0.200 g, 0.960 mmol) and 2-(4-aminophenyl)ethylamine (0.392 g, 2.88 mmol) then crystallized in diisopropylether, yielding a brown solid (0.210 g, 73.3%) used without further purification.

### b) N-(4-(2-(2,6-dimethylthieno[2,3-d]pyrimidin-4-ylamino)ethyl)phenyl)acetamide

According to Scheme 12 Step 2: To a solution of N-(4-aminophenethyl)-2,6-dimethylthieno[2,3-d]pyrimidin-4-amine (0.050 g, 0.17 mmol) and triethylamine (0.047 ml, 0.34 mmol) in dichloromethane (15 mL) at 0° C., was slowly added acetyl chloride (0.012 ml, 0.17 mmol). The mixture was stirred at 0° C. for 3 hours and then water (10 mL) was added. The aqueous layer was extracted with dichloromethane, and then the organic layers were combined, dried over MgSO4, filtered, and evaporated till dryness. The residue (yellow solid, 0.148 g) was purified by chromatography over silica gel (Flashmart Pack: 20 g/60-40 um, eluent: dichloromethane/ethyl acetate 70:30 to pure ethyl acetate) and crystallized in pentane, yielding title compound (0.018 g, 32%), as a yellow solid;

mp: 248° C.; LC (XTerra RP₁₈, 3.5 μm, 3.0×50 mm Column): Rt=2.66 min; MS m/z (CI) [MH]⁺=341; ¹H NMR (300 MHz, CDCl₃) δ 2.19 (3H, s), 2.52 (3H, d), 2.61 (3H, s), 2.95 (2H, t, 6.91 Hz), 3.85 (2H, td, 6.72 Hz, 6.91 Hz), 6.61 (1H, m), 7.15 (1H, s), 7.18 (2H, d, 8.7 Hz), 7.45 (2H, d, 8.7 Hz).

### Example 12

### (4-(phenethylamino)thieno[2,3-d]pyrimidin-6-yl)methanol (Final Compound 58)

### a) ethyl 4-(phenethylamino)thieno[2,3-d]pyrimidine-6-carboxylate

According to Scheme 5 Method A Step 5: A mixture of ethyl 4-chlorothieno[2,3-d]pyrimidine-6-carboxylate (EXAMPLE 4—step c; 2.5 g, 10.3 mmol), phenethylamine (1.55 mL, 12.4 mmol) and potassium carbonate (2.14 g, 15.5 mmol) in acetonitrile (20 mL) were heated at 50° C. for 2 hours. The reaction mixture was filtered and the filtrate was washed with water and brine, dried over MgSO4, filtrated and concentrated till dryness, yielding title compound (3.11 g, 92%) as a solid.

### b) (4-(phenethylamino)thieno[2,3-d]pyrimidin-6-yl)methanol

According to Scheme 2 Step 1: To a solution of ethyl 4-(phenethylamino)thieno[2,3-d]pyrimidine-6-carboxylate (1.027 g, 3.14 mmol) in dry THF (20 mL) at 0° C. under nitrogen atmosphere, was added portionwise lithium aluminum hydride (190 mg, 7.84 mmol). The reaction mixture was stirred at that temperature for 6 hours and allowed to warm up to r.t. for 5 hours. The mixture was quenched at 0° C. by adding 400 uL of water, 400 uL of 1N sodium hydroxide solution and 1.2 mL of water, then filtered through celite, washed with dichloromethane. The organic layer was separated, dried over MgSO4, filtered and evaporated till dryness. The residue was purified by flash chromatography over silica gel (eluent dichloromethane/methanol 98:2), yielding 4-(phenethylamino)thieno[2,3-d]pyrimidine-6-carbaldehyde and title compound (0.150 g, 62%) as white solid;

mp: 155° C.; LC (XTerra RP₁₈, 3.5 μm, 3.0×50 mm Column): Rt=3.03 min; MS m/z (CI) [MH]⁺=286; ¹H NMR (300 MHz, CDCl₃) δ 3.00 (t, Jt=7.2 Hz, 2H), 3.89 (q, Jq=6.9 Hz, 2H), 4.87 (s, 2H), 6.92 (s, 1H), 7.22-7.35 (m, 5H), 8.49 (s, 1H).

### Example 13

### N-(4-((2H-tetrazol-5-yl)methoxy)phenethyl)-2,6-dimethylthieno[2,3-d]pyrimidin-4-amine (Final Compound 46)

### a) 2-(4-(2-(2,6-dimethylthieno[2,3-d]pyrimidin-4-ylamino)ethyl)phenoxy)acetonitrile

To a solution of 4-(2-(2,6-dimethylthieno[2,3-d]pyrimidin-4-ylamino)ethyl)phenol (EXAMPLE 2; 0.323 g, 1.08 mmol) in acetone (10 mL) at 0° C., was slowly added bromoacetonitrile (0.129 g, 1.08 mmol). The reaction mixture was stirred at 0° C. for 1 hour and then heated at 50° C. overnight. Solvent was evaporated and the residue was purified by chromatography over silica gel (eluent: dichloromethane/methanol 95:5), yielding title compound (0.323 g, 88%), as an orange solid; LC (XTerra RP₁₈, 3.5 μm, 3.0×50 mm Column): Rt=3.32 min; MS m/z (CI) [MH]⁺=339

### b) N-(4-((2H-tetrazol-5-yl)methoxy)phenethyl)-2,6-dimethylthieno[2,3-d]pyrimidin-4-amine

According to Scheme 11 Method A Step 1: A mixture of 2-(4-(2-(2,6-dimethylthieno[2,3-d]pyrimidin-4-ylamino)ethyl)phenoxy)acetonitrile (0.323 g, 0.95 mmol), azidotrimethylsilane (0.659 g, 5.72 mmol) and dibutyltinoxide (0.052 g, 0.21 mmol) in toluene (30 mL) was heated at 110° C. overnight. Solvent was removed under reduced pressure and the residue was taken up in dichloromethane and water. The aqueous phase was made acidic with a 1M hydrochloric acid solution and the precipitate was filtered off, washed with water and dried, yielding title compound (0.095 g, 26%) as a brown solid;

mp: 205° C.; LC (XTerra RP₁₈, 3.5 μm, 3.0×50 mm Column): Rt=2.88 min; MS m/z (CI) [MH]⁺=382; ¹H NMR (300 MHz, DMSO) δ 2.34 (3H, s), 2.41 (3H, s), 2.77 (2H, t, 7.21 Hz), 3.52 (2H, t, 7.21 Hz), 5.35 (2H, s), 6.89 (2H, d, 7.9 Hz), 7.1 (1H, s), 7.12 (2H, d, 7.9 Hz), 7.8 (1H, s).

### Example 14

### 6-isobutyl-N-phenethylthieno[2,3-d]pyrimidin-4-amine (Final Compound 95)

### a) 2-methyl-1-(4-(phenethylamino)thieno[2,3-d]pyrimidin-6-yl)propan-1-ol

To a solution of isopropyl magnesium bromide (0.13 mL of a 2M solution, 0.25 mmol) in THF (1 mL) at 0° C. was added dropwise a solution of 4-(phenethylamino)thieno[2,3-d]pyrimidine-6-carbaldehyde (from EXAMPLE 11—step d; 0.060 g, 0.21 mmol) in THF (1 mL). The reaction mixture was allowed to warm to r.t. and stirred at that temperature overnight. The reaction mixture was poured onto a saturated solution of ammonium chloride, then extracted with diethylether. The organic layer was washed with brine and water, dried over MgSO4, filtered and evaporated till dryness. The residue was purified by chromatography over silica gel (Flashmart Pack: 5 g/60-40 um, eluent: dichloromethane/methanol 99:1), yielding title compound (0.050 g, 75%).

### b) 6-isobutyl-N-phenethylthieno[2,3-d]pyrimidin-4-amine

To a solution of 2-methyl-1-(4-(phenethylamino)thieno[2,3-d]pyrimidin-6-yl)propan-1-ol (0.050 g, 0.15 mmol) in diethyl ether (6 ml) at r.t, was added portionwise aluminium chloride (0.14 g, 11.0 mmol) over 10 minutes. The reaction mixture was then cooled at 0° C. and lithium aluminium hydride (0.039 g, 1.0 mmol) was added portionwise over 5 minutes. The reaction mixture was stirred at 0° C. for 2 hours then at room temperature for 1 hour. Ethyl acetate (2 mL) was added at 0° C. to the mixture and after 5 minutes, water was added. The mixture was extracted with ethyl acetate and the organic layer was washed several times with water, dried over MgSO4, filtered and evaporated till dryness. The residue was purified by chromatography over silica gel (Flashmart Pack: 20 g/60-40 um, eluent: dichloromethane/methanol), yielding title compound (0.010 g, 21%), as a brown oil;

LC (XTerra RP₁₈, 3.5 μm, 3.0×50 mm Column): Rt=5.28 min; MS m/z (CI) [MH]⁺=312; ¹H NMR (300 MHz, CDCl₃) δ 0.88 (6H, d, 6.66 Hz), 1.86 (1H, m), 2.63 (2H, d, 7.17 Hz), 2.95 (2H, t, 7.04 Hz), 3.83 (2H, td, 6.32 Hz, 7.04 Hz), 5.91 (1H, s), 6.74 (1H, s), 7.17-7.28 (5H, m), 8.41 (1H, s).

The compounds in the following Tables 1 and 2 have been synthezised according to the previous examples, as denoted in the column denoted as “Ex. Nr”. The compound denoted with the asterisk has been exemplified in the Examples. When it concerns the bivalent linkers V₁ and V₂, it is noted that the left side of the linker V₁ as shown in the tables is attached to the thienyl-moiety and the left side of the linker V₂ as shown in the tables is attached to the pyrimidinyl-moiety.

TABLE 1
Pyrimidine-derivatives. c.b. = covalent bond

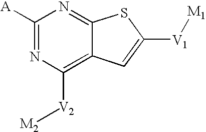

| Co.Nr. | Ex.Nr. | —V₁— | —M₁ | —V₂— | —M₂ | —A |
|---|---|---|---|---|---|---|
| 1 | 2 | — | — | 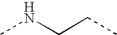 | 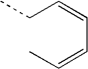 | —CH₃ |
| 2 | 2 | — | — | 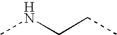 | 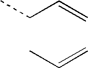 | —CH₂CH₃ |
| 3 | 2 | — | — | 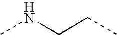 | 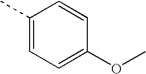 | — |
| 4 | 8 | —CH₂— | —H | c.b. | 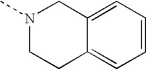 | —CH₃ |
| 5 | 2 | —CH₂— | —H | 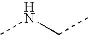 | 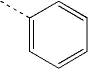 | —CH₃ |
| 6 | 2 | —CH₂— | —H | 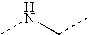 | 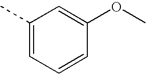 | —CH₃ |
| 7 | 2 | —CH₂— | —H | 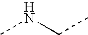 | 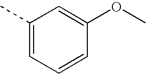 | —CH₂CH₃ |
| 8 | 8 | —CH₂— | —H | 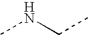 | 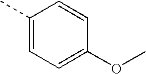 | —CH₃ |
| 9 | 2 | —CH₂— | —H | 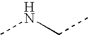 | 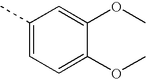 | —CH₃ |
| 10 | 2 | —CH₂— | —H | 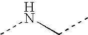 | 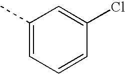 | —CH₃ |
| 11 | 8 | —CH₂— | —H | 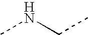 | 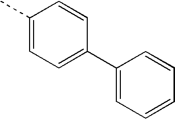 | —CH₃ |
| 12 | 9* | —CH₂— | —H | 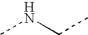 | 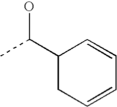 | —CH₃ |
| 13 | 2 | —CH₂— | —H | 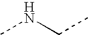 | 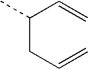 | —CH₃ |
| 14 | 7 | —CH₂— | —H | 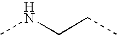 | 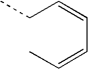 | —Cl |
| 15 | 7* | —CH₂— | —H | 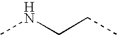 | 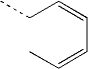 | —OCH₃ |
| 16 | 7 | —CH₂— | —H | 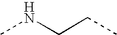 | 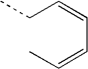 | —N(CH₃)₂ |
| 17 | 8 | —CH₂— | —H | 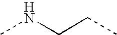 | 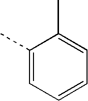 | —CH₃ |
| 18 | 8 | —CH₂— | —H | 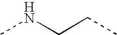 | 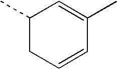 | —CH₃ |
| 19 | 2 | —CH₂— | —H | 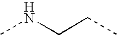 | 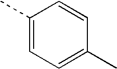 | —CH₃ |
| 20 | 8 | —CH₂— | —H | 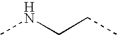 | 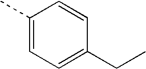 | —CH₃ |
| 21 | 2 | —CH₂— | —H | 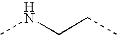 | 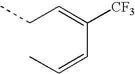 | —CH₃ |
| 22 | 8* | —CH₂— | —H | 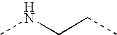 | 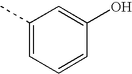 | —CH₃ |
| 23 | 2 | —CH₂— | —H | 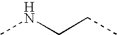 | 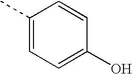 | —CH₃ |
| 24 | 2 | —CH₂— | —H | 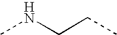 | 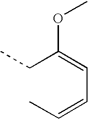 | —CH₃ |
| 25 | 2 | —CH₂— | —H | 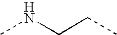 | 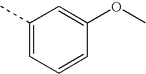 | —CH₃ |
| 26 | 2 | —CH₂— | —H | 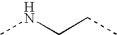 | 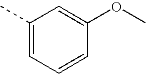 | —CH₂CH₃ |
| 27 | 2 | —CH₂— | —H | 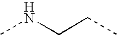 | 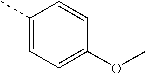 | — |
| 28 | 2 | —CH₂— | —H | 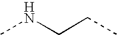 | 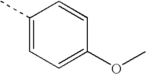 | —CH₃ |
| 29 | 2 | —CH₂— | —H | 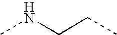 | 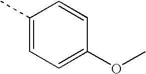 | —CH₂CH₃ |
| 30 | 8 | —CH₂— | —H | 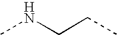 | 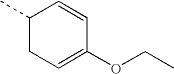 | —CH₃ |
| 31 | 8 | —CH₂— | —H | 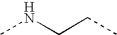 | 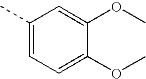 | —CH₃ |
| 32 | 2 | —CH₂— | —H | 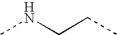 | 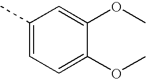 | — |
| 33 | 2 | —CH₂— | —H | 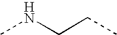 | 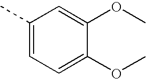 | —CH₃ |
| 34 | 2 | —CH₂— | —H | 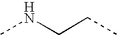 | 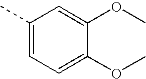 | —CH₂CH₃ |
| 35 | 8 | —CH₂— | —H | 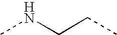 | 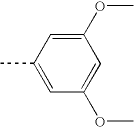 | —CH₃ |
| 36 | 8 | —CH₂— | —H | 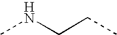 | 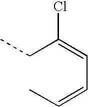 | —CH₃ |
| 37 | 8 | —CH₂— | —H | 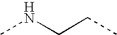 | 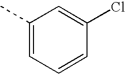 | —CH₃ |
| 38 | 2 | —CH₂— | —H | 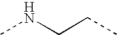 | 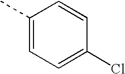 | —CH₃ |
| 39 | 8 | —CH₂— | —H | 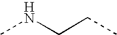 | 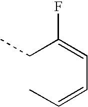 | —CH₃ |
| 40 | 8 | —CH₂— | —H | 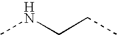 | 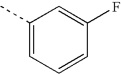 | —CH₃ |
| 41 | 2 | —CH₂— | —H | 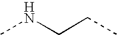 | 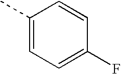 | —CH₃ |
| 42 | 2 | —CH₂— | —H | 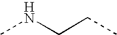 | 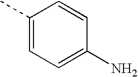 | —CH₃ |
| 43 | 11* | —CH₂— | —H | 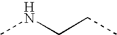 | 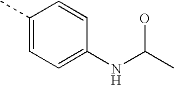 | —CH₃ |
| 44 | 8 | —CH₂— | —H | 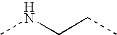 | 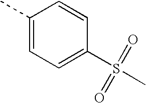 | —CH₃ |
| 45 | 8 | —CH₂— | —H | 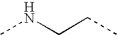 | 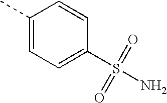 | —CH₃ |
| 46 | 13* | —CH₂— | —H | 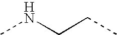 | 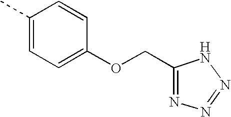 | —CH₃ |
| 47 | 8 | —CH₂— | —H | 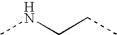 | 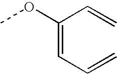 | —CH₃ |
| 48 | 2 | —CH₂— | —H | 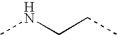 | 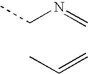 | —CH₃ |
| 49 | 8 | —CH₂— | —H | 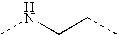 | 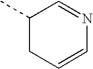 | —CH₃ |
| 50 | 8 | —CH₂— | —H | 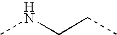 | 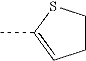 | —CH₃ |
| 51 | 3* | —CH₂— | —H | 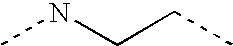 | 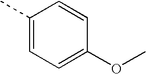 | —CH₃ |
| 52 | 2 | —CH₂— | —H | 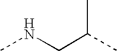 | 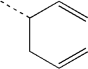 | —CH₃ |
| 53 | 2 | —CH₂— | —H | 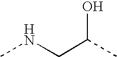 | 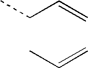 | —CH₃ |
| 54 | 8 | —CH₂— | —H | 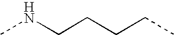 | 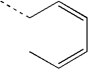 | —CH₃ |
| 55 | 5 | —CH₂— | —H | 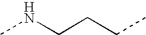 | 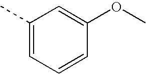 | —CH₃ |
| 56 | 5* | —CH₂— | —H | 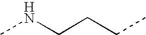 | 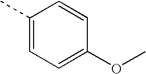 | —CH₃ |
| 57 | 7 | —CH₂— | —H | 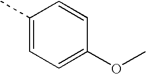 | —OCH₃ | —CH₃ |
| 58 | 12* | —CH₂— | —OH | 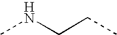 | 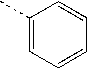 | — |
| 59 | 10* | —CH₂— | —OCH₃ | 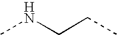 | 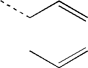 | — |
| 60 | 2 | —CH₂— | 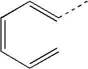 | 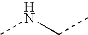 | 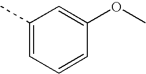 | —CH₃ |
| 61 | 2 | —CH₂— | 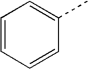 | 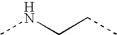 | 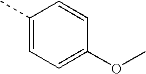 | —CH₃ |
| 62 | 4* | —C(═O)— | —N(CH₃)₂ | 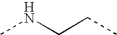 | 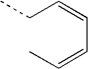 | |
| 63 | 8 | —CH₂CH₂— | —H | c.b. | 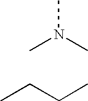 | — |
| 64 | 2 | —CH₂CH₂— | —H | 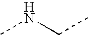 | 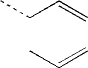 | — |
| 65 | 1 | —CH₂CH₂— | —H | 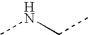 | 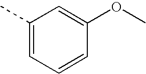 | — |
| 66 | 1 | —CH₂CH₂— | —H | 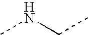 | 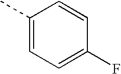 | — |
| 67 | 8 | —CH₂CH₂— | —H | 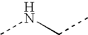 | 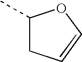 | — |
| 68 | 2 | —CH₂CH₂— | —H | 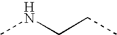 | 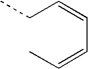 | — |
| 69 | 2 | —CH₂CH₂— | —H | 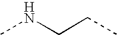 | 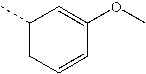 | |
| 70 | 2 | —CH₂CH₂— | —H | 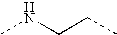 | 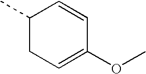 | — |
| 71 | 2 | —CH₂CH₂— | —H | 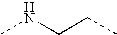 | 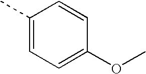 | —CH₃ |
| 72 | 2 | —CH₂CH₂— | —H | 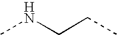 | 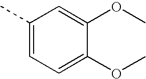 | — |
| 73 | 2 | —CH₂CH₂— | —H | 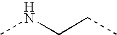 | 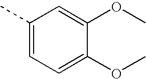 | —CH₃ |
| 74 | 1* | —CH₂CH₂— | —H | 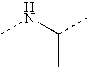 | 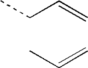 | — |
| 75 | 2 | —CH₂CH₂— | —H | 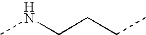 | 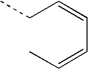 | — |
| 76 | 14 | —CH₂CH₂— | —H | 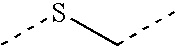 | —CN | — |
| 77 | 2 | —CH₂CH₂CH₂— | —H | 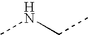 | 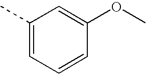 | — |
| 78 | 7 | —CH₂CH₂CH₂— | —H | 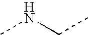 | 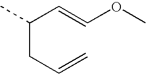 | —OCH₃ |
| 79 | 2* | —CH₂CH₂CH₂— | —H | 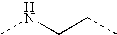 | 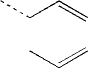 | — |
| 80 | 7 | —CH₂CH₂CH₂— | —H | 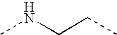 | 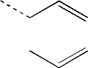 | —OCH₂CH₃ |
| 81 | 7 | —CH₂CH₂CH₂— | —H | 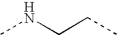 | 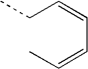 | —NH(CH₃) |
| 82 | 2 | —CH₂CH₂CH₂— | —H | 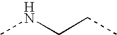 | 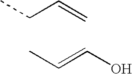 | — |
| 83 | 7 | —CH₂CH₂CH₂— | —H | 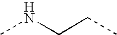 | 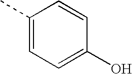 | —OCH₃ |
| 84 | 7 | —CH₂CH₂CH₂— | —H | 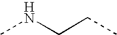 | 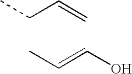 | —Cl |
| 85 | 2 | —CH₂CH₂CH₂— | —H | 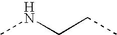 | 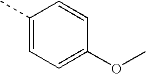 | — |
| 86 | 7 | —CH₂CH₂CH₂— | —H | 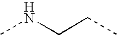 | 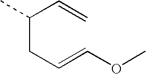 | —OCH₃ |
| 87 | 2 | —CH₂CH₂CH₂— | —H | 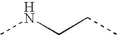 | 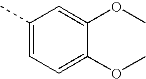 | — |
| 88 | 13 | —CH₂CH₂CH₂— | —H | 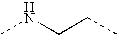 | 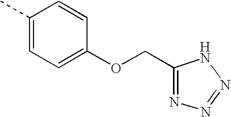 | —OCH₃ |
| 89 | 8 | —CH₂CH₂CH₂— | —H | 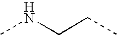 | 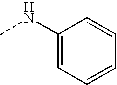 | — |
| 90 | 14 | —CH(OH)CH₂CH₂— | —H | 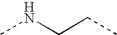 | 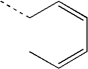 | — |
| 91 | 2 | —CH(CH₃)CH₂— | —H | 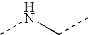 | 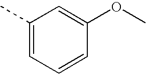 | — |
| 92 | 2 | —CH(CH₃)CH₂— | —H | 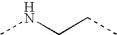 | 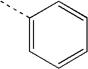 | — |
| 93 | 2 | —CH(CH₃)CH₂— | —H | 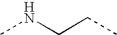 | 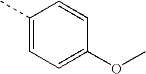 | — |
| 94 | 2 | —CH₂CH₂CH₂CH₂— | —H | 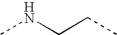 | 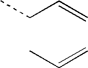 | — |
| 95 | 14* | —CH₂CH(CH₃)CH₂— | —H | 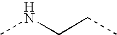 | 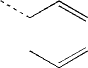 | — |

TABLE 2
Pyridine-derivatives

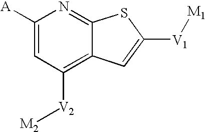

| Co.Nr. | Ex.Nr. | —V₁— | —M₁ | —V₂— | —M₂ | —A |
|---|---|---|---|---|---|---|
| 96 | 6* | —CH₂CH₂— | —H | 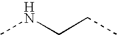 | 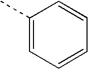 | — |

Physico-Chemical Data

Flash chromatography is a purification method well known to the practitioner skilled in organic chemistry. It is used in the context of the invention following conventional methods.

LCMS were recorded on a Waters Micromass ZQ 2996 system by the following conditions. Column 3.0*50 mm stainless steel packed with 5 μm XTerra RPC-18; flow rate 1 ml/min; mobile phase: A phase=0.1% formic acid in water, B phase=0.07% formic acid in acetonitrile. 0-0.5 min (A: 95%, B: 5%), 0.5-6.0 min (A: 0%, B: 100%), 6.0-6.5 min (A: 95%, B: 5%), 6.5-7 min (A: 95%, B: 5%); UV detection Diode Array: 200-400 nm; Injection volume: 3 μl. All mass spectra were taken under electrospray ionisation (ESI) methods. Table 3 shows patent peak (MW) and retention time (RT, in minutes).

Most of the reaction were monitored by thin-layer chromatography on 0.25 mm Macherey-Nagel silica gel plates (60F-2254), visualized with UV light. Flash column chromatography was performed on silica gel (220-440 mesh, Fluka).

Melting point determination was performed on a Buchi B-540 apparatus.

¹H NMR spectra were recorded on a Brucker 500 MHz. Chemical shifts are expressed in parts of million (ppm, δ units). Coupling constants are in units of hertz (Hz). Splitting patterns describe apparent multiplicities and are designated as s (singulet), d (doublet), t (triplet), q (quartet), quint (quintet), m (multiplet). Table 4 shows the NMR-data.

TABLE 3
Melting point and chromatography data

| | Melting point | | RT | |
|---|---|---|---|---|
| Co. Nr | (° C.) | [MH⁺] | (min) | Physical form |
| 1 | 111 | 270 | 3.12 | solid |
| 2 | 89 | 284 | 3.43 | solid |
| 3 | — | 286 | 3.58 | solid |
| 4 | 105 | 296 | 3.8 | solid |
| 5 | 124.2 | 270 | 3.16 | crystals |
| 6 | 97 | 300 | 3.21 | crystals |
| 7 | 108.4 | 314 | 3.48 | cristals |
| 8 | 127.5 | 300 | 3.14 | solid |
| 9 | 142 | 330 | 2.91 | crystals |
| 10 | 136.5 | 304-306 | 3.69 | yellow crystals |
| 11 | 150-152 | 346 | 4.64 | solid |
| 12 | 159 | 298 | 3.48 | yellow solid |
| 13 | 106 | 284 | 3.23 | crystals |
| 14 | 119 | 304-306 | 4.93 | solid |
| 15 | 138.5 | 300 | 4.45 | solid |
| 16 | 142 | 313 | 3.22 | solid |
| 17 | 150.4-152.2 | 298 | 3.55 | solid |
| 18 | 147.8-148.5 | 298 | 3.56 | solid |
| 19 | 115 | 298 | 3.51 | crystals |
| 20 | 62 | 312 | 3.91 | solid |
| 21 | 98.2 | 352 | 3.76 | brown crystals |
| 22 | 162.5 | 300 | 2.6 | solid |
| 23 | 166 | 300 | 2.52 | powders |
| 24 | 134 | 314 | 3.32 | crystals |
| 25 | 139 | 314 | 3.22 | crystals |
| 26 | 83.5 | 328 | 3.48 | cristals |
| 27 | 199 | 300 | 3.81 | solid |
| 28 | 129 | 314 | 3.21 | crystals |
| 29 | 82 | 328 | 3.43 | solid |
| 30 | 101-103 | 328 | 3.48 | solid |
| 31 | 135 | 330 | 2.77 | yellow solid |
| 32 | 124 | 330 | 2.81 | solid |
| 33 | 85 | 344 | 2.88 | crystals |
| 34 | 105 | 358 | 3.14 | solid |
| 35 | 106-107 | 344 | 3.34 | solid |
| 36 | 142.2-144.4 | 318-320 | 3.8 | solid |
| 37 | 161-162 | 318-320 | 3.74 | solid |
| 38 | 129 | 318-320 | 3.61 | crystals |
| 39 | 121-122 | 302 | 3.41 | solid |
| 40 | 131-132 | 302-304 | 3.56 | solid |
| 41 | 117.2 | 302 | 3.38 | yellow crystals |
| 42 | 127 | 299 | 1.82-1.98 | solid |
| 43 | 248 | 341 | 2.66 | solid |
| 44 | 176-179.5 | 362 | 2.77 | solid |
| 45 | decomp at 270 | 363 | 2.55 | solid |
| 46 | 205 | 382 | 2.88 | beige solid |
| 47 | 156.5 | 300 | 3.38 | solid |
| 48 | 139 | 285 | 0.73 | yellow crystals |
| 49 | 138 | 285 | 1.81 | solid |
| 50 | 102.5-103.7 | 290 | 3.23 | solid |
| 51 | 81 | 328 | 3.63 | crystals |
| 52 | 94 | 298 | 3.43 | white crystals |
| 53 | 117 | 300 | 2.65 | solid |
| 54 | 108 | 312 | 3.85 | solid |
| 55 | 122 | 328 | 3.28 | solid |
| 56 | 120 | 328 | 3.23 | white solid |
| 57 | 90 | 252 | 2.19 | crystals |
| 58 | 155-155.5 | 286 | 3.03 | white powder |
| 59 | — | 300 | 3.89 | brown oil |
| 60 | 107 | 376 | 4.14 | crystals |
| 61 | — | 390 | 4.11 | visceous oil |
| 62 | 157 | 327 | 3.63 | yellow solid |
| 64 | 133 | 270 | 3.58 | solid |
| 65 | 99 | 300 | 4.19 | solid |
| 66 | 104 | 288 | 4.34 | solid |
| 67 | — | 260 | 3.78 | solid |
| 68 | 118 | 284 | 4.23 | pale yellow crystals |
| 69 | 81 | 314 | 4.36 | crystals |
| 70 | 133 | 314 | 4.33 | crystals |
| 71 | 102 | 328 | 3.44 | yellow crystals |
| 72 | 124 | 344 | 3.14 | solid |
| 73 | 78 | 358 | 3.19 | yellow crystals |
| 74 | 141 | 284 | 4.48 | solid |
| 75 | 124.5 | 298 | 4.46 | white crystals |
| 77 | 118 | 314 | 4.56 | crystals |
| 78 | — | 358 | 5.14 | yellow oil |
| 79 | 110.5 | 298 | 4.71 | crystals |
| 80 | — | 342 | 5.35 | yellow oil |
| 81 | 162 | 327 | 4.4 | white solid |
| 82 | 123.5 | 314 | 3.67 | solid |
| 83 | 79 | 344 | 4.3 | white solid |
| 84 | 135 | 348 | 4.7 | yellow solid |
| 85 | 102 | 328 | 4.58 | crystals |
| 86 | 92 | 358 | 5.05 | yellow solid |
| 87 | 128.5 | 358 | 4.18 | solid |
| 88 | 181 | 426 | 4.37 | yellow solid |
| 89 | 68 | 313 | 4.49 | brown solid |
| 90 | — | 314 | 3.6 | yellow oil |
| 91 | 65 | 314 | 4.53 | crystals |
| 92 | 117.5 | 298 | 4.59 | crystals |
| 93 | 108.5 | 328 | 4.49 | crystals |
| 94 | 95 | 312 | 5.25 | solid |
| 95 | — | 312 | 5.28 | brown oil |
| 96 | 88 | 283 | 3.19 | solid |

TABLE 4
NMR-data for selected compounds

| Co. | |
|---|---|
| Nr | NMR-data |
| 1 | ¹H NMR(300MHz, CDCl₃) δ 2.63(3H, s), 2.99(2H, t, 6.92Hz), 3.89(2H, td, 6.14Hz, 6.92Hz), 5.09(1H, t, 6.14Hz), 6.96(1H, d, 6.01Hz), |
| | 7.14(1H, d, 6.01Hz), 7.25(3H, m), 7.34(2H, m) |
| 2 | ¹H NMR(300MHz, CDCl₃) δ 1.39(3H, t, 7.55Hz), 2.89(2H, q, 7.55Hz), 3.01(2H, t, 7.04Hz), 3.89(2H, td, 5.89Hz, 7.04Hz), 5.13(1H, s), |
| | 6.97(1H, d, 6.14Hz), 7.14(1H, d, 6.14Hz), 7.24-7.36(5H, m) |
| 3 | ¹H NMR(500MHz, CDCl₃) δ 2.96(2H, t, 6.82Hz), 3.81(3H, s), 3.87(2H, td, 6.48Hz, 6.82Hz), 5.17(1H, m), 6.88(2H, d, 8.65Hz), |
| | 7.02(1H, d, 5.99Hz), 7.17(2H, d, 8.65Hz), 7.28(1H, d, 5.99Hz), 8.54(1H, s) |
| 4 | ¹H NMR(300MHz, CDCl₃) δ 2.55(3H, d, 1.28Hz), 2.6(3H, s), 3.05(2H, t, 5.89Hz), 4.08(2H, t, 5.89Hz), 4.98(2H, s), 7.04(1H, d, 1.28Hz), |
| | 7.22(4H, m) |
| 5 | ¹H NMR(500MHz, CDCl₃) δ 2.53(3H, d, 1.08Hz), 2.62(3H, s), 4.83(2H, d, 5.49Hz), 5.13(1H, s), 6.71(1H, d, 1.08Hz), |
| | 7.35-7.40(5H, m) |
| 6 | ¹H NMR(500MHz, CDCl₃) δ 2.53(3H, d, 1.07Hz), 2.62(3H, s), 3.82(3H, s), 4.8(2H, d, 5.47Hz), 5.11(1H, s), 6.85(1H, dd, 7.24Hz, |
| | 8.2Hz), 6.95(1H, s), 6.97(1H, d, 7.24Hz), 7.29(1H, d, 8.2Hz) |
| 7 | ¹H NMR(500MHz, CDCl₃) δ 1.37(t, J=7.6Hz, 3H), 2.53(d, J=1.2Hz, 3H), 2.87(q, J=7.6Hz, 2H), 3.81(s, 3H), 4.82(d, J=5.5Hz, 2H), |
| | 5.15(m, J=5.5Hz, 1H), 6.70(d, J=1.2Hz, 1H), 6.85(q, 1H), 6.97(m, 2H), 7.27(d, 1H) |
| 9 | 1H NMR(500MHz, CDCl3) d 2.53(3H, d, 1.11Hz), 2.63(3H, s), 3.88(3H, s), 3.9(3H, s), 4.74(2H, d, 5.36Hz), 5.09(1H, s), 6.7(1H, d, |
| | 1.11Hz), 6.86(1H, d, 8.1Hz), 6.94(1H, dd, 1.95Hz, 8.1Hz), 6.96(1H, d, 1.95Hz) |
| 10 | 1H NMR(500MHz, CDCl3) d 2.54(3H, d, 1.21Hz), 2.61(3H, s), 4.82(2H, d, 5.72Hz), 5.17(1H, s), 6.72(1H, d, 1.21Hz), 7.28(3H, m), |
| | 7.38(1H, s) |
| 12 | ¹H NMR(300MHz, CDCl₃) δ 2.50(3H, d, 1.19Hz), 2.55(3H, s), 5.01(2H, d, 4.11Hz), 6.12(1H, t, 4.11Hz), 6.85(1H, d, 1.19Hz), 7.48(2H, t, |
| | 7.42Hz), 7.59(1H, t, 7.42Hz), 8.03(2H, d, 8.45Hz) |
| 13 | ¹H NMR(500MHz, CDCl₃) δ 2.52(3H, d, 1.07Hz), 2.61(3H, s), 2.98(2H, t, 6.91Hz), 3.88(2H, td, 5.87Hz, 6.91Hz), 4.92(1H, s), |
| | 6.59(1H, d, 1.07Hz), 7.26(3H, m), 7.35(2H, m) |
| 14 | ¹H NMR(300MHz, CDCl₃) δ 2.52(3H, d, 1.28Hz), 2.98(2H, t, 6.79Hz), 3.87(2H, td, 5.89Hz, 6.79Hz), 5.12(1H, s), 5.59(1H, d, |
| | 1.28Hz), 7.22-7.37(5H, m) |
| 15 | ¹H NMR(300MHz, CDCl₃) δ 2.4(3H, d, 1.02Hz), 2.9(2H, t, 6.66Hz), 3.78(2H, td, 5.89Hz, 6.66Hz), 3.93(3H, s), 4.91(1H, s), 6.45(1H, d, |
| | 1.02Hz), 7.17(3H, m), 7.26(2H, m) |
| 16 | ¹H NMR(300MHz, CDCl₃) δ 2.42(3H, s), 2.97(2H, t, 7.04Hz), 3.21(6H, s), 3.79(2H, td, 6.4Hz, 7.04Hz), 4.78(1H, s), 6.43(1H, s), |
| | 7.24(3H, m), 7.31(2H, m) |
| 19 | ¹H NMR(500MHz, CDCl₃) δ 2.36(3H, s), 2.52(3H, d, 1.12Hz), 2.61(3H, s), 2.94(2H, t, 6.87Hz), 3.85(2H, td, 6.87Hz, 6.87Hz), |
| | 4.89(1H, s), 6.6(1H, d, 1.12Hz), 7.15(4H, 2d, 1.51Hz) |
| 21 | 1H NMR(500MHz, CDCl3) d 2.53(3H, s), 2.62(3H, s), 3.05(2H, t, 6.98Hz), 3.89(2H, td, 6.98Hz, 6.98Hz), 4.95(1H, s), 6.62(1H, s), |
| | 7.44(2H, m), 7.51(2H, m) |
| 22 | ¹H NMR(300MHz, CDCl₃) δ 2.51(3H, s), 2.59(3H, s), 2.93(2H, t, 6.61Hz), 3.85(2H, td, 6.01Hz, 6.61Hz), 6.64(1H, s), 6.73-6.81(3H, m), |
| | 7.19(1H, dd, 7.8Hz, 8.4Hz) |
| 23 | ¹H NMR(500MHz, DMSO) δ 2.4(3H, s), 2.47(3H, d, 1.17Hz), 2.77(2H, t, 7.54Hz), 3.57(2H, td, 5.47Hz, 7.54Hz), 6.66(2H, d, 8.47Hz), |
| | 7.03(2H, d, 8.47Hz), 7.16(1H, d, 1.17Hz), 7.67(1H, t, 5.47Hz), 9.16(1H, s) |
| 24 | ¹H NMR(500MHz, CDCl₃) δ 2.52(3H, d, 1.11Hz), 2.59(3H, s), 3.01(2H, t, 6.58Hz), 3.81(2H, td, 5.35Hz, 6.58Hz), 3.89(3H, s), |
| | 5.22(1H, s), 6.6(1H, d, 1.11Hz), 6.91(1H, d, 8.52Hz), 6.94(1H, dd, 8.05Hz, 7.39Hz), 7.17(1H, d, 7.39Hz), 7.25(1H, dd, 8.05Hz, 8.52Hz) |
| 25 | ¹H NMR(500MHz, CDCl₃) δ 2.52(3H, d, 1.12Hz), 2.61(3H, s), 2.95(2H, t, 6.86Hz), 3.8(3H, s), 3.87(2H, td, 5.88Hz, 6.86Hz), |
| | 4.9(1H, s), 6.6(1H, d, 1.12Hz), 6.78-6.85(2H, m), 7.26(1H, dd) |
| 26 | ¹H NMR(500MHz, CDCl₃) δ 1.39(t, J=7.6Hz, 3H), 2.52(s, 3H), 2.85(q, J=7.6Hz, 2H), 2.97(t, 6.9Hz, 2H), 3.80(s, 3H), 3.88(m, |
| | J=6.9Hz, 2H), 4.95(s, 1H), 6.6(s, 1H), 6.82(m, 4H) |
| 27 | ¹H NMR(500MHz, CDCl₃) δ 2.55(d, J=1.2Hz, 3H), 2.95(t, J=6.8Hz, 2H), 3.82(s, 3H), 3.89(t, J=6.8Hz, 2H), 5.0(s, 1H), 6.6(m, |
| | J=1.2Hz, 1H), 6.90(d, J=8.7Hz, 2H), 7.25(d, J=8.7Hz, 2H), 8.45(s, 1H) |
| 28 | ¹H NMR(500MHz, CDCl₃) δ 2.52(3H, d, 1.04Hz), 2.60(3H, s), 2.92(2H, t, 6.86Hz), 3.82(3H, s), 3.84(2H, td, 6.86Hz, 6.86Hz), 4.88(1H, s), |
| | 6.59(1H, d, 1.04Hz), 6.88(2H, d, 8.57Hz), 7.16(2H, d, 8.57Hz) |
| 29 | ¹H NMR(500MHz, CDCl₃) δ 1.37(t, J=7.6Hz, 3H), 2.53(d, J=1.1Hz, 3H), 2.86(q, J=7.6Hz, 2H), 2.93(t, J=6.9Hz, 2H), 3.82(s, 3H), |
| | 3.85(d, J=6.9Hz, 2H), 4.59(s, 1H), 6.6(d, J=1.1Hz, 1H), 6.90(m, 2H), 7.15(m, 2H) |
| 33 | 1H NMR(500MHz, CDCl3) d 2.52(3H, s), 2.61(3H, s), 2.92(2H, t, 6.78Hz), 3.85(3H, s), 3.86(2H, td, 6.78Hz, 6.78Hz), 3.89(3H, s), 4.93(1H, s), |
| | 6.59(1H, s), 6.74(1H, d, 1.87Hz), 6.78(1H, dd, 1.87Hz, 8.1Hz), 6.84(1H, d, 8.1Hz) |
| 34 | ¹H NMR(500MHz, CDCl₃) δ 1.38(t, J=7.6Hz, 3H), 2.53(d, J=1.0Hz 3H), 2.88(q, J=7.6Hz, 2H), 2.94(t, 2H), |
| | 3.87(m, 8H), 5.20(t, 1H), 6.60(m, J=1.0Hz 1H), 6.77(s, 1H), 6.80(dd, J=8.1Hz, 1H), 6.85(d, J=8.1Hz, 1H) |
| 38 | 1H NMR(500MHz, CDCl3) d 2.53(3H, d, 1.16Hz), 2.61(3H, s), 2.96(2H, t, 6.91Hz), 3.85(2H, td, 6.91Hz, 6.91Hz), |
| | 4.89(1H, s), 6.6(1H, d, 1.16Hz), 7.17(2H, d, 8.42Hz), 7.3(2H, d, 8.42Hz) |
| 39 | ¹H NMR(300MHz, CDCl₃) δ 2.45(3H, d, 1.05Hz), 2.52(3H, s), 2.96(2H, t, 6.91Hz), 3.79(2H, td, 6.91Hz, 6.47Hz), 5.05(1H, s), |
| | 6.59(1H, d, 1.05Hz), 6.95-7.18(4H, m) |
| 41 | 1H NMR(500MHz, CDCl3) d 2.53(3H, d, 1.12Hz), 2.61(3H, s), 2.96(2H, t, 6.78Hz), 3.85(2H, td, 6.78Hz, 6.78Hz), 4.9(1H, s), |
| | 6.6(1H, d, 1.12Hz), 7.02(2H, dd, 8.69Hz, 8.69Hz), 7.2(2H, dd, 5.4Hz, 8.69Hz) |
| 42 | ¹H NMR(300MHz, CDCl₃) δ 2.51(3H, d, 1.02Hz), 2.59(3H, s), 2.85(2H, t, 6.92Hz), 3.79(2H, td, 5.89Hz, 6.92Hz), 4.92(1H, s), 6.58(1H, d, |
| | 1.02Hz), 6.67(1H, d, 8.19Hz), 7.02(1H, d, 8.19Hz) |
| 43 | ¹H NMR(300MHz, CDCl₃) δ 2.19(3H, s), 2.52(3H, d), 2.61(3H, s), 2.95(2H, t, 6.91Hz), 3.85(2H, td, 6.72Hz, 6.91Hz), 6.61(1H, m), |
| | 7.15(1H, s), 7.18(2H, d, 8.7Hz), 7.45(2H, d, 8.7Hz) |
| 45 | ¹H NMR(300MHz, CDCl₃) δ 2.34(3H, s), 2.41(3H, d, 1.28Hz), 2.91(2H, t, 7.04Hz), 3.61(2H, td, 6.48Hz, 7.04Hz), 5.23(1H, t, 6.48Hz), |
| | 7.09(1H, d, 1.28Hz), 7.35(2H, d, 8.19Hz), 7.65(2H, d, 8.19Hz), 7.83(2H, m) |
| 46 | ¹H NMR(300MHz, DMSO) δ 2.34(3H, s), 2.41(3H, s), 2.77(2H, t, 7.21Hz), 3.52(2H, t, 7.21Hz), 5.35(2H, s), 6.89(2H, d, 7.9Hz), 7.1(1H, s), |
| | 7.12(2H, d, 7.9Hz), 7.8(1H, s) |
| 47 | ¹H NMR(300MHz, CDCl₃) δ 2.53(3H, d, 1.02Hz), 2.61(3H, s), 4.03(2H, td, 5.12Hz, 5.38Hz), 4.2(2H, t, 5.12Hz), 5.37(1H, t, 5.38Hz), |
| | 6.74(1H, d, 1.02Hz), 6.97(3H, m), 7.29(2H, m) |
| 48 | 1H NMR(500MHz, CDCl3) d 2.54(3H, d, 1.15Hz), 2.59(3H, s), 3.15(2H, t, 6.23Hz), 4(2H, td, 6.23Hz, 6.23Hz), 6.32(1H, s), 6.73(1H, d, |
| | 1.15Hz), 7.2(1H, dd, 4.97Hz, 7.59Hz), 7.22(1H, d, 7.76Hz), 7.64(1H, dd, 7.59Hz, 7.76Hz), 8.6(1H, d, 4.97Hz) |
| 50 | ¹H NMR(300MHz, CDCl₃) δ 2.45(3H, d, 1.07Hz), 2.54(3H, s), 3.13(2H, t, 6.55Hz), 3.82(2H, td, 6.33Hz, 6.55Hz), 5.05(1H, s), 6.58(1H, d, |
| | 1.07Hz), 6.81(1H, d, 3.31Hz), 6.9(1H, dd, 5.11Hz, 3.31Hz), 7.11(1H, d, 5.11Hz) |
| 51 | ¹H NMR(500MHz, CDCl₃) δ 2.52(3H, d, 1.04Hz), 2.58(3H, s), 2.94(2H, t, 7.61Hz), 3.28(3H, s), 3.81(3H, s), 3.92(2H, t, 7.61Hz), |
| | 6.86(2H, d, 8.57Hz), 7(1H, d, 1.04Hz), 7.17(2H, d, 8.57Hz) |
| 52 | ¹H NMR(500MHz, CDCl₃) δ 1.37(3H, d, 6.99Hz), 2.5(3H, d, 1.01Hz), 2.61(3H, s), 3.12(1H, m), 3.57(1H, m), 3.99(1H, m), 4.75(1H, s), |
| | 6.49(1H, d, 1.01Hz), 7.28(3H, m), 7.37(2H, m) |
| 53 | ¹H NMR(300MHz, DMSO) δ 2.43(3H, s), 2.52(3H, s), 3.74(2H, m), 4.89(1H, m), 5.62(1H, d, 4.51Hz), 7.23(1H, s), 7.32-7.42(5H, m), |
| | 7.77(1H, t, 5.94Hz) |
| 54 | ¹H NMR(300MHz, CDCl₃) δ 1.72(4H, m), 2.52(3H, d, 1.02Hz), 2.58(3H, s), 2.69(2H, t, 7.17Hz), 3.62(2H, td, 5.63Hz, 7.17Hz), |
| | 4.83(1H, s), 6.68(1H, d, 1.02Hz), 7.19(3H, m), 7.29(2H, m) |
| 55 | ¹H NMR(300MHz, CDCl₃) δ 2.02(2H, tt, 7.3Hz, 7.43Hz), 2.52(3H, d, 1.28Hz), 2.57(3H, s), 2.74(2H, t, 7.43Hz), 3.65(2H, td, 5.89Hz, |
| | 7.3Hz), 3.79(3H, s), 4.72(1H, s), 6.54(1H, d, 1.28Hz), 6.75-6.84(3H, m), 7.22(1H, dd, 7.57Hz, 8.96Hz) |
| 56 | ¹H NMR(300MHz, CDCl₃) δ 1.99(2H, tt, 7.17Hz, 7.43Hz), 2.51(3H, d, 1.02Hz), 2.57(3H, s), 2.71(2H, t, 7.43Hz), 3.64(2H, td, 5.63Hz, |
| | 7.17Hz), 3.8(3H, s), 4.75(1H, s), 6.5(1H, d, 1.02Hz), 6.84(2H, d, 8.71Hz), 7.14(2H, d, 8.71Hz) |
| 57 | ¹H NMR(500MHz, CDCl₃) δ 1.95(2H, tt, 5.61Hz, 5.97Hz), 2.57(3H, d, 1.07Hz), 2.61(3H, s), 3.44(3H, s), 3.62(2H, t, 5.61Hz), 3.71(2H, td, |
| | 5.42Hz, 5.97Hz), 5.71(1H, s), 6.68(1H, d, 1.07Hz) |
| 58 | ¹H NMR(300MHz, CDCl₃) δ 2.93(2H, t, 6.65Hz), 3.82(2H, td, 6.65Hz, 5.7Hz), 4.8(2H, s), 5.12-5.23(1H, m), 6.84(1H, s), 7.18-7.26(5H, m), |
| | 8.42(1H, s) |
| 59 | ¹H NMR(300MHz, CDCl₃) δ 2.94(2H, t, 6.92Hz), 3.35(3H, s), 3.83(2H, td, 6.57Hz, 6.92Hz), 4.56(2H, s), 6.88(1H, s), 7.14-7.32(5H, m), |
| | 8.42(1H, s) |
| 60 | ¹H NMR(500MHz, CDCl₃) δ 2.61(s, 3H), 3.81(s, 3H), 4.16(s, 2H), 4.77(d, J=5.6Hz, 2H), 5.15(t, J=5.6Hz, 1H), 6.64(s, 1H), |
| | 6.95(m, 3H), 7.30(m, 6H) |
| 61 | ¹H NMR(500MHz, CDCl₃) δ 2.60(s, 3H), 2.90(t, J=7.0Hz, 2H), 3.78-3.83(massive, J=7.0Hz, 5H), 4.15(s, 2H), 5.00(s, 1H), 6.57(s, |
| | 1H), 6.86(d, J=8.6Hz, 2H), 7.15(d, J=8.6Hz, 2H), 7.28(m, 3H), 7.35(m, 2H) |
| 62 | ¹H NMR(300MHz, CDCl₃) δ 1.18(6H, s), 2.95(2H, t), 3.85(2H, td), 5.71(1H, m), 7.17-7.29(5H, m), 7.37(1H, s), 8.46(1H, s) |
| 65 | ¹H NMR(500MHz, CDCl₃) δ 1.36(t, J=7.5Hz, 3H), 2.91(q, J=7.5Hz, 2H), 3.81(s, 3H), 4.81(d, J=5.5Hz, 2H), 5.38(t, J=5.5Hz, 1H)), |
| | 6.8(s, 1H), 6.86(dd, J=2.1Hz, J=8.2Hz, 1H), 6.94(m, J=2.1Hz, 1H), 6.98(d, J=7.5Hz, 1H), 7.29(dd, J=7.5Hz, J=2.1Hz |
| 68 | 1H NMR(500MHz, CDCl3) d 1.36(3H, t, 7.52Hz), 2.9(2H, qd, 1.12Hz, 7.52Hz), 3(2H, t, 6.86Hz), 3.89(2H, td, 6.86Hz, 6.86Hz), 5.02(1H, s), |
| | 6.66(1H, t, 1.12Hz), 7.26(3H, m), 7.35(2H, m), 8.48(1H, s) |
| 69 | ¹H NMR(500MHz, CDCl₃) δ 1.36(3H, t, 7.53Hz), 2.89(2H, qd, 1.07Hz, 7.52Hz), 2.98(2H, t, 6.84Hz), 3.8(3H, s), 3.89(2H, td, 5.94Hz, |
| | 6.84Hz), 5.21(1H, s), 6.69(1H, d, 1.07Hz), 6.78-6.86(3H, m), 7.26(1H, dd), 8.47(1H, s) |
| 70 | ¹H NMR(500MHz, CDCl₃) δ 1.36(3H, t, 7.52Hz), 2.89(2H, qd, 1.07Hz, 7.52Hz), 2.93(2H, t, 6.85Hz), 3.82(3H, s), 3.85(2H, td, 5.87Hz, |
| | 6.85Hz), 5.01(1H, s), 6.66(1H, d, 1.07Hz), 6.89(2H, d, 8.63Hz), 7.16(2H, d, 8.63Hz), 8.47(1H, s) |
| 71 | 1H NMR(500MHz, CDCl3) d 1.34(3H, t, 7.52Hz), 2.61(3H, s), 2.87(2H, dq, 0.95Hz, 7.52Hz), 2.93(2H, t, 6.86Hz), 3.82(3H, s), 3.84(2H, td, |
| | 6.86Hz, 6.86Hz), 4.93(1H, s), 6.62(1H, s), 6.88(2H, d, 8.6Hz), 7.17(2H, d, 8.6Hz) |
| 72 | ¹H NMR(500MHz, CDCl₃) δ 1.35(t, J=7.6Hz, 3H), 2.89(q, J=7.6Hz, 2H), 2.94(t, J=6.8Hz, 2H), 3.84(s, 3H), 3.86(t, J=6.8Hz, 2H), |
| | 3.89(s, 3H), 5.15(s, 1H), 6.65(s, 1H), 6.75(d, J=1.9Hz, 1H), 6.80(dd, J=1.9Hz, J=8.1Hz, 1H), 6.85(d, J=8.1Hz, 1H), 8.45(s, 1H) |
| 73 | 1H NMR(500MHz, CDCl3) d 1.34(3H, t, 7.52Hz), 2.62(3H, s), 2.87(2H, qd, 1.08Hz, 7.52Hz), 2.93(2H, t, 6.89Hz), 3.85(3H, s), 3.86(2H, td, |
| | 6.86Hz, 6.86Hz), 3.89(3H, s), 4.93(1H, s), 6.61(1H, s), 6.74(1H, d, 1.87Hz), 6.79(1H, dd, 1.87Hz, 8.12Hz), 6.85(1H, d, 8.12Hz) |
| 74 | ¹H NMR(500MHz, CDCl₃) δ 1.36(3H, t, 7.52Hz), 1.66(3H, d, 6.85Hz), 2.9(2H, qd, 7.52Hz, 1.08Hz), 5.26(1H, m), 5.6(1H, qd, 6.85Hz, |
| | 7.09Hz), 6.8(1H, d, 1.08Hz), 7.29(1H, m), 7.37(2H, m), 7.42(2H, m), 8.44(1H, s) |
| 75 | 1H NMR(500MHz, CDCl3) d 1.37(3H, t, 7.52Hz), 2.06(2H, tt, 7.18Hz, 7.43Hz), 2.78(2H, t, 7.43Hz), 2.9(2H, dq, 1.13Hz, 7.52Hz), 3.68(2H, td, |
| | 7.43Hz, 7.18Hz), 4.89(1H, s), 6.6(1H, t, 1.13Hz), 7.25(3H, m), 7.32(2H, m), 8.44(1H, s) |
| 77 | ¹H NMR(500MHz, CDCl₃) δ 1.01(3H, t, 7.34Hz), 1.75(2H, tq, 7.34Hz, 7.84Hz), 2.85(2H, t, 7.84Hz), 3.81(3H, s), 4.8(2H, d, 5.51Hz), |
| | 5.24(1H, s), 6.78(1H, s), 6.86(1H, d, 8.23Hz), 6.94(1H, s), 6.98(1H, d, 7.56Hz), 7.29(1H, dd, 7.56Hz, 8.23Hz), 8.50(1H, s) |
| 78 | ¹H NMR(300MHz, CDCl₃) δ 0.89(3H, t, 7.43Hz), 1.36(3H, t, 7.12Hz), 1.63(2H, qt, 7.39Hz, 7.43Hz), 2.68(2H, t, 7.39Hz), 3.74(3H, s), |
| | 4.35(2H, q, 7.12Hz), 4.64(2H, d, 7.38Hz), 5.1(1H, m), 6.57(1H, s), 6.82(2H, d, 8.71Hz), 7.23(2H, d, 8.71Hz) |
| 79 | ¹H NMR(500MHz, CDCl₃) δ 1.01(3H, t, 7.34Hz), 1.75(2H, tq, 7.34Hz, 8.01Hz), 2.83(2H, t, 8.01Hz), 3.01(2H, t, 6.87Hz), 3.89(2H, td, |
| | 6.87Hz, 5.92Hz), 5.04(1H, s), 6.66(1H, s), 7.26(3H, m), 7.34(2H, m), 8.48(1H, s) |
| 80 | ¹H NMR(300MHz, CDCl₃) δ 0.91(3H, t, 7.31Hz), 1.36(3H, t, 7.04Hz), 1.62(2H, qt, 7.31Hz, 7.55Hz), 2.68(2H, t, 7.55Hz), 2.91(2H, t, |
| | 6.81Hz), 3.79(2H, td, 6.56Hz, 6.81Hz), 4.35(2H, q, 7.04Hz), 4.92(1H, m), 6.46(1H, s), 7.14-7.29(5H, m) |
| 82 | ¹H NMR(300MHz, CDCl₃) δ 0.99(3H, t, 7.29Hz), 1.73(2H, tq, 7.29Hz, 7.43Hz), 2.82(2H, t, 7.43Hz), 2.91(2H, t, 6.78Hz), 3.82(2H, td, 5.89Hz, |
| | 6.78Hz), 5.02(1H, t, 5.89Hz), 5.44(1H, s), 6.65(1H, s), 6.81(2H, d, 8.45Hz), 7.1(2H, d, 8.45Hz), 8.46(1H, s) |
| 85 | ¹H NMR(500MHz, CDCl₃) δ 1.01(3H, t, 7.35Hz), 1.75(2H, tq, 7.35Hz, 8.02Hz), 2.83(2H, t, 8.02Hz), 2.94(2H, t, 6.84Hz), 3.82(3H, s), |
| | 3.85(2H, td, 6.84Hz, 5.88Hz), 5.03(1H, s), 6.66(1H, s), 6.87(2H, d, 8.64Hz), 7.16(2H, d, 8.64Hz), 8.47(1H, s) |
| 87 | ¹H NMR(300MHz, CDCl₃) δ 0.99(3H, t, 7.29Hz), 1.73(2H, tq, 7.29Hz, 7.43Hz), 2.82(2H, t, 7.43Hz), 2.93(2H, t, 6.79Hz), 3.82(3H, s), 3.85(2H, |
| | td, 6.79Hz, 5.89Hz), 3.88(3H, s), 5.01(1H, t, 5.89Hz), 6.64(1H, s), 6.72-6.85(3H, m), 8.47(1H, s) |
| 90 | ¹H NMR(300MHz, CDCl₃) δ 0.92(3H, t, 7.31Hz), 1.79(2H, m), 2.94(2H, t, 6.79Hz), 3.83(2H, td, 6.4Hz, 6.79Hz), 4.79(2H, t, 6.53Hz), 5.36(1H, |
| | s), 6.83(1H, s), 7.16-7.30(5H, m), 8.41(1H, s) |
| 91 | ¹H NMR(500MHz, CDCl₃) δ 1.38(6H, d, 6.85Hz), 3.21(1H, hd, 0.99Hz, 6.85Hz), 3.81(3H, s), 4.81(2H, d, 5.46Hz), 5.21(1H, s), 6.78(1H, d, |
| | 0.99Hz), 6.86(1H, d, 8.21Hz), 6.95(1H, s), 6.98(1H, d, 7.55Hz), 7.28(1H, dd, 7.55Hz, 8.21Hz), 8.50(1H, s) |
| 92 | ¹H NMR(500MHz, CDCl₃) δ 1.37(6H, d, 6.85Hz), 3.01(2H, t, 6.9Hz), 3.2(1H, hd, 0.99Hz, 6.85Hz), 3.85(2H, td, 5.9Hz, 6.9Hz), 5.05(1H, s), |
| | 6.67(1H, d, 0.99Hz), 7.28(3H, m), 7.34(2H, m), 8.48(1H, s) |
| 93 | ¹H NMR(500MHz, CDCl₃) δ 1.37(6H, d, 6.85Hz), 2.95(2H, t, 6.87Hz), 3.2(1H, hd, 0.96Hz, 6.85Hz), 3.82(3H, s), 3.85(2H, td, 5.86Hz, 6.87Hz), |
| | 5.31(1H, s), 6.67(1H, d, 0.96Hz), 6.88(2H, d, 8.64Hz), 7.17(2H, d, 8.64Hz), 8.48(1H, s) |
| 94 | ¹H NMR(300MHz, CDCl₃) δ 0.94(3H, t, 7.43Hz), 1.4(2H, qt, 7.43Hz, 7.43Hz), 1.69(2H, tt, 7.43Hz, 7.43Hz), 1.85(2H, t, 7.43Hz), 2.99(2H, t, |
| | 6.79Hz), 3.87(2H, td, 5.89Hz, 6.79Hz), 5.03(1H, s), 6.64(1H, s), 7.23-7.36(5H, m), 8.47(1H, s) |
| 95 | ¹H NMR(300MHz, CDCl₃) δ 0.88(6H, d, 6.66Hz), 1.86(1H, m), 2.63(2H, d, 7.17Hz), 2.95(2H, t, 7.04Hz), 3.83(2H, td, 6.32Hz, 7.04Hz), |
| | 5.91(1H, s), 6.74(1H, s), 7.17-7.28(5H, m), 8.41(1H, s) |
| 96 | ¹H NMR(300MHz, CDCl₃) δ 1.35(3H, td, 7.43Hz, 1.02Hz), 2.89(2H, q, 7.43Hz), 3.01(2H, t, 7.04Hz), 3.56(2H, td, 5.89Hz, 7.04Hz), 4.51(1H, |
| | s), 6.42(1H, d, 5.38Hz), 6.68(1H, d, 1.02Hz), 7.22-7.37(5H, m), 8.2(1H, d, 5.38Hz) |

Pharmacology

The compounds provided in the present invention are positive allosteric modulators of mGluR2. As such, these compounds do not appear to bind to the orthosteric glutamate recognition site, and do not activate the mGluR2 by themselves. Instead, the response of mGluR2 to a concentration of glutamate or to an mGluR2 agonist is increased when compounds of Formula (I) are present. Compounds of Formula (I) are expected to have their effect at mGluR2 by virtue of their ability to enhance the function of the receptor upon glutamate or an mGluR2 agonist activation. The behavior of positive allosteric modulators, such as the ones described in Formula I, at mGluR2 is shown in Example A, which is suitable for the identification of such compounds.

### Example A

### [³⁵S]GTPγS Binding Assay

The [³⁵S]GTPγS binding is a functional membrane-based assay used to study G-protein coupled receptor (GPCR) function. This method is using a binding assay to assess the initial step in receptor-mediated G protein activation in membranes prepared from cells expressing recombinant GPCR or using membranes from discrete area of the rat brain. In brief, the assay is measuring the activation of G proteins by catalyzing the exchange of guanosine 5′-diphosphate (GDP) by guanosine 5′-triphosphate (GTP) at the α subunit. The GTP-bounded G proteins dissociate into two subunits, Gα-GTP and Gβγ, which in turn regulate intracellular enzymes and ion channels. GTP is rapidly hydrolysed by the Gα-subunit (GTPases) and the G protein is deactivated and ready for new GTP exchange cycle (Harper (1998) Curr Protoc Pharmacol 2.6.1-10, John Wiley & Sons, Inc.). [³⁵S]GTPγS, a non-hydrolyzed analogue of GTP, is used for this purpose.

This method is widely used to study receptor activation of G protein in membranes prepared from rat brain tissue, including mGluR2 receptors (Schaffhauser et al 2003, Pinkerton et al, 2004). mGluR2 receptors are expressed in the rat brain cortex (Mutel et al (1998) J. Neurochem. 71:2558-64; Schaffhauser et al (1998) Mol. Pharmacol. 53:228-33) and are coupled to Gαi-protein, a preferential coupling for this method. The study of the pharmacological characterisation of metabotropic glutamate receptor-mediated high-affinity GTPase activity (Nishi et al (2000) Br. J. Pharmacol. 130:1664-1670) showed that the activation of G-proteins in rat cerebral cortical membranes is mediated by group II mGluRs, and in particular by mGluR2.

[³⁵S]GTPγS binding assay using cortical rat brain membranes preparation was used and adapted from Schaffhauser et al ((2003) Mol. Pharmacol. 4:798-810) for the detection of the positive allosteric modulator properties of the compounds of this invention on native rat mGluR2. In order to eliminate the possible interference with group III Gαi-protein coupled mGluRs (mGluR4, mGluR7, mGluR8; mGluR6 is not expressed in the cortex (Laurie et al (1997) Neuropharmacol. 36:145-52)), the potentiation of the response to a selective mGluR2 agonist, such as DCG-IV (Cartmell et al. (1998) Br. J. Pharmacol. 123(3):497-504) or LY379268 (Monn et al. (1999) J. Med. Chem 42:1027-40), by compounds described in the present invention was performed.

Membrane preparation. Cortices were dissected out from brains of 200-300 g Sprague-Dawley rats (Charles River Laboratories, L'Arbresle, France). Tissues were homogenized in 6 volumes (vol/wt) of 10% sucrose at 4° C. using a glass-teflon homogenizer. The homogenate was centrifuged at 1250 g for 10 min, and the supernatant was centrifuged at 40,000 g for 20 min (4° C.). The pellet was resuspended in 25 ml water using a Polytron disrupter (Kinematica AG, Luzern, Switzerland) and centrifuged for 10 min at 3000 g. (4° C.). The supernatant was centrifuged at 40,000 g for 20 min (4° C.). The supernatant was discarded and the pellet washed twice by resuspension in 10 volumes 5 mM HEPES-KOH, pH 7.4. The homogenate was frozen and thawed twice and centrifuged at 40,000 g for 20 min. The final pellet was resuspended in 5 mM HEPES-KOH, pH 7.4 and stored at −80° C. before its use. Protein concentration was determined by the Bradford method (Bio-Rad protein assay, Reinach, Switzerland) with bovine serum albumin as standard.

[³⁵S]GTPγS binding assay. Measurement of mGluR2 positive allosteric modulators properties in rat cortical membranes was performed as follows: rat cortical membrane (1.5 μg) were incubated in 96-well microplates for 15 min at 30° C. in assay buffer (50 mM HEPES pH 7.4, 100 mM NaCl, 5 mM MgCl₂, 10 μM GDP, 10 μg/ml saponin, EGTA 0.2 mM) with increasing concentrations of positive allosteric modulator (from 1 nM to 10 μM) and a minimal concentration of DCG-IV or LY379268, a selective mGluR2 agonist, that has been determined in previous experiments and that corresponds to the EC₂₀, a concentration that gives 20% of the maximal response of the agonist, and is in accordance to published data (Pin et al. (1999) Eur. J. Pharmacol. 375:277-294). Likewise, 10-point concentration-response curves of an mGluR2 selective agonist such as DCG-IV or LY379268, were tested in the absence or in the presence of 3 or 10 μM of positive allosteric modulator in order to detect a leftward-shift of the concentration-response curve of the agonist (appreciated by a decrease in the EC₅₀) and/or an increase of its maximal efficacy. After addition of 0.1 nM [³⁵S]GTPγS to achieve a total reaction volume of 200 μl, microplates were shaken for 1 min and further incubated at 30° C. for 30 min. The incubation was stopped by rapid vacuum filtration over glass-fiber filter plates (Unifilter 96-well GF/C filter plates, Perkin-Elmer, Schwerzenbach, Switzerland) microplate using a 96-well plate cell harvester (Filtermate, Perkin-Elmer, Downers Grove, USA). The Unifilter plate was washed three times with 300 g1 of ice-cold wash buffer (20 mM HEPES pH 7.4, 100 mM NaCl). When filters are dried, 40 g1 of liquid scintillation cocktail (Microscint 20) was added to each well. The amount of membrane-bound [³⁵S]GTPγS is measured using a 96-well plate reader (Top-Count, Perkin-Ehmer, Downers Grove, USA). Non specific [³⁵S]GTPγS binding is determined in the presence of 10 μM of GTP.

Data analysis. The concentration-response curves of representative compounds of the present invention in the presence of EC₂₀ of mGluR2 agonist were generated using the Prism Graph-Pad program (Graph Pad Software Inc, San Diego, USA). The curves were fitted to a four-parameter logistic equation (Y=Bottom+(Top-Bottom)/(1+10ˆ((LogEC₅₀—X)*Hill Slope) allowing determination of EC₅₀ values. Each curve was performed using triplicate sample per data point and 10 concentrations. The concentration-response curves of a selective mGluR2 agonist in the absence or in the presence of representative compounds of the present invention were also generated using Prism Graph-Pad program (Graph Pad Software Inc, San Diego, USA). The curves were fitted to a four-parameter logistic equation (Y=Bottom+(Top-Bottom)/(1+10ˆ((LogEC₅₀—X)*Hill Slope) allowing determination of EC₅₀ values of the selective mGluR2 agonist. Each curve was performed using triplicate sample per data point and 10 concentrations.

Data presented in the Figure B below represent the ability of 10 μM of the Compound 28 to increase the [GTPi⁵S] binding induced by 50 nM of DCG-IV, an mGluR2 agonist. Said example has no statistically significant agonistic activity when tested in the absence of 50 nM DCG-IV, as compared to buffer value (0% of maximal response). Instead, when compounds are added together with an mGluR2 agonist like glutamate or DCG-IV, the effect measured is significantly potentiated compared to the effect of the agonist alone at the same concentration. Each bar graph is the mean and S.E.M. of triplicate data points and is representative of three independent experiments.

[image]

Table 5 shows representative compounds of the present invention that were clustered into three classes according to their ability to leftward-shift the concentration-response curve of a selective mGluR2 agonist such as LY379268 and/or to increase its maximal efficacy.

TABLE 5
Summary of activity-data

| Comp. Nr | Activity |
|---|---|
| 5 | + |
| 49 | + |
| 61 | ++ |
| 75 | ++ |
| 42 | ++ |
| 43 | ++ |
| 58 | ++ |
| 79 | +++ |
| 82 | +++ |
| 96 | +++ |
| 15 | +++ |
| 46 | +++ |

(+): left-ward shift of agonist mGluR2 concentration-response curve [<2-fold]
(++): left-ward shift of agonist mGluR2 concentration-response curve [2- to 3.5-fold]
(+++): left-ward shift of agonist mGluR2 concentration-response curve [>3.5-fold]

Thus, the positive allosteric modulators provided in the present invention are expected to increase the effectiveness of glutamate or mGluR2 agonists at mGluR2, and therefore, these positive allosteric modulators are expected to be useful for treatment of various neurological and psychiatric disorders associated with glutamate dysfunction described to be treated herein and others that can be treated by such positive allosteric modulators.

### Formulation Examples

Typical examples of recipes for the Formulation of the invention are as follows:

1. Tablets

| | | |
|---|---|---|
| Compound 28 | 5 to 50 | mg |
| Di-calcium phosphate | 20 | mg |
| Lactose | 30 | mg |
| Talcum | 10 | mg |
| Magnesium stearate | 5 | mg |
| Potato starch | ad 200 | mg |

In this example, Compound 28 can be replaced by the same amount of any of the compounds according to the invention, in particular by the same amount of any of the exemplified compounds.

2. Suspension:

An aqueous suspension is prepared for oral administration so that each 1 milliliter contains 1 to 5 mg of one of the described example, 50 mg of sodium carboxymethyl cellulose, 1 mg of sodium benzoate, 500 mg of sorbitol and water ad 1 ml.

3. Injectable

A parenteral composition is prepared by stirring 1.5% by weight of active ingredient of the invention in 10% by volume propylene glycol and water.

4 Ointment

| | | |
|---|---|---|
| Compound 28 | 5 to 1000 | mg |
| Stearyl alcohol | 3 | g |
| Lanoline | 5 | g |
| White petroleum | 15 | g |
| Water | ad 100 | g |

In this example, Compound 28 can be replaced by the same amount of any of the compounds according to the invention, in particular by the same amount of any of the exemplified compounds.

Reasonable variations are not to be regarded as a departure from the scope of the invention. It will be obvious that the thus described invention may be varied in many ways by those skilled in the art.

1-32. (canceled)

33. A compound of Formula (I) 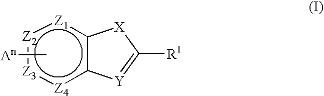
a pharmaceutically acceptable acid or base addition salt thereof, a stereochemically isomeric form thereof and an N-oxide form thereof, wherein
Y is selected from —N— and —C(R²)—;
X is selected from —S—, —S(O)—, —S(O)₂—, —O— and —N(R³)—;
R¹, R² and R³ are each independently selected from the group of hydrogen, halo, —CN, —OH, —NO2, —CF₃, —NH₂, —SH, —C(═NR⁴)NR⁵R⁶, —C(═O)R⁴, —C(═NR⁴)R⁵, —C(═O)O R⁴, —C(═O)NR⁴R⁵, —SR⁴, —S(O)R⁴, —S(O)₂R⁴, —NR⁴R⁵, —NR⁴C(═O)R⁵, —NR⁴C(═NR⁵) R⁶, —NR⁴C(═NR⁵)NR⁶R⁷, —NR⁴C(═O)O R⁵, —NR⁴C(═O)NR⁵R⁶—NR⁴S(O)₂R⁵—S(O)₂NR⁴R⁵, —C(═S)NR⁴R⁵, —OC(═O)R⁴, —OC(═O)NR⁴R⁵, —OR⁴, an optionally substituted radical selected from the group of —(C₁-C₆)alkyl, —(C₁-C₆)alkylhalo, —(C₂-C₆)alkynyl, —(C₂-C₆)alkenyl, —(C₃-C₇)cycloalkyl, —(C₃-C₈)cycloalkenyl, —(C₁-C₆)alkylcyano, —(C₁-C₆)alkylaryl, —(C₁-C₆)alkylheteroaryl, aryl and heteroaryl, and a radical described as —V₁-T₁-M₁;
Z₁, Z₂, Z₃ and Z₄ are each independently selected from a covalent bond, C, S, N and O, with the provision that a 5 or 6 membered heteroaryl or aryl ring is formed, which may optionally be substituted by 1 to 4 radicals Aⁿ;
Aⁿ radicals are each independently selected from the group of hydrogen, halo, —CN, —OH, —NO₂, —CF₃, —SH, —NH₂, an optionally substituted radical selected from the group of —(C₁-C₆)alkyl, —(C₁-C₆)alkylhalo, —(C₂-C₆)alkynyl, —(C₂-C₆)alkenyl, —(C₃-C₇)cycloalkyl, —(C₁-C₆)alkylcyano, —O—(C₁-C₆)alkyl, —O—(C₁-C₆)alkylhalo, —O—(C₁-C₆)alkylcyano, —O—(C₃-C₆)alkynyl, —O—(C₃-C₇)cycloalkyl, —O—(C₂-C₆)alkenyl, —O—(C₂-C₆)alkyl-OR³—O—(C₁-C₆)alkyl-heteroaryl, —O—(C₀-C₆)alkylaryl, —(C₀-C₆)alkyl-OR³, —(C₃-C₇)cycloalkyl-(C₁-C₆)alkyl, —O—(C₃-C₇)cycloalkyl-(C₁-C₆)alkyl, —O-heteroaryl, heteroaryl, —(C₁-C₆)alkyl-heteroaryl, aryl, —O-aryl, —(C₁-C₆)alkylaryl, —(C₁-C₆)alkylhalo-OR⁸, —(C₃-C₆)alkynyl-OR³, —(C₃-C₆)alkenyl-OR³, —(C₀-C₆)alkyl-SR³, —O—(C₂-C₆)alkyl-SR⁸, —(C₁-C₆)alkyl-S(═O)—R⁸, —O—(C₁-C₆)alkyl-S(═O)—R³, —(C₀-C₆)alkyl-S(═O)₂—R³, O—(C₁-C₆)alkyl-S(═O)₂—R³, —(C₀-C₆)alkyl-NR⁸R⁹, —O—(C₂-C₆)alkyl-NR⁸R⁹, —(C₀-C₆)alkyl-S(═O)₂NR⁸R⁹, —(C₀-C₆)alkyl-NR³—S(═O)₂R⁹, —O—(C₁-C₆)alkyl-S(═O)₂NR⁸R⁹, —O—(C₁-C₆)alkyl-NR⁸—S(═O)₂R⁹, —(C₀-C₆)alkyl-C(═O)—NR⁸R⁹, —(C₀-C₆)alkyl-NR⁸C(═O)—R⁹, —O—(C₁-C₆)alkyl-C(═O)—NR⁸R⁹, —O—(C₁-C₆)alkyl-NR⁸C(═O)—R⁹, —(C₀-C₆)alkyl-OC(═O)—R³⁸, —(C₀-C₆)alkyl-C(═O)—OR³, —O—(C₁-C₆)alkyl-OC(═O)—R³, —O—(C₁-C₆)alkyl-C(═O)—OR³, —(C₀-C₆)alkyl-C(═O)—R³, —O—(C₁-C₆)alkyl-C(═O)—R³, —(C₀-C₆)alkyl-NR⁸—C(═O)—OR⁹, —(C₀-C₆)alkyl-O—C(═O)—NR⁸R⁹, —(C₀-C₆)alkyl-NR⁸—C(═NR⁹)—NR¹⁰R¹¹, —(C₀-C₆)alkyl-NR⁸—C(═O)—NR⁹R¹⁰, —(C₀-C₆)alkyl-NR³—C(═S)—NR⁹R¹⁰, and a —V₂-T₂-M₂ radical;
n is an integer ranging from 1 to 4;
T₁, V₁, T₂ and V₂ are each independently selected from the group of a covalent bond, —O—, —C(═O)—, —C(═O)O—, —C(═O)NR¹²—, —S—, —S(O)—, —S(O)₂—, —S(O)₂NR¹², —NR¹², NR¹²C(═O)—, —NR¹²C(═O)NR¹³—, —NR¹²S(O)₂—, —NR¹²C(═S)NR¹³—, —OC(═O)—, —OC(═O)NR¹², —NR¹²C(═O)O—, and an optionally substituted radical selected from the group of —(C₁-C₆)alkyl-, —(C₂-C₆)alkynyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₁-C₆)alkyl-, —(C₀-C₆)alkyl-O—(C₂-C₆)alkynyl-, —(C₀-C₆)alkyl-O—(C₂-C₆)alkenyl-, —(C₀-C₆)alkyl-O—(C₃-C₇)cycloalkyl-, —(C₀-C₆)alkyl-O—(C₄-C₁₀)alkylcycloalkyl-, —(C₀-C₆)alkyl-C(═O)—(C₁-C₆)alkyl-, —(C₀-C₆)alkyl-C(═O)—(C₂-C₆)alkynyl-, —(C₀-C₆)alkyl-C(═O)—(C₂-C₆)alkenyl-, (C₀-C₆)alkyl-C(═O)—(C₄-C₁₀)alkylcycloalkyl-, —(C₀-C₆)alkyl-C(═O)—(C₃-C₇)cycloalkyl-, —(C₀-C₆)alkyl-C(═O)O—(C₁-C₆)alkyl-, —(C₀-C₆)alkyl-C(═O)O—(C₂-C₆)alkynyl-, —(C₀-C₆)alkyl-C(═O)O—(C₂-C₆)alkenyl-, —(C₀-C₆)alkyl-C(═O)O—(C₃-C₇)cycloalkyl-, —(C₀-C₆)alkyl-C(═O)O—(C₄-C₁₀)alkylcycloalkyl-, —(C₀-C₆)alkyl-C(═O)NR¹²—(C₁-C₆)alkyl-, —(C₀-C₆)alkyl-C(═O)NR¹²—(C₂-C₆)alkynyl-, —(C₀-C₆)alkyl-C(═O)NR¹²—(C₂-C₆)alkenyl-, —(C₀-C₆)alkyl-C(═O)NR¹²—(C₃-C₇)cycloalkyl-, —(C₀-C₆)alkyl-C(═O)NR¹² (C₄-C₁₀)alkylcycloalkyl-, —(C₀-C₆)alkyl-S—(C₁-C₆)alkyl-, —(C₀-C₆)alkyl-S—(C₂-C₆)alkynyl-, —(C₀-C₆)alkyl-S—(C₂-C₆)alkenyl-, —(C₀-C₆)alkyl-S—(C₃-C₇)cycloalkyl-, —(C₀-C₆)alkyl-S—(C₄-C₁₀)alkylcycloalkyl-, —(C₀-C₆)alkyl-S(O)—(C₁-C₆)alkyl-, —(C₀-C₆)alkyl-O—(C₂-C₆)alkynyl-, —(C₀-C₆)alkyl-S(O)—(C₂-C₆)alkenyl-, —(C₀-C₆)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₁-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-S(O)₂NR¹²—(C₁-C₆)alkyl-, —(C₀-C₆)alkyl-S(O)₂NR¹²—(C₂-C₆)alkynyl-, —(C₀-C₆)alkyl-S(O)₂NR¹²—(C₂-C₆)alkenyl-, —(C₀-C₆)alkyl-S(O)₂NR¹²—(C₃-C₇)cycloalkyl-, —(C₀-C₆)alkyl-S(O)₂NR¹²—(C₄-C₁₀)alkylcycloalkyl-, —(C₀-C₆)alkyl-NR¹²—(C₁-C₆)alkyl-, —(C₀-C₆)alkyl-NR¹²—(C₂-C₆)alkynyl-, —(C₀-C₆)alkyl-NR¹²—(C₂-C₆)alkenyl-, —(C₀-C₆)alkyl-NR¹² (C₃-C₇)cycloalkyl-, —(C₀-C₆)alkyl-NR¹²—(C₄-C₁₀)alkylcycloalkyl-, —(C₀-C₆)alkyl-NR¹²C(═O)—(C₁-C₆)alkyl-, —(C₀-C₆)alkyl-NR¹²C(═O)—(C₂-C₆)alkynyl-, —(C₀-C₆)alkyl-NR¹²C(═O)—(C₂-C₆)alkenyl-, —(C₀-C₆)alkyl-NR¹²C(═O)—(C₃-C₇)cycloalkyl-, —(C₀-C₆)alkyl-NR¹²C(═O)—(C₄-C₁₀)alkylcycloalkyl-, —(C₀-C₆)alkyl-NR¹²C(═O)NR¹³—(C₁-C₆)alkyl-, —(C₀-C₆)alkyl-NR¹²C(═O)NR¹³—(C₂-C₆)alkynyl-, —(C₀-C₆)alkyl-NR¹²C(═O)NR¹³—(C₂-C₆)alkenyl-, —(C₀-C₆)alkyl-NR²C(═O)NR¹³—(C₃-C₇)cycloalkyl-, —(C₀-C₆)alkyl-NR¹²C(═O)NR¹³—(C₄-C₁₀)alkylcycloalkyl-, —(C₀-C₆)alkyl-NR¹²S(O)₂—(C₁-C₆)alkyl-, —(C₀-C₆)alkyl-NR¹²S(O)₂—(C₂-C₆)alkynyl-, —(C₀-C₆)alkyl-NR¹²S(O)₂—(C₂-C₆)alkenyl-, —(C₀-C₆)alkyl-NR¹²S(O)₂—(C₃-C₇)cycloalkyl-, —(C₀-C₆)alkyl-NR¹²S(O)₂—(C₄-C₁₀)alkylcycloalkyl-, —(C₀-C₆)alkyl-NR¹²C(═S)NR¹³—(C₁-C₆)alkyl-, —(C₀-C₆)alkyl-NR¹²C(═S)NR¹³—(C₂-C₆)alkynyl-, —(C₀-C₆)alkyl-NR¹²C(═S)NR¹³—(C₂-C₆)alkenyl-, —(C₀-C₆) alkyl-NR¹²C(═S)NR¹³—(C₃-C₇)cycloalkyl-, —(C₀-C₆)alkyl-NR¹²C(═S)NR¹³—(C₄-C₁₀)alkylcycloalkyl-, —(C₀-C₆)alkyl-OC(═O)—(C₁-C₆)alkyl-, —(C₀-C₆)alkyl-OC(═O)—(C₂-C₆)alkynyl-, —(C₀-C₆)alkyl-OC(═O)—(C₂-C₆)alkenyl-, —(C₀-C₆)alkyl-OC(═O)—(C₄-C₁₀)alkylcycloalkyl-, —(C₀-C₆)alkyl-OC(═O)—(C₃-C₇)cycloalkyl-, —(C₀-C₆)alkyl-OC(═O)NR¹²—(C₁-C₆)alkyl-, —(C₀-C₆)alkyl-OC(═O)NR¹²—(C₂-C₆)alkynyl-, —(C₀-C₆)alkyl-OC(═O)NR¹²—(C₂-C₆)alkenyl-, —(C₀-C₆)alkyl-OC(═O)NR¹²—(C₄-C₁₀)alkylcycloalkyl-, —(C₀-C₆)alkyl-OC(═O)NR¹²—(C₃-C₇)cycloalkyl-, —(C₀-C₆)alkyl-NR¹²C(═O)O—(C₁-C₆)alkyl-, —(C₀-C₆)alkyl-NR¹²C(═O)O—(C₂-C₆)alkynyl-, —(C₀-C₆)alkyl-NR¹²C(═O)O—(C₂-C₆)alkenyl-, —(C₀-C₆)alkyl-NR¹²C(═O)O— (C₃-C₇)cycloalkyl-, —(C₀-C₆)alkyl-NR¹²C(═O)O— (C₄-C₁₀)alkylcycloalkyl-, —(C₀-C₆)alkyl-NR¹²C(═NR¹³)NR¹⁴—(C₁-C₆)alkyl-, —(C₀-C₆)alkyl-NR¹²C(═NR¹³)NR¹⁴—(C₂-C₆)alkynyl-, —(C₀-C₆)alkyl-NR¹²C(═NR¹³)NR¹⁴—(C₂-C₆)alkenyl-, —(C₀-C₆)alkyl-NR¹²C(═NR¹³)NR¹⁴—(C₃-C₇)cycloalkyl-, —(C₀-C₆)alkyl-NR¹²C(═NR¹³)NR¹⁴—(C₄-C₁₀)alkylcycloalkyl-, —(C₀-C₆)alkyl-NR¹²C(═NR¹³)—(C₁-C₆)alkyl-, —(C₀-C₆)alkyl-NR¹²C(═NR¹³)—(C₂-C₆)alkynyl-, —(C₀-C₆)alkyl-NR¹²C(═NR¹³)—(C₂-C₆)alkenyl-, —(C₀-C₆)alkyl-NR¹²C(═NR¹³)—(C₃-C₇)cycloalkyl-, —(C₀-C₆)alkyl-NR¹²C(═NR¹³)—(C₄-C₁₀)alkylcycloalkyl-, —(C₀-C₆)alkyl-C(═NR¹²)NR¹³—(C₁-C₆)alkyl-, —(C₀-C₆)alkyl-C(═NR¹²)NR³—(C₂-C₆)alkynyl-, —(C₀-C₆)alkyl-C(═NR¹²)NR¹³—(C₂-C₆)alkenyl-, —(C₀-C₆)alkyl-C(═NR¹²)NR¹³—(C₃-C₇)cycloalkyl- and —(C₀-C₆)alkyl-C(═NR¹²)NR¹³—(C₄-C₁₀)alkylcycloalkyl-;
M₁ and M₂ are each independently selected from the group of hydrogen, —CN, —OH, —NO₂, —CF₃, —NH₂, —SH, —C(═O)R¹⁵, —C(═NR¹⁵)R¹⁶, —C(═O)OR¹—C(═O)NR¹⁵R¹⁶—SR¹⁵, —S(O)R¹⁵, —S(O)₂R¹⁵, —NR¹⁵R¹⁶, —NR¹⁵C(═O)R¹⁶, —NR¹⁵C(═NR¹⁶)R¹⁷, NR¹⁵C(═NR¹⁶)NR¹⁷R¹⁸, —NR¹⁵C(═O)OR¹⁶, —NR¹⁵C(═O)NR¹⁶R¹⁷, —NR¹⁵S(O)₂R¹⁶, —C(═S)NR¹⁵R¹⁶—OC(═O)R¹⁵—OC(═O)NR¹⁵R¹⁶, —OR¹⁵, —S(O)₂NR¹⁵R¹⁶, an optionally substituted radical selected from the group of —(C₁-C₆)alkyl, —(C₂-C₆)alkynyl, —(C₂-C₆)alkenyl, —(C₃-C₇)cycloalkyl and —(C₃-C₈)cycloalkenyl, and an optionally substituted 3 to 10 membered ring selected from the group of aryl, heteroaryl, heterocyclic and cycloalkyl rings;
R⁴, R⁵, R⁶, R⁷, R⁸, R⁹, R¹⁰, R¹, R¹², R¹³, R¹⁴, R¹⁵, R¹⁶, R¹⁷ and R¹⁸ are each independently hydrogen or an optionally substituted radical selected from the group of —(C₁-C₆)alkylhalo, —(C₁-C₆)alkyl, —(C₁-C₆)alkylcyano, —(C₂-C₆)alkynyl, —(C₂-C₆)alkenyl, —(C₃-C₇)cycloalkyl, —(C₄-C₁₀)alkylcycloalkyl, heteroaryl, —(C₁-C₆)alkylheteroaryl, aryl, —(C₁-C₆)alkylaryl, —(C₂-C₆)alkynyl-(C₃-C₇)cycloalkyl, —(C₂-C₆)alkynyl-heteroaryl, —(C₂-C₆)alkynyl-aryl, —(C₂-C₆)alkenyl-(C₃-C₇)cycloalkyl, —(C₂-C₆)alkenyl-heteroaryl and —(C₂-C₆)alkenyl-aryl;
R⁴, R⁵, R⁶ and R⁷ may be taken together to form an optionally substituted 3 to 10 membered non-aromatic heterocyclic ring or an optionally substituted 5 to 10 membered aromatic heterocyclic ring;
R⁸, R⁹, R¹⁰ and R¹¹ may be taken together to form an optionally substituted 3 to 10 membered non-aromatic heterocyclic ring or an optionally substituted 5 to 10 membered aromatic heterocyclic ring;
R¹², R¹³ and R¹⁴ may be taken together to form an optionally substituted 3 to 10 membered non-aromatic heterocyclic ring or an optionally substituted 5 to 10 membered aromatic heterocyclic ring; and
R¹⁵, R¹⁶, R¹⁷ and R¹⁸ may be taken together to form an optionally substituted 3 to 10 membered non-aromatic heterocyclic ring or an optionally substituted 5 to 10 membered aromatic heterocyclic ring.

34. A compound according to claim 33 having the Formula (II) 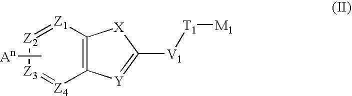
a pharmaceutically acceptable acid or base addition salt thereof, a stereochemically isomeric form thereof and an N-oxide form thereof, wherein:
Z¹, Z², Z³ and Z⁴ are each independently selected from C and N, with the provision that a 5 or 6 membered heteroaryl or aryl ring is formed, which may optionally be substituted by 1 to 4 radicals Aⁿ;
the radical 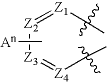
is selected from the group of radicals (a-1), (a-2), (a-3), (a-4), (a-5), (a-6) and (a-7); and 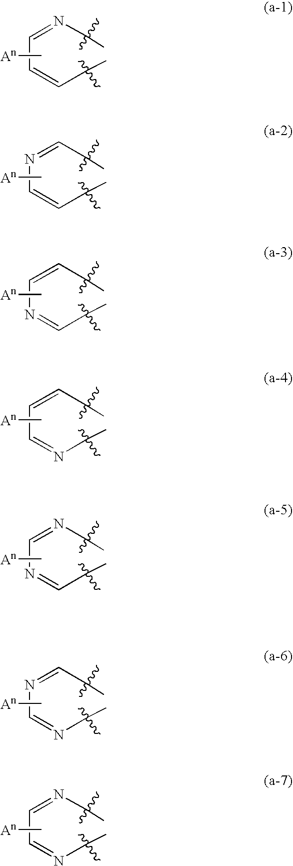
the radical 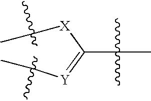
is selected from the group of radicals (b-1), (b-2), (b-3), (b-4), (b-5) and (b-6).

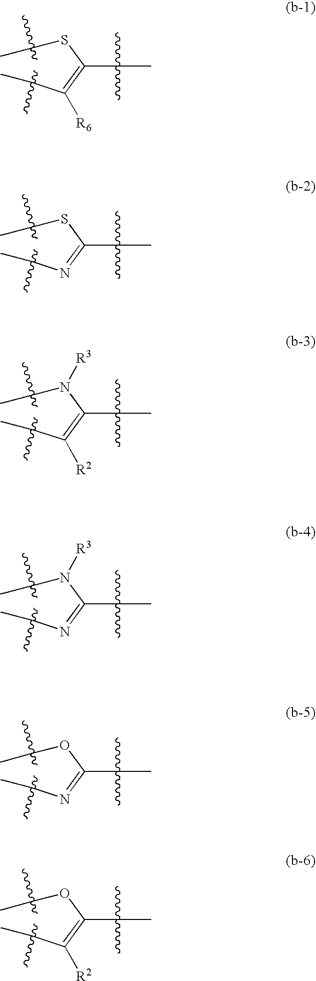

35. A compound according to claim 34 having the Formula (II-a) 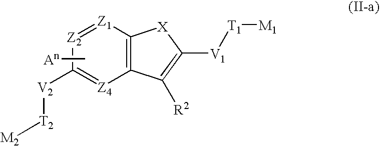
a pharmaceutically acceptable acid or base addition salt thereof, a stereochemically isomeric form thereof and an N-oxide form thereof, wherein:
R² is selected from the group of hydrogen, halo, —CN, —OH, —NO₂, —CF₃, —NH₂, —SH, —C(═NR⁴)NR⁵R⁶, —C(═O)R⁴, —C(═NR⁴)R⁵, —C(═O)OR⁴, —C(═O)NR⁴R⁵, —SR⁴, —S(O)R⁴, —S(O)₂R⁴, —NR⁴R⁵, —NR⁴C(═O)R⁵, —NR⁴C(═NR⁵)R⁶, —NR⁴C(═NR⁵)NR⁶R⁷, —NR⁴C(═O)OR⁵, —NR⁴C(═O)NR⁵R⁶, —NR⁴S(O)₂R⁵, —S(O)₂NR⁴R⁵, —C(═S)NR⁴R⁵, —OC(═O)R⁴, —OC(═O)NR⁴R⁵, —OR⁴, and an optionally substituted radical selected from the group of —(C₁-C₆)alkyl, —(C₁-C₆)alkylhalo, —(C₂-C₆)alkynyl, —(C₂-C₆)alkenyl, —(C₃-C₇)cycloalkyl, —(C₃-C₈)cycloalkenyl, —(C₁-C₆)alkylcyano, —(C₁-C₆)alkylaryl, —(C₁-C₆)alkylheteroaryl, aryl and heteroaryl;
Aⁿ radicals are each independently selected from the group of hydrogen, halo, —CN, —OH, —NO₂, —CF₃, —SH, —NH₂ and an optionally substituted radical selected from the group of —(C₁-C₆)alkyl, —(C₁-C₆)alkylhalo, —(C₂-C₆)alkynyl, —(C₂-C₆)alkenyl, —(C₃-C₇)cycloalkyl, —(C₁-C₆)alkylcyano, —O—(C₁-C₆)alkyl, —O—(C₁-C₆)alkylhalo, —O—(C₁-C₆)alkylcyano, —O— (C₃-C₆)alkynyl, —O— (C₃-C₇)cycloalkyl, —O—(C₂-C₆)alkenyl, —O—(C₂-C₆)alkyl-OR³⁸—O—(C₁-C₆)alkyl-heteroaryl, —O—(C₀-C₆)alkylaryl, —(C₀-C₆)alkyl-OR¹⁸—(C₃-C₇)cycloalkyl-(C₁-C₆)alkyl, —O—(C₃-C₇)cycloalkyl-(C₁-C₆)alkyl, —O— heteroaryl, heteroaryl, —(C₁-C₆)alkyl-heteroaryl, aryl, —O-aryl, —(C₁-C₆)alkylaryl, —(C₁-C₆)alkylhalo-OR³, —(C₃-C₆)alkynyl-OR³, —(C₃-C₆)alkenyl-OR⁸—(C₀-C₆)alkyl-SR⁸, —O—(C₂-C₆)alkyl-SR³, —(C₁-C₆)alkyl-S(═O)—R⁸, —O—(C₁-C₆)alkyl-S(═O)—R⁸, —(C₀-C₆)alkyl-S(═O)₂—R⁸, —O—(C₁-C₆)alkyl-S(═O)₂—R⁸, —(C₀-C₆)alkyl-NR⁸R⁹, —O—(C₂-C₆)alkyl-NR³R⁹, —(C₀-C₆)alkyl-S(═O)₂NR⁸R⁹, —(C₀-C₆)alkyl-NR⁸—S(═O)₂R⁹, —O—(C₁-C₆)alkyl-S(═O)₂NR⁸R⁹, —O—(C₁-C₆)alkyl-NR⁸—S(═O)₂R⁹, —(C₀-C₆)alkyl-C(═O)—NR⁸R⁹, —(C₀-C₆)alkyl-NR⁸C(═O)—R⁹, —O—(C₁-C₆)alkyl-C(═O)—NR⁸R⁹, —O—(C₁-C₆)alkyl-NR⁸C(═O)—R⁹, —(C₀-C₆)alkyl-OC(═O)—R⁸, —(C₀-C₆)alkyl-C(═O)—OR³, —O—(C₁-C₆)alkyl-OC(═O)—R³, —O—(C₁-C₆)alkyl-C(═O)—OR⁸, —(C₀-C₆)alkyl-C(═O)—R³, —O—(C₁-C₆)alkyl-C(═O)—R⁸, —(C₀-C₆)alkyl-NR⁸—C(═O)—OR⁹, —(C₀-C₆)alkyl-O—C(═O)—NR⁸R⁹, —(C₀-C₆)alkyl-NR³—C(═NR⁹)—NR¹⁰R¹¹, —(C₀-C₆)alkyl-NR⁸—C(═O)—NR⁹R¹⁰ and —(C₀-C₆)alkyl-NR³—C(═S)—NR⁹R¹⁰; and
n is an integer ranging from 1 to 3.

36. A compound according to claim 35 having the Formula (II-a1) 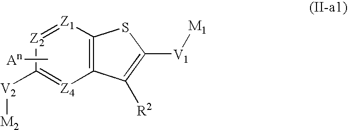
a pharmaceutically acceptable acid or base addition salt thereof, a stereochemically isomeric form thereof and an N-oxide form thereof, wherein:
V₁ and V₂ are each independently selected from the group of a covalent bond, —O—, —C(═O)—, —C(═O)O—, —C(═O)NR¹², —S—, —S(O)—, —S(O)₂—, —S(O)₂NR¹², —NR¹²—, —NR¹²C(═O)—, —NR¹²C(═O)NR¹³—, —NR¹²S(O)₂—, —NR¹²C(═S)NR¹³—, —OC(═O)—, —OC(═O)NR¹², —NR¹²C(═O)O—, and an optionally substituted radical selected from the group of —(C₁-C₆)alkyl-, —(C₂-C₆)alkynyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₁-C₆)alkyl-, —(C₀-C₆)alkyl-O—(C₂-C₆)alkynyl-, —(C₀-C₆)alkyl-O—(C₂-C₆)alkenyl-, —(C₀-C₆)alkyl-O—(C₃-C₇)cycloalkyl-, —(C₀-C₆)alkyl-C(═O)—(C₁-C₆)alkyl-, —(C₀-C₆)alkyl-C(═O)—(C₂-C₆)alkynyl-, —(C₀-C₆)alkyl-C(═O)—(C₂-C₆)alkenyl-, —(C₀-C₆)alkyl-C(═O)—(C₄-C₁₀)alkylcycloalkyl-, —(C₀-C₆)alkyl-C(═O)—(C₃-C₇) cycloalkyl-, —(C₀-C₆)alkyl-C(═O)O—(C₁-C₆)alkyl-, —(C₀-C₆)alkyl-C(═O)O—(C₂-C₆)alkynyl-, —(C₀-C₆)alkyl-C(═O)O—(C₂-C₆)alkenyl-, —(C₀-C₆)alkyl-C(═O)O—(C₃-C₇)cycloalkyl-, —(C₀-C₆)alkyl-C(═O)O—(C₄-C₁₀)alkylcycloalkyl-, —(C₀-C₆)alkyl-C(═O)NR¹²—(C₁-C₆)alkyl-, —(C₀-C₆)alkyl-C(═O)NR¹²—(C₂-C₆)alkynyl-, —(C₀-C₆)alkyl-C(═O)NR¹²—(C₂-C₆)alkenyl-, —(C₀-C₆)alkyl-C(═O)NR¹²—(C₃-C₇)cycloalkyl-, —(C₀-C₆)alkyl-C(═O)NR¹²—(C₄-C₁₀)alkylcycloalkyl-, —(C₀-C₆)alkyl-S—(C₁-C₆)alkyl-, —(C₀-C₆)alkyl-S—(C₂-C₆)alkynyl-, —(C₀-C₆)alkyl-S—(C₂-C₆)alkenyl-, —(C₀-C₆)alkyl-S—(C₃-C₇)cycloalkyl-, —(C₀-C₆)alkyl-S—(C₄-C₁₀)alkylcycloalkyl-, —(C₀-C₆)alkyl-S(O)—(C₁-C₆)alkyl-, —(C₀-C₆)alkyl-O—(C₂-C₆)alkynyl-, —(C₀-C₆)alkyl-S(O)—(C₂-C₆)alkenyl-, —(C₀-C₆)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₁-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-S(O)₂NR¹²—(C₁-C₆)alkyl-, —(C₀-C₆)alkyl-S(O)₂NR¹²—(C₂-C₆)alkynyl-, —(C₀-C₆)alkyl-S(O)₂NR¹²—(C₂-C₆)alkenyl-, —(C₀-C₆)alkyl-S(O)₂NR¹²—(C₃-C₇)cycloalkyl-, —(C₀-C₆)alkyl-S(O)₂NR¹²—(C₄-C₁₀)alkylcycloalkyl-, —(C₀-C₆)alkyl-NR¹²—(C₁-C₆)alkyl-, —(C₀-C₆)alkyl-NR¹²—(C₂-C₆)alkynyl-, —(C₀-C₆)alkyl-NR¹²—(C₂-C₆)alkenyl-, —(C₀-C₆)alkyl-NR¹²—(C₃-C₇)cycloalkyl-, —(C₀-C₆)alkyl-NR¹² (C₄-C₁₀)alkylcycloalkyl-, —(C₀-C₆)alkyl-NR¹²C(═O)—(C₁-C₆)alkyl-, —(C₀-C₆)alkyl-NR¹²C(═O)—(C₂-C₆)alkynyl-, —(C₀-C₆)alkyl-NR¹²C(═O)—(C₂-C₆)alkenyl-, —(C₀-C₆)alkyl-NR¹²C(═O)—(C₃-C₇)cycloalkyl-, —(C₀-C₆)alkyl-NR¹²C(═O)—(C₄-C₁₀)alkylcycloalkyl-, —(C₀-C₆)alkyl-NR¹²C(═O)NR¹³—(C₁-C₆)alkyl-, —(C₀-C₆)alkyl-NR¹²C(═O)NR¹³—(C₂-C₆)alkynyl-, —(C₀-C₆)alkyl-NR¹²C(═O)NR¹³—(C₂-C₆)alkenyl-, —(C₀-C₆)alkyl-NR¹²C(═O)NR¹³—(C₃-C₇)cycloalkyl-, —(C₀-C₆)alkyl-NR¹²C(═O)NR¹³—(C₄-C₁₀)alkylcycloalkyl-, —(C₀-C₆)alkyl-NR¹²S(O)₂—(C₁-C₆)alkyl-, —(C₀-C₆)alkyl-NR¹²S(O)₂—(C₂-C₆)alkynyl-, —(C₀-C₆)alkyl-NR¹²S(O)₂—(C₂-C₆)alkenyl-, —(C₀-C₆)alkyl-NR¹²S(O)₂—(C₃-C₇)cycloalkyl- and —(C₀-C₆)alkyl-NR¹²S(O)₂—(C₄-C₁₀)alkylcycloalkyl-.

37. A compound according to claim 36 wherein:
V₁ is a radical selected from the group of —O—, —C(═O)—, —C(═O)O—, —C(═O)NR¹²—, —S—, —S(O)—, —S(O)₂—, —S(O)₂NR¹²—, —NR¹²—; —NR¹²C(═O)—, —NR¹²C(═O)NR¹³—, —NR¹²S(O)₂—, —NR¹²C(═S)NR¹³—, —OC(═O)—, —OC(═O)NR¹², —NR¹²C(═O)O—, and an optionally substituted radical selected from the group of —(C₁-C₆)alkyl-, —(C₂-C₆)alkynyl-, —(C₂-C₆)alkenyl-, —(C₃-C₇)cycloalkyl-, —(C₁-C₆)alkylhalo-, —(C₁-C₆)alkylcyano-, —(C₀-C₆)alkyl-O—(C₁-C₆)alkyl-, —(C₀-C₆)alkyl-O—(C₃-C₇)cycloalkyl-, —(C₀-C₆)alkyl-C(═O)—(C₁-C₆)alkyl-, —(C₀-C₆)alkyl-C(═O)—(C₄-C₁₀)cycloalkyl-, —(C₀-C₆)alkyl-C(═O)O—(C₁-C₆)alkyl-, —(C₀-C₆)alkyl-C(═O)O—(C₃-C₇)cycloalkyl-, —(C₀-C₆)alkyl-C(═O)NR¹²—(C₁-C₆)alkyl-, —(C₀-C₆)alkyl-C(═O)NR¹²—(C₃-C₇)cycloalkyl-, —(C₀-C₆)alkyl-S—(C₁-C₆)alkyl-, —(C₀-C₆)alkyl-S—(C₃-C₇)cycloalkyl-, —(C₀-C₆)alkyl-S(O)—(C₁-C₆)alkyl-, —(C₀-C₆)alkyl-S(O)—(C₃-C₇)cycloalkyl-, —(C₀-C₆)alkyl-S(O)₂—(C₁-C₆)alkyl-, —(C₀-C₆)alkyl-S(O)₂—(C₃-C₇)cycloalkyl-, —(C₀-C₆)alkyl-S(O)₂NR¹²—(C₁-C₆)alkyl-, —(C₀-C₆)alkyl-S(O)₂NR¹²—(C₃-C₇)cycloalkyl-, —(C₀-C₆)alkyl-NR¹²—(C₁-C₆)alkyl-, —(C₀-C₆)alkyl-NR¹²—(C₃-C₇)cycloalkyl-, —(C₀-C₆)alkyl-NR¹²C(═O)—(C₁-C₆)alkyl-, —(C₀-C₆)alkyl-NR¹²C(═O)—(C₃-C₇)cycloalkyl-, —(C₀-C₆)alkyl-NR¹²C(═O)NR¹³—(C₁-C₆)alkyl-, —(C₀-C₆)alkyl-NR¹²C(═O)NR¹³—(C₃-C₇)cycloalkyl-, —(C₀-C₆)alkyl-NR¹²S(O)₂—(C₁-C₆)alkyl- and —(C₀-C₆)alkyl-NR¹²S(O)₂—(C₃-C₇)cycloalkyl-.

38. A compound according to claim 34 having the Formula (II-b) 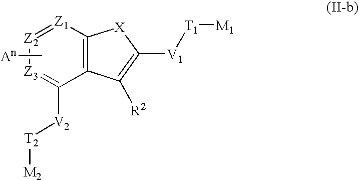
a pharmaceutically acceptable acid or base addition salt thereof, a stereochemically isomeric form thereof and an N-oxide form thereof, wherein:
R² is selected from the group of hydrogen, halo, —CN, —OH, —NO₂, —CF₃, —NH₂, —SH, —C(═NR⁴)NR⁵R⁶, —C(═O)R⁴, —C(═NR⁴)R⁵, —C(═O)OR⁴, —C(═O)NR⁴R⁵, —SR⁴, —S(O)R⁴, —S(O)₂R⁴, —NR⁴R⁵, —NR⁴C(═O)R⁵, —NR⁴C(═NR⁵)R⁶, —NR⁴C(═NR⁵)NR⁶R⁷, —NR⁴C(═O)OR⁵, —NR⁴C(═O)NR⁵R⁶, —NR⁴S(O)₂R⁵, S(O)₂NR⁴R⁵—C(═S)NR⁴R⁵, —OC(═O)R⁴, —OC(═O)NR⁴R⁵, —OR⁴, and an optionally substituted radical selected from the group of —(C₁-C₆)alkyl, —(C₁-C₆)alkylhalo, —(C₂-C₆)alkynyl, —(C₂-C₆)alkenyl, —(C₃-C₇)cycloalkyl, —(C₃-C₈)cycloalkenyl, —(C₁-C₆)alkylcyano, —(C₁-C₆)alkylaryl, —(C₁-C₆)alkylheteroaryl, aryl and heteroaryl;
Aⁿ radicals are each independently selected from the group of hydrogen, halo, —CN, —OH, —NO₂, —CF₃, —SH, —NH₂ and an optionally substituted radical selected from the group of —(C₁-C₆)alkyl, —(C₁-C₆)alkylhalo, —(C₂-C₆)alkynyl, —(C₂-C₆)alkenyl, —(C₃-C₇)cycloalkyl, —(C₁-C₆)alkylcyano, —O—(C₁-C₆)alkyl, —O—(C₁-C₆)alkylhalo, —O—(C₁-C₆)alkylcyano, —O— (C₃-C₆)alkynyl, —O— (C₃-C₇)cycloalkyl, —O—(C₂-C₆)alkenyl, —O—(C₂-C₆)alkyl-OR⁸, —O—(C₁-C₆)alkyl-heteroaryl, —O—(C₀-C₆)alkylaryl, —(C₀-C₆)alkyl-OR³, —(C₃-C₇)cycloalkyl-(C₁-C₆)alkyl, —O—(C₃-C₇)cycloalkyl-(C₁-C₆)alkyl, —O-heteroaryl, heteroaryl, —(C₁-C₆)alkyl-heteroaryl, aryl, —O-aryl, —(C₁-C₆)alkylaryl, —(C₁-C₆)alkylhalo-OR³, —(C₃-C₆)alkynyl-OR³, —(C₃-C₆)alkenyl-OR⁸, —(C₀-C₆)alkyl-SR⁸, —O—(C₂-C₆)alkyl-SR³, —(C₁-C₆)alkyl-S(═O)—R⁸, —O—(C₁-C₆)alkyl-S(═O)—R⁸, —(C₀-C₆)alkyl-S(═O)₂—R⁸, —O—(C₁-C₆)alkyl-S(═O)₂—R⁸, —(C₀-C₆)alkyl-NR⁸R⁹, —O—(C₂-C₆)alkyl-NR⁸R⁹, —(C₀-C₆)alkyl-S(═O)₂NR⁸R⁹, —(C₀-C₆)alkyl-NR⁸—S(═O)₂R⁹, —O—(C₁-C₆)alkyl-S(═O)₂NR⁸R⁹, —O—(C₁-C₆)alkyl-NR⁸—S(═O)₂R⁹, —(C₀-C₆)alkyl-C(═O)—NR⁸R⁹, —(C₀-C₆)alkyl-NR⁸C(═O)—R⁹, —O—(C₁-C₆)alkyl-C(═O)—NR⁸R⁹, —O—(C₁-C₆)alkyl-NR⁸C(═O)—R⁹, —(C₀-C₆)alkyl-OC(═O)—R⁸, —(C₀-C₆)alkyl-C(═O)—OR³, —O—(C₁-C₆)alkyl-OC(═O)—R³, —O—(C₁-C₆)alkyl-C(═O)—OR⁸, —(C₀-C₆)alkyl-C(═O)—R³, —O—(C₁-C₆)alkyl-C(═O)—R⁸, —(C₀-C₆)alkyl-NR⁸—C(═O)—OR⁹, —(C₀-C₆)alkyl-O—C(═O)—NR⁸R⁹, —(C₀-C₆)alkyl-NR³—C(═NR⁹)—NR¹⁰R¹¹, —(C₀-C₆)alkyl-NR⁸—C(═O)—NR⁹R¹⁰ and —(C₀-C₆)alkyl-NR³—C(═S)—NR⁹R¹⁰; and
n is an integer ranging from 1 to 3.

39. A compound according to claim 38 having the Formula (II-b1) 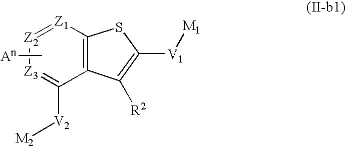
a pharmaceutically acceptable acid or base addition salt thereof, a stereochemically isomeric form thereof and an N-oxide form thereof, wherein:
V₁ and V₂ are each independently selected from the group of a covalent bond, —O—, —C(═O)—, —C(═O)O—, —C(═O)NR¹², —S—, —S(O)—, —S(O)₂—, —S(O)₂NR¹²—, —NR¹²—, —NR¹²C(═O)—, —NR¹²C(═O)NR¹³—, —NR¹²S(O)₂—, —NR¹²C(═S)NR¹³—, —OC(═O)—, —OC(═O)NR¹², —NR¹²C(═O)0, and an optionally substituted radical selected from the group of —(C₁-C₆)alkyl-, —(C₂-C₆)alkynyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₁-C₆)alkyl-, —(C₀-C₆)alkyl-O—(C₂-C₆)alkynyl-, —(C₀-C₆)alkyl-O—(C₂-C₆)alkenyl-, —(C₀-C₆)alkyl-O—(C₃-C₇)cycloalkyl-, —(C₀-C₆)alkyl-C(═O)—(C₁-C₆)alkyl-, —(C₀-C₆)alkyl-C(═O)—(C₂-C₆)alkynyl-, —(C₀-C₆)alkyl-C(═O)—(C₂-C₆)alkenyl-, —(C₀-C₆)alkyl-C(═O)—(C₄-C₁₀)alkylcycloalkyl-, —(C₀-C₆)alkyl-C(═O)—(C₃-C₇)cycloalkyl-, —(C₀-C₆)alkyl-C(═O)O—(C₁-C₆)alkyl-, —(C₀-C₆)alkyl-C(═O)O—(C₂-C₆)alkynyl-, —(C₀-C₆)alkyl-C(═O)O—(C₂-C₆)alkenyl-, —(C₀-C₆)alkyl-C(═O)O—(C₃-C₇)cycloalkyl-, —(C₀-C₆)alkyl-C(═O)O—(C₄-C₁₀)alkylcycloalkyl-, —(C₀-C₆)alkyl-C(═O)NR¹²—(C₁-C₆)alkyl-, —(C₀-C₆)alkyl-C(═O)NR¹²—(C₂-C₆)alkynyl-, —(C₀-C₆)alkyl-C(═O)NR¹²—(C₂-C₆)alkenyl-, —(C₀-C₆)alkyl-C(═O)NR¹²—(C₃-C₇)cycloalkyl-, —(C₀-C₆)alkyl-C(═O)NR¹²—(C₄-C₁₀)alkylcycloalkyl-, —(C₀-C₆)alkyl-S—(C₁-C₆)alkyl-, —(C₀-C₆)alkyl-S—(C₂-C₆)alkynyl-, —(C₀-C₆)alkyl-S—(C₂-C₆)alkenyl-, —(C₀-C₆)alkyl-S—(C₃-C₇)cycloalkyl-, —(C₀-C₆)alkyl-S—(C₄-C₁₀)alkylcycloalkyl-, —(C₀-C₆)alkyl-S(O)—(C₁-C₆)alkyl-, —(C₀-C₆)alkyl-O—(C₂-C₆)alkynyl-, —(C₀-C₆)alkyl-S(O)—(C₂-C₆)alkenyl-, —(C₀-C₆)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₁-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-S(O)₂NR¹²—(C₁-C₆)alkyl-, —(C₀-C₆)alkyl-S(O)₂NR¹²—(C₂-C₆)alkynyl-, —(C₀-C₆)alkyl-S(O)₂NR¹²—(C₂-C₆)alkenyl-, —(C₀-C₆)alkyl-S(O)₂NR¹²—(C₃-C₇)cycloalkyl-, —(C₀-C₆)alkyl-S(O)₂NR¹²—(C₄-C₁₀)alkylcycloalkyl-, —(C₀-C₆)alkyl-NR¹²—(C₁-C₆)alkyl-, —(C₀-C₆)alkyl-NR¹²—(C₂-C₆)alkynyl-, —(C₀-C₆)alkyl-NR¹²—(C₂-C₆)alkenyl-, —(C₀-C₆)alkyl-NR¹²—(C₃-C₇)cycloalkyl-, —(C₀-C₆)alkyl-NR¹² (C₄-C₁₀)alkylcycloalkyl-, —(C₀-C₆)alkyl-NR¹²C(═O)—(C₁-C₆)alkyl-, —(C₀-C₆)alkyl-NR¹²C(═O)—(C₂-C₆)alkynyl-, —(C₀-C₆)alkyl-NR¹²C(═O)—(C₂-C₆)alkenyl-, —(C₀-C₆)alkyl-NR¹²C(═O)—(C₃-C₇)cycloalkyl-, —(C₀-C₆)alkyl-NR¹²C(═O)—(C₄-C₁₀)alkylcycloalkyl-, —(C₀-C₆)alkyl-NR¹²C(═O)NR¹³—(C₁-C₆)alkyl-, —(C₀-C₆)alkyl-NR¹²C(═O)NR¹³—(C₂-C₆)alkynyl-, —(C₀-C₆)alkyl-NR¹²C(═O)NR¹³—(C₂-C₆)alkenyl-, —(C₀-C₆)alkyl-NR¹²C(═O)NR¹³—(C₃-C₇)cycloalkyl-, —(C₀-C₆)alkyl-NR¹²C(═O)NR¹³—(C₄-C₁₀)alkylcycloalkyl-, —(C₀-C₆)alkyl-NR¹²S(O)₂—(C₁-C₆)alkyl-, —(C₀-C₆)alkyl-NR¹²S(O)₂—(C₂-C₆)alkynyl-, —(C₀-C₆)alkyl-NR¹²S(O)₂—(C₂-C₆)alkenyl-, —(C₀-C₆)alkyl-NR¹²S(O)₂—(C₃-C₇)cycloalkyl- and —(C₀-C₆)alkyl-NR¹²S(O)₂—(C₄-C₁₀)alkylcycloalkyl-.

40. A compound according to claim 39, wherein:
V₁ is selected from the group of a covalent bond, —O—, —C(═O)—, —C(═O)O—, —C(═O)NR¹²—, —S—, —S(O)—, —S(O)₂—, —S(O)₂NR¹²—, —NR¹²—, —NR¹²C(═O)—, —NR¹²C(═O)NR¹³—, —NR¹²S(O)₂—, —NR¹²C(═S)NR¹³—, —OC(═O)—, —OC(═O)NR¹², —NR¹²C(═O)O—, and an optionally substituted radical selected from the group of —(C₁-C₆)alkyl-, —(C₂-C₆)alkynyl-, —(C₂-C₆)alkenyl-, —(C₃-C₇)cycloalkyl-, —(C₁-C₆)alkylhalo-, —(C₁-C₆)alkylcyano-, —(C₀-C₆)alkyl-O—(C₁-C₆)alkyl-, —(C₀-C₆)alkyl-O—(C₃-C₇)cycloalkyl-, —(C₀-C₆)alkyl-C(═O)—(C₁-C₆)alkyl-, —(C₀-C₆)alkyl-C(═O)—(C₄-C₁₀)cycloalkyl-, —(C₀-C₆)alkyl-C(═O)O—(C₁-C₆)alkyl-, —(C₀-C₆)alkyl-C(═O)O—(C₃-C₇)cycloalkyl-, —(C₀-C₆)alkyl-C(═O)NR¹²—(C₁-C₆)alkyl-, —(C₀-C₆)alkyl-C(═O)NR¹²—(C₃-C₇)cycloalkyl-, —(C₀-C₆)alkyl-S—(C₁-C₆)alkyl-, —(C₀-C₆)alkyl-S—(C₃-C₇)cycloalkyl-, —(C₀-C₆)alkyl-S(O)—(C₁-C₆)alkyl-, —(C₀-C₆)alkyl-S(O)—(C₃-C₇)cycloalkyl-, —(C₀-C₆)alkyl-S(O)₂—(C₁-C₆)alkyl-, —(C₀-C₆)alkyl-S(O)₂—(C₃-C₇)_{c}-cycloalkyl-, —(C₀-C₆)alkyl-S(O)₂NR¹²—(C₁-C₆)alkyl-, —(C₀-C₆)alkyl-S(O)₂NR¹²—(C₃-C₇)cycloalkyl-, —(C₀-C₆)alkyl-NR¹²—(C₁-C₆)alkyl-, —(C₀-C₆)alkyl-NR¹²—(C₃-C₇)cycloalkyl-, —(C₀-C₆)alkyl-NR¹²C(═O)—(C₁-C₆)alkyl-, —(C₀-C₆)alkyl-NR¹²C(═O)—(C₃-C₇)cycloalkyl-, —(C₀-C₆)alkyl-NR¹²C(═O)NR¹³—(C₁-C₆)alkyl-, —(C₀-C₆)alkyl-NR¹²C(═O)NR¹³—(C₃-C₇)cycloalkyl-, —(C₀-C₆)alkyl-NR¹²S(O)₂—(C₁-C₆)alkyl- and —(C₀-C₆)alkyl-NR¹²S(O)₂—(C₃-C₇)cycloalkyl-; and
M₂ is an optionally substituted 3 to 10 membered ring selected from the group of aryl, heteroaryl, heterocyclic and cycloalkyl rings.

41. A compound according to claim 40 having the Formula (II-b2) 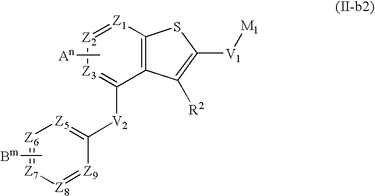
a pharmaceutically acceptable acid or base addition salt thereof, a stereochemically isomeric form thereof and an N-oxide form thereof, wherein:
Z₅, Z₆, Z₇, Z₈ and Z₉ are each independently selected from a covalent bond, C, S, N and O, with the provision that a 5 or 6 membered heteroaryl or aryl ring is formed, which may further be substituted by 1 to 5 radicals B^{m};
B^{m} radicals are each independently selected from the group of hydrogen, halo, —CN, —OH, —NO2, —CF₃, —SH, —NH₂, and an optionally substituted radical selected from the group of —(C₁-C₆)alkyl, —(C₁-C₆)alkylhalo, —(C₂-C₆)alkynyl, —(C₂-C₆)alkenyl, —(C₃-C₇)cycloalkyl, —(C₁-C₆)alkylcyano, —O—(C₁-C₆)alkyl, —O—(C₁-C₆)alkylhalo, —O—(C₁-C₆)alkylcyano, —O— (C₃-C₆)alkynyl, —O— (C₃-C₇)cycloalkyl, —O—(C₂-C₆)alkenyl, —O—(C₂-C₆)alkyl-OR²²—(C₀-C₆)alkyl-OR²², —O-heteroaryl, heteroaryl, —(C₃-C₆)alkynyl-OR²², —(C₃-C₆)alkenyl-OR²², —(C₀-C₆)alkyl-S—R²², —(C₀-C₆)alkyl-NR²²R²³, —O—(C₂-C₆)alkyl-NR²²R²³, —(C₀-C₆)alkyl-S(═O)₂NR²²R²³, —(C₀-C₆)alkyl-NR²²—S(═O)₂R²³, —-(C₁-C₆)alkyl-S(═O)₂NR²²R²³, —O—(C₁-C₆)alkyl-NR²²—S(═O)₂R²³, —(C₀-C₆)alkyl-C(═O)—NR²²R²³, —(C₀-C₆)alkyl-NR²²C(═O)—R²³, —O—(C₁-C₆)alkyl-C(═O)—NR²²R²³, —O—(C₁-C₆)alkyl-NR²²C(═O)—R²³, —(C₀-C₆)alkyl-OC(═O)—R²², —(C₀-C₆)alkyl-C(═O)—OR²², —O—(C₁-C₆)alkyl-OC(═O)—R²², —O—(C₁-C₆)alkyl-C(═O)—OR²², —(C₀-C₆)alkyl-C(═O)—R²² and —O—(C₁-C₆)alkyl-C(═O)—R²²;
m is an integer ranging from 1 to 5;
R²² and R²³ are each independently hydrogen or an optionally substituted radical selected from the group of —(C₁-C₆)alkylhalo, —(C₁-C₆)alkyl, —(C₁-C₆)alkylcyano, —(C₂-C₆)alkynyl, —(C₂-C₆)alkenyl, —(C₃-C₇)cycloalkyl, —(C₄-C₁₀)alkylcycloalkyl, heteroaryl, —(C₁-C₆)alkylheteroaryl, aryl, —(C₁-C₆)alkylaryl, —(C₂-C₆)alkynyl-(C₃-C₇)cycloalkyl, —(C₂-C₆)alkynyl-heteroaryl, —(C₂-C₆)alkynyl-aryl, —(C₂-C₆)alkenyl-(C₃-C₇)cycloalkyl, —(C₂-C₆)alkenyl-heteroaryl and —(C₂-C₆)alkenyl-aryl;
Z₁, Z₂ and Z₃ are each independently selected from C and N, provided that at least 1 nitrogen is present;
V₁ and V₂ are each independently selected from the group of a covalent bond, —C(═O)—, and an optionally substituted radical selected from the group of —(C₁-C₆)alkyl, —(C₂-C₆)alkynyl, —(C₂-C₆)alkenyl, —(C₃-C₇)cycloalkyl, —(C₁-C₆)alkylhalo, —(C₀-C₆)alkyl-C(═O)—(C₀-C₆)alkyl, —(C₀-C₆)alkyl-C(═O)NR⁷—(C₀-C₆)alkyl, —(C₀-C₆)alkyl-O—(C₀-C₆)alkyl, —CO—C₆)alkyl-S—(C₀-C₆)alkyl, —(C₀-C₆)alkyl-S(O)₂—(C₀-C₆)alkyl, —(C₀-C₆)alkyl-S(O)₂NR⁷—(C₀-C₆)alkyl, —(C₀-C₆)alkyl-NR⁷—(C₀-C₆)alkyl, —(C₀-C₆)alkyl-NR⁷C(═O)-(C₀-C₆)alkyl and —(C₀-C₆)alkyl-NR⁷S(O)₂—(C₀-C₆)alkyl;
R⁷ is hydrogen or an optionally substituted radical selected from the group of —(C₁-C₆)alkyl, —(C₁-C₆)alkylhalo, —(C₂-C₆)alkynyl, —(C₂-C₆)alkenyl, —(C₃-C₇)cycloalkyl and —(C₁-C₆)alkylcyano; and
Aⁿ is selected from the group of hydrogen, halo, —CN, —OH, —NO2, —CF₃, —NH₂, and an optionally substituted radical selected from the group of —(C₁-C₆)alkyl, —(C₁-C₆)alkylhalo, —(C₂-C₆)alkynyl, —(C₂-C₆)alkenyl, —(C₃-C₇)cycloalkyl, —(C₁-C₆)alkylcyano, —O—(C₁-C₆)alkyl, —O—(C₁-C₆)alkylhalo, —O—(C₁-C₆)alkylcyano, —O—(C₃-C₆)alkynyl, —O—(C₃-C₇)cycloalkyl, —O—(C₂-C₆)alkenyl, —O—(C₂-C₆)alkyl-OR⁸—(C₀-C₆)alkyl-OR³, —O-heteroaryl, —(C₀-C₆)alkyl-SR⁸, —(C₀-C₆)alkyl-S(═O)₂R⁸, —O—(C₁-C₆)alkyl-S(═O)₂R³, —(C₀-C₆)alkyl-NR⁸R⁹, —(C₀-C₃)alkyl-O—(C₂-C₆)alkyl-NR⁸R⁹, —(C₀-C₆)alkyl-C(═O)—NR⁸R⁹, —(C₀-C₆)alkyl-NR⁸C(═O)—R⁹, —(C₀-C₆)alkyl-C(═O)—R⁸ and —O—(C₁-C₆)alkyl-C(═O)—R⁸.

42. A compound according to claim 41, a pharmaceutically acceptable acid or base addition salt thereof, a stereochemically isomeric form thereof and an N-oxide form thereof, wherein:
Z₁, Z₂, and Z₃ are each independently selected from C and N, provided that at least two nitrogens are present:
V₁ may be selected from the group of a covalent bond, —C(═O)—, and an optionally substituted radical selected from the group of —(C₁-C₆)alkyl-, —(C₀-C₆)alkyl-O—(C₁-C₆)alkyl-, —(C₀-C₆)alkyl-S—(C₁-C₆)alkyl- and —(C₀-C₆)alkyl-NR¹²—(C₁-C₆)alkyl- optionally substituted by one or more radicals from the group of —OCH₃, —OCF₃, CF₃, —F and —CN;
V₂ is an optionally substituted radical selected from the group of —(C₁-C₆)alkyl, —(C₂-C₆)alkynyl, —(C₂-C₆)alkenyl, —(C₃-C₇)cycloalkyl, —(C₁-C₆)alkylhalo, —(C₀-C₆)alkyl-C(═O)—(C₀-C₆)alkyl, —(C₀-C₆)alkyl-C(═O)NR⁷—(C₀-C₆)alkyl, —(C₀-C₆)alkyl-O—(C₀-C₆)alkyl, —(C₀-C₆)alkyl-S—(C₀-C₆)alkyl, —(C₀-C₆)alkyl-S(O)₂—(C₀-C₆)alkyl, —(C₀-C₆)alkyl-S(O)₂NR⁷—(C₀-C₆)alkyl, —(C₀-C₆)alkyl-NR⁷—(C₀-C₆)alkyl, —(C₀-C₆)alkyl-NR⁷C(═O)-(C₀-C₆)alkyl and —(C₀-C₆)alkyl-NR⁷S(O)₂—(C₀-C₆)alkyl;
R² is selected from the group of hydrogen, halo, —OCH₃, —OCF₃, CF₃, and a linear (C₁-C₆)alkyl radical, optionally substituted by —CN, —OCH₃, —OCF₃, CF₃ or halo;
Aⁿ is selected from the group of hydrogen, halo, —CN, —OH, —CF₃, —NH₂, and an optionally substituted radical selected from the group of —(C₁-C₆)alkyl, —(C₁-C₆)alkylhalo, —(C₂-C₆)alkynyl, —(C₂-C₆)alkenyl, —(C₃-C₇)cycloalkyl, —(C₁-C₆)alkylcyano, —O—(C₁-C₆)alkyl, —O—(C₁-C₆)alkylhalo, —O—(C₁-C₆)alkylcyano, —O—(C₃-C₆)alkynyl, —O—(C₃-C₇)cycloalkyl, —O—(C₂-C₆)alkenyl, —O—(C₂-C₆)alkyl-OR¹⁸, —(C₀-C₆)alkyl-OR¹³, —(C₀-C₆)alkyl-NR¹⁸R¹⁹ and —(C₀-C₃)alkyl-O—(C₂-C₆)alkyl-NR¹⁸R¹⁹; and the radical 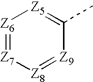
is selected from the group of aryl, thienyl, pyridyl, thiazolyl, isothiazolyl, oxazolyl, isoxazolyl and pyrimidinyl, each radical optionally substituted by m B^{m} radicals.

43. A compound according claim 33 wherein
X is —S—;
R¹ is —(C₁-C₆)alkyl or a radical V₁-T₁-M₁;
Z₁, Z₂, Z₃ and Z₄ are each independently selected from C and N; with the provision that a 6-membered heteroaryl ring is formed, which is substituted with n radicals Aⁿ;
Aⁿ radicals are each independently selected from the group of hydrogen, halo, —(C₁-C₆)alkyl, —O—(C₁-C₆)alkyl, —(C₀-C₆)alkyl-NR⁸R⁹, and a radical V2-T2-M2 n is an integer ranging from 1 to 2;
T₁ and T₂ are each a covalent bond;
V₁ and V₂ are each independently selected from the group of a covalent bond, —C(═O)—, and an optionally substituted radical selected from the group of —(C₁-C₆)alkyl-, —(C₀-C₆)alkyl-S—(C₁-C₆)alkyl- and —(C₀-C₆)alkyl-NR¹²—(C₁-C₆)alkyl-, wherein R¹² is hydrogen or —(C₁-C₆)alkyl optionally substituted with hydroxy;
M₁ and M₂ are each independently selected from the group of hydrogen, —CN, —OH, —NR¹⁵R¹⁶, —OR¹⁵, and an optionally substituted 6 membered ring selected from the group of aryl and heteroaryl
R⁸, R⁹, R¹², R¹⁵ and R¹⁶ are each independently hydrogen or an optionally substituted radical selected from the group of —(C₁-C₆)alkyl and aryl
aryl is phenyl; and
wherein the optional substitution refers to one or more substituents selected from the group of hydroxy; (C₁-C₆)alkyloxy, aryl, heterocycle, halo, trifluoromethyl, amino, mono- and di-((C₁-C₆)alkylcarbonyl)amino, (C₁-C₆)alkylsulfonyl and aminosulfonyl.

44. A compound according to claim 33 wherein
X is —S—;
Z₁ is N, Z₂ is C, Z₃ is N or C, and Z₄ is C A is selected from the group of hydrogen; halo; —(C₁-C₆)alkyl; —O—(C₁-C₆)alkyl and —(C₀-C₆)alkyl-NR⁸R⁹ wherein R⁸ and R⁹ are each independently hydrogen or —(C₁-C₆)-alkyl;
n is an integer, equal to 1 or 2;
R¹ is —(C₁-C₆)alkyl or a radical V₁-T₁-M₁;
T₁ is a covalent bond;
V₁ is selected from the group of a covalent bond; C(═O) and —(C₁-C₆)alkyl- optionally substituted with hydroxy;
M₁ is selected from the group of hydrogen; —OH; —NR¹⁵R¹⁶ wherein R¹⁵ and R¹⁶ are each independently hydrogen or —(C₁-C₆)alkyl; —OR¹⁵, wherein R¹⁵ is —(C₁-C₆)alkyl; and phenyl;
V₂ is selected from the group of a covalent bond; —(C₀-C₆)alkyl-NR¹²—(C₁-C₆)alkyl-, wherein R¹² is hydrogen or —(C₁-C₆)alkyl optionally substituted with hydroxy; and —(C₀-C₆)alkyl-S—(C₁-C₆)alkyl-; and
M₂ is selected from the group of phenyl; —CN; benzopiperidinyl; pyridinyl; thienyl piperidinyl; furyl; OR¹⁵ wherein R¹⁵ is phenyl or —(C₁-C₆)alkyl —NR¹⁵R¹⁶ wherein R¹⁵ and R¹⁶ are each independently hydrogen or phenyl —C(═O)R¹⁵ wherein R¹⁵ is phenyl and wherein each alkyl- and phenyl-moiety is optionally substituted with one or two radicals selected from the group of methoxy, ethoxy, chloro, fluoro, phenyl, methyl, ethyl, trifluoromethyl, hydroxy, amino, methylcarbonylamino, methylsulfonyl, aminosulfonyl, tetrazolyl, tetrazolyl(C₁-C₆)alkyl and tetrazolyl(C₁-C₆)alkyloxy;
with the proviso that N-(2-(4-methoxyphenyl)ethyl]thieno-(2,3-d)pyrimidin-4-amine and 5-ethyl-6-phenyl-thieno(2,3-d)pyrimidine-2,4-diamine are excluded.

45. A compound according to claim 33, wherein said compound is a pharmaceutically acceptable acid or base addition salt thereof, a stereochemically isomeric form thereof and an N-oxide form thereof with the proviso that N-(2-(4-methoxyphenyl)ethyl)thieno-(2,3-d)pyrimidin-4-amine is excluded.

46. A compound according to claim 33, which exist as optical isomers, wherein said compound is either the racemic mixture or the individual optical isomer.

47. A pharmaceutical composition comprising a therapeutically effective amount of a compound according to claim 33 and a pharmaceutically acceptable carrier and/or excipient.

48. A method for (a) treating or preventing a condition in a mammal affected or facilitated by the neuromodulatory effect of mGluR2 positive allosteric modulators or (b) treating, or preventing, ameliorating, controlling or reducing the risk of various neurological and psychiatric disorders associated with glutamate dysfunction in a mammal affected or facilitated by the neuromodulatory effect of mGluR2 positive allosteric modulators comprising administering to the subject the compound of claim 33.

49. The method of claim 48, wherein the condition or disorder is a central nervous system disorder selected from the group of anxiety disorders, psychotic disorders, personality disorders, substance-related disorders, eating disorders, mood disorders, migraine, epilepsy or convulsive disorders, childhood disorders, cognitive disorders, neurodegeneration, neurotoxicity and ischemia.

50. The method of claim 49, wherein the central nervous system disorder is an anxiety disorder, selected from the group of agoraphobia, generalized anxiety disorder (GAD), obsessive-compulsive disorder (OCD), panic disorder, posttraumatic stress disorder (PTSD), social phobia and other phobias.

51. The method of claim 49, wherein the central nervous system disorder is a psychotic disorder selected from the group of schizophrenia, delusional disorder, schizoaffective disorder, schizophreniform disorder and substance-induced psychotic disorder.

52. The method of claim 49, wherein the central nervous system disorder is a personality disorder selected from the group of obsessive-compulsive personality disorder and schizoid, schizotypal disorder.

53. The method of claim 49, wherein the central nervous system disorder is a substance-related disorder selected from the group of alcohol abuse, alcohol dependence, alcohol withdrawal, alcohol withdrawal delirium, alcohol-induced psychotic disorder, amphetamine dependence, amphetamine withdrawal, cocaine dependence, cocaine withdrawal, nicotine dependence, nicotine withdrawal, opioid dependence and opioid withdrawal.

54. The method of claim 49, wherein the central nervous system disorder is an eating disorder selected from the group of anorexia nervosa and bulimia nervosa.

55. The method of claim 49, wherein the central nervous system disorder is a mood disorder selected from the group of bipolar disorders (I & II), cyclothymic disorder, depression, dysthymic disorder, major depressive disorder and substance-induced mood disorder.

56. The method of claim 49, wherein the central nervous system disorder is migraine.

57. The method of claim 49, wherein the central nervous system disorder is epilepsy or a convulsive disorder selected from the group of generalized nonconvulsive epilepsy, generalized convulsive epilepsy, petit mal status epilepticus, grand mal status epilepticus, partial epilepsy with or without impairment of consciousness, infantile spasms, epilepsy partialis continua, and other forms of epilepsy.

58. The method of claim 49, wherein the childhood disorder is attention-deficit/hyperactivity disorder.

59. The method of claim 49, wherein the central nervous system disorder is a cognitive disorder selected from the group of delirium, substance-induced persisting delirium, dementia, dementia due to HIV disease, dementia due to Huntington's disease, dementia due to Parkinson's disease, dementia of the Alzheimer's type, substance-induced persisting dementia and mild cognitive impairment.

60. The method of claim 49, wherein the central nervous system disorder is selected from the group of anxiety, schizophrenia, migraine, depression, and epilepsy.

61. The method of claim 48, wherein the mGluR2 positive allosteric modulator has an ED₅₀ of about 1 μM or less.

62. The method of claim 48, wherein the compound is used in combination with in combination with an orthosteric agonist of mGluR2.

63. A tracer for imaging a metabotropic glutamate receptor comprising the compound of claim 33.
